(19) United States
(12) Patent Application Publication
(10) Pub. No.: US 2019/0201441 A1
(43) Pub. Date: Jul. 4, 2019

(54) AUTOLOGOUS AND ALLOGENIC MACROPHAGES AND MONOCYTES FOR USE IN THERAPEUTIC METHODS

(71) Applicant: Cellular Approaches, Inc., La Jolla, CA (US)

(72) Inventors: Deborah G. NGUYEN, San Diego, CA (US); Christopher Bruce WILSON, Seattle, WA (US)

(21) Appl. No.: 16/296,677

(22) Filed: Mar. 8, 2019

Related U.S. Application Data
(63) Continuation of application No. 16/115,128, filed on Aug. 28, 2018, now abandoned.
(60) Provisional application No. 62/558,689, filed on Sep. 14, 2017.

(51) Int. Cl. A61K 35/15; A61P 31/04

(52) U.S. Cl. CPC A61K 35/15; A61K 38/217; A61P 31/04

(57) ABSTRACT

Provided herein are innate immune cells for use in therapeutic methods. Also described herein are pharmaceutical compositions comprising innate immune cells for use in the treatment of a variety of diseases including, but not limited to pathogenic infections, pulmonary diseases, inflammatory diseases, autoimmune diseases, and immunodeficiency.

### CROSS-REFERENCE

This application is a continuation of U.S. application Ser. No. 16/115,128 filed on Aug. 28, 2018, which claims priority to U.S. Provisional Application No. 62/558,689, filed Sep. 14, 2017, which application are hereby incorporated by reference in their entireties.

### SUMMARY OF THE INVENTION

Disclosed herein, in certain embodiments, are methods of treating a complicated intra-abdominal infection (cIAI) in an individual in need thereof, comprising: administering to the individual an innate immune cell. In some embodiments, the innate immune cell is allogenic. In some embodiments, the innate immune cell is autologous. In some embodiments, the innate immune cell is a monocyte. In some embodiments, the innate immune cell is a macrophage. In some embodiments, the monocyte is produced by a method comprising isolating monocytes from a population of immune cells extracted from an individual. In some embodiments, the monocyte is produced by a method comprising differentiating a CD34+ hematopoietic stem cell from a peripheral blood sample, a cord blood sample, an apheresis sample, or a bone marrow sample into a monocyte progenitor cell and further differentiating the monocyte progenitor cell into the monocyte. In some embodiments, the monocyte is produced by a method comprising differentiating an embryonic stem cell (ESC) into a monocyte progenitor cell and further differentiating the monocyte progenitor cell into the monocyte. In some embodiments, the monocyte is produced by a method comprising genetically reprogramming a somatic cell into an induced pluripotent stem cell (iPSC) and differentiating the iPSC into the monocyte. In some embodiments, the macrophage is produced by a method comprising isolating macrophages from a tissue or a population of immune cells extracted from an individual. In some embodiments, the macrophage is produced by (a) isolating monocytes from a population of immune cells extracted from an individual; and (b) differentiating the isolated monocytes into macrophages. In some embodiments, the macrophage is produced by differentiating a CD34+ hematopoietic stem cell from a peripheral blood sample, a cord blood sample, an apheresis sample, or a bone marrow sample into a macrophage progenitor cell and further differentiating the macrophage progenitor cell into the macrophage. In some embodiments, the macrophage is produced by differentiating an embryonic stem cell (ESC) into a macrophage progenitor cell and further differentiating the macrophage progenitor cell into the macrophage. In some embodiments, the macrophage is produced by genetically reprogramming a somatic cell into an induced pluripotent stem cell (iPSC) and differentiating the iPSC into the macrophage. In some embodiments, the complicated intra-abdominal infection (cIAI) infection is a bacterial infection. In some embodiments, the complicated intra-abdominal infection (cIAI) infection is a fungal infection. In some embodiments, the bacterial infection comprises intracellular bacteria or extracellular bacteria. In some embodiments, the bacterial infection comprises gram negative bacteria. In some embodiments, the bacterial infection comprises gram positive bacteria. In some embodiments, the bacterial infection comprises aerobic bacteria. In some embodiments, the bacterial infection comprises anaerobic bacteria. In some embodiments, the bacterial infection comprises multi-drug resistant bacteria, extensively drug resistant bacteria, or pan-drug resistant bacteria. In some embodiments, the bacterial infection comprises bacterial that are resistant to an antibacterial selected from the group consisting of: penicillin, ampicillin, carbapenem, fluoroquinolone, cephalosporin, tetracycline, erythromycin, methicillin, gentamicin, vancomycin, imipenem, ceftazidime, levofloxacin, linezolid, daptomycin, ceftaroline, clindamycin, fluconazole, and ciprofloxacin. In some embodiments, the bacterial infection comprises bacteria selected from the group consisting of: Lactobacillus, Klebsiella pneumoniae, Klebsiella pneumoniae resistant to third generation cephalosporin, Klebsiella oxytoca, Klebsiella oxytoca resistant to third generation cephalosporin, Clostridium, Clostridium difficile, Acinetobacter baumannii, Escherichia coli, Escherichia coli resistant to third generation cephalosporin, Pseudomonas, Pseudomonas aeruginosa, Staphylococcus aureus, Streptococcus spp., Streptococcus pyogenes, Enterobacteriaceae, Enterococcus faecium, Enterococcus faecalis, Helicobacter pylori, Streptococcus pneumoniae, Streptococcus agalactiae, Serratia, Stenotrophomonas maltophilia, Corynebacterium, Peptostreptococcus, Peptococcus, Staphylococcus epidermidis, Enterococcus, Enterobacter, Proteus, gram positive anaerobic cocci (GPAC), Bacteroides fragilis, Proteus mirabilis, Bacteroides, Bacteroides resistant to metronidazole, and Morganella morganii. In some embodiments, the bacterial infection comprises Clostridium difficile bacteria. In some embodiments, the bacterial infection comprises Klebsiella pneumoniae bacteria. In some embodiments, the bacterial infection comprises Acinetobacter baumannii bacteria. In some embodiments, the bacterial infection comprises Pseudomonas aeruginosa bacteria. In some embodiments, the bacterial infection comprises methicillin-resistant Staphylococcus aureus (MRSA) bacteria. In some embodiments, the bacterial infection comprises Enterococcus bacteria. In some embodiments, the bacterial infection comprises Enterobacteriaceae bacteria. In some embodiments, the bacterial infection comprises Enterococcus faecalis. In some embodiments, the bacterial infection comprises Escherichia coli. In some embodiments, the fungal infection comprises Candida. In some embodiments, the complicated intra-abdominal infection (cIAI) infection is a hospital-acquired complicated intra-abdominal infection (cIAI). In some embodiments, the population of immune cells is extracted from a peripheral blood sample, a cord blood sample, an apheresis sample, or a bone marrow sample of the individual. In some embodiments, the peripheral blood sample is a mobilized peripheral blood sample or a non-mobilized peripheral blood sample. In some embodiments, differentiating the isolated monocytes into macrophages comprises contacting the isolated monocytes with granulocyte-macrophage (GM-CSF) or macrophage (M-CSF) colony-stimulating factor. In some embodiments, the methods further comprise activating the innate immune cells by contacting the innate immune cells with an activator. In some embodiments, the activator is selected from: a small molecule drug, an endotoxin, a cytokine, a chemokine, an interleukin, a pattern recognition receptor (PRR) ligand, a toll-like receptor (TLR) ligand, an adhesion molecule, or any combinations thereof. In some embodiments, the small molecule drug is phorbol myristate acetate. In some embodiments, the endotoxin is lipopolysaccharide (LPS) or delta endotoxin. In some embodiments, the cytokine is IL-4, IL-13, interferon gamma (IFNγ), or tumor-necrosis factor (TNF). In some embodiments, wherein the adhesion molecule is an integrin, an immunoglobulin, or a selectin. In some embodiments, the innate immune cell is genetically engineered to reduce or inhibit production of an unwanted protein, an unwanted amino acid sequence, an unwanted nucleic acid, or an alloantigen. In some embodiments, the unwanted protein is SIRP-α. In some embodiments, the unwanted amino acid sequence is immunoreceptor tyrosine-based inhibition motif (ITIM). In some embodiments, the innate immune cell is frozen. In some embodiments, the complicated intra-abdominal infection (cIAI) is associated with appendicitis. In some embodiments, the complicated intra-abdominal infection (cIAI) is associated with intra-abdominal sepsis. In some embodiments, the complicated intra-abdominal infection (cIAI) is associated with peritonitis. In some embodiments, the complicated intra-abdominal infection (cIAI) is associated with an intra-abdominal abscess. In some embodiments, the complicated intra-abdominal infection (cIAI) is associated with, abdominal surgery. In some embodiments, the complicated intra-abdominal infection (cIAI) is associated with a gastrointestinal perforation. In some embodiments, the complicated intra-abdominal infection (cIAI) is associated with cholecystitis. In some embodiments, the complicated intra-abdominal infection (cIAI) is associated with diverticulitis. In some embodiments, the complicated intra-abdominal infection (cIAI) is associated with a postoperative abdominal infection. In some embodiments, the complicated intra-abdominal infection (cIAI) is associated with colorectal surgery.

### BRIEF DESCRIPTION OF THE DRAWINGS

The novel features of the subject matter disclosed herein are set forth with particularity in the appended claims. A better understanding of the features and advantages of the subject matter disclosed herein will be obtained by reference to the following detailed description that sets forth illustrative embodiments, in which the principles of the subject matter disclosed herein are utilized, and the accompanying drawings of which:

FIG. 1 illustrates the concept of the therapies described herein.

FIGS. 2A, 2B, and 2C show mouse bone marrow-derived macrophages stimulated with interferon gamma (IFNγ) (squares) with an enhanced ability to kill virulent bacterial strains, as evidenced by a decrease in intracellular bacterial burden (CFU=colony forming units). FIG. 2A shows the enhanced killing with the clinically relevant species Pseudomonas aeruginosa. FIG. 2B shows the enhanced killing with the clinically relevant species Acinetobacter baumannii. FIG. 2C shows the enhanced killing with the clinically relevant multidrug resistant clinical isolate of Acinetobacter baumannii (ACI-3). Data shown in FIGS. 2A-C is an average of 6 technical replicates from each of 4 biological replicates.

FIGS. 3A, 3B, and 3C show human monocyte-derived macrophages increase the killing of multiple bacterial species. FIG. 3A shows the total bacterial burden over time (t=20 hrs.) before and after exposure to human monocyte-derived macrophages stimulated with interferon gamma (IFNγ; squares). As evidenced by a decrease in intracellular bacterial burden (CFU=colony forming units), human monocyte-derived macrophages stimulated with interferon gamma (IFNγ) had an enhanced ability to kill Pseudomonas aeruginosa. FIG. 3B shows the number of bacteria killed by monocyte-derived macrophages over the course of 2 hrs. The monocyte-derived macrophages obtained from different donors (n=14) and stimulated with IFNγ showed enhanced killing across multiple clinically relevant species, with a correlation between activities against different bacterial species (p=0.002) FIG. 3C compares the number of bacteria killed by human monocyte-derived macrophages stimulated with IFNγ and a control (non-stimulated human monocyte-derived macrophages). IFNγ stimulated human monocyte-derived macrophages to kill A. baumannii in a majority of young adult donors (8 of 10 donors).

FIG. 4 shows the infusion of mouse monocyte-derived macrophages decreases organ bacterial load in vivo. Mice injected intraperitoneally with Acinetobacter baumanni were subsequently injected with either Control (unstimulated; n=10 animals) or Activated (IFNγ stimulated; n=9 animals) mouse-derived macrophages. Animals were sacrificed and bacterial load (CFU=colony forming units) was measured. Animals treated with stimulated macrophages showed significantly lower bacterial burden in multiple organs. Data shown represents technical triplicates from each organ.

FIGS. 5A, 5B, and 5C show data validating the monocyte to macrophage differentiation efficiency. FIG. 5A shows microscopy images of monocytes at Day 1 and differentiated cells at Day 7. FIG. 5B shows flow cytometry data quantifying the expression level of CD206 in the differentiated cells. FIG. 5C shows flow cytometry data quantifying the expression level of CD38 in the differentiated cells.

FIG. 6 shows an in vitro microbiological assay to assess the bacterial killing activity of macrophages.

FIGS. 7A and 7B show expression levels of cell surface markers and cytokines and chemokines secreted into the culture medium. FIG. 7A shows the expression of 64 cell surface markers. FIG. 7B shows the analysis of a panel of cytokines and chemokines secreted into the culture medium.

FIGS. 8A, 8B, and 8C show in vitro functional assay data characterizing the activated macrophages. FIG. 8A shows fluorescence microscopy images and quantification of phagocytic activity of the activated macrophages exposed to heat-killed fluorescently labeled bacteria in the presence and absence of cytochalasin D. FIG. 8B shows reactive oxygen species levels produced by activated and non-activated macrophages. FIG. 8C shows proton efflux rates (PER) by unstimulated and stimulated macrophages.

FIGS. 9A and 9B show in vitro data of macrophage bacterial killing activity against bacteria relevant in cIAI. FIG. 9A shows the relative amount of bacteria (in CFUs) killed by fresh activated macrophages after two hours (+2 HR), normalized to time zero (T=0). FIG. 9B shows the relative amount of bacteria (in CFUs) killed by cryopreserved activated macrophages after two hours (+2 HR) normalized to time zero (T=0).

FIGS. 10A, 10B, 10C, and 10D show mouse and human activated macrophage efficacy in vivo in a peritonitis infection rodent model. FIG. 10A shows mice administered with autologous mouse bone-marrow derived macrophages (Autologous Ms BMDM) had a greater survival rate than the negative control (phosphate buffered saline (PBS)). FIG. 10B shows mice administered with allogeneic mouse bone-marrow derived macrophages (Allogeneic Ms BMDM) had a greater survival rate than the negative control (PBS). FIG. 10C shows mice administered with fresh human monocyte-derived macrophages (Fresh Human MDM) had a greater survival rate than the negative control (PBS). FIG. 10D shows mice administered with cryopreserved human monocyte-derived macrophages (Cryopreserved Human MDM) had a greater survival rate than the negative control (PBS).

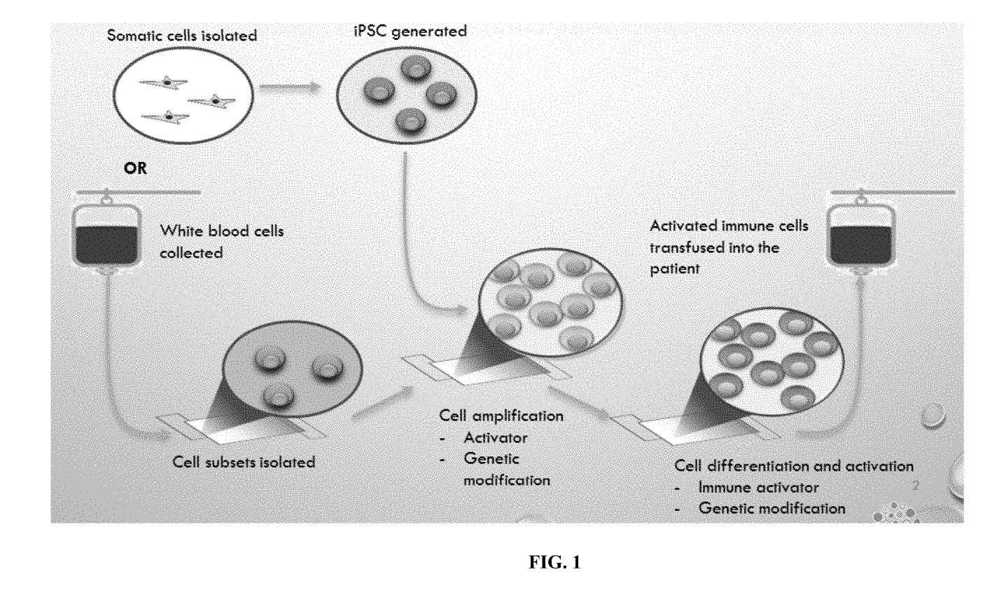

### DETAILED DESCRIPTION OF THE INVENTION

While preferred embodiments of the subject matter disclosed herein have been shown and described herein, it will be obvious to those skilled in the art that such embodiments are provided by way of example only. Numerous variations, changes, and substitutions will now occur to those skilled in the art without departing from the subject matter disclosed herein. It should be understood that various alternatives to the embodiments of the subject matter disclosed herein may be employed in practicing the subject matter disclosed herein. It is intended that the following claims define the scope of the subject matter disclosed herein and that methods and structures within the scope of these claims and their equivalents be covered thereby.

### Definitions

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “subject,” “individual,” “host,” “donor,” and “patient” are used interchangeably herein to refer to a vertebrate, for example, a mammal. Mammals include, but are not limited to, murine, simians, humans, farm animals, sport animals, and pets. Tissues, cells, and their progeny of a biological entity obtained in vivo or cultured in vitro are also encompassed. Designation as a “subject,” “individual,” “host,” “donor,” or “patient” does not necessarily entail supervision of a medical professional.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “therapeutically effective amount” refers to an amount of an immunological cell or a pharmaceutical composition described herein that is sufficient and/or effective in achieving a desired therapeutic effect in treating a patient having a pathogenic disease. In some embodiments, a therapeutically effective amount of the immune cell will avoid adverse side effects.

As used herein, the term “pluripotent stem cells” (PSCs) refers to cells capable, under appropriate conditions, of producing different cell types that are derivatives of all of the 3 germinal layers (i.e. endoderm, mesoderm, and ectoderm). Included in the definition of pluripotent stem cells are embryonic stem cells of various types including human embryonic stem (hES) cells, human embryonic germ (hEG) cells; non-human embryonic stem cells, such as embryonic stem cells from other primates, such as Rhesus stem cells, marmoset stem cells; murine stem cells; stem cells created by nuclear transfer technology, as well as induced pluripotent stem cells (iPSCs).

As used herein, the term “embryonic stem cells” (ESCs) refers to pluripotent stem cells that are derived from a blastocyst before substantial differentiation of the cells into the three germ layers (i.e. endoderm, mesoderm, and ectoderm). ESCs include any commercially available or well-established ESC cell line such as H9, H1, H7, or SA002.

As used herein, the term “induced pluripotent stem cells” or “iPSCs” refers to somatic cells that have been reprogrammed into a pluripotent state resembling that of embryonic stem cells. Included in the definition of iPSCs are iPSCs of various types including human iPSCs and non-human iPSCs, such as iPSCs derived from somatic cells that are primate somatic cells or murine somatic cells.

As used herein, the term “allogenic” means the plurality of macrophages are obtained from a genetically non-identical donor. For example, allogenic macrophages are extracted from a donor and returned back to a different, genetically non-identical recipient.

As used herein, the term “autologous” means the plurality of macrophages are obtained from a genetically identical donor. For example, autologous macrophages are extracted from a patient and returned back to the same, genetically identical patient.

As used herein, the term “activated” and “stimulated” are used interchangeably to indicate that an immune cell (e.g. a macrophage or a monocyte) is exposed to or contacted with an activator.

As used herein, the term “non-activated” and “unstimulated” are used interchangeably to indicate that an immune cell (e.g. a macrophage or a monocyte) is not exposed to or not contacted with an activator.

As used herein, the term “activator” is any molecular entity that drives a change in the genome, transcriptome, proteome, or metabolome of a cell.

### Macrophages and Monocytes

The emergence of pathogen resistance to multiple antimicrobial and antibiotic agents has become a significant public threat that places substantial clinical and financial burden on health care systems and patients. Recent statistic reports show pathogenic infections are the largest addressable hospital cost in the United States. In addition, pathogenic infections and sepsis are the leading cause of death in non-cardiac Intensive Care Units (ICUs). Thus, there is a clear need for alternative methods of management, prevention, and resolution of pathogenic infections and sepsis, including those caused by multi-, extensively, and pan-drug resistant pathogens. Disclosed herein, in certain embodiments, are methods of treating a pathogenic infection in an individual in need thereof comprising the administration of macrophages or monocytes to the individual.

Macrophages and monocytes are part of the innate immune system. The innate immune system is an important component of the overall immune system that provides protection to the host from foreign pathogens. Unlike the adaptive immune system, an innate immune response does not develop over time against a specific pathogenic antigen or epitope the way an adaptive immune response does. However, the innate immune system is quick to recognize and respond within the first few critical hours and days of exposure to a new pathogen. The innate immune system comprises a group of proteins and phagocytic cells, including macrophages and monocytes, which recognize conserved features of pathogens and become activated when these conserved features are encountered.

### Macrophages

Macrophages are a type of white blood cell that engulfs and digests pathogenic organisms. Macrophages recognize foreign pathogens for uptake through several mechanisms, including both non-specific bulk endocytosis and through engagement of specific receptors on the cell surface that either bind to epitopes on the bacterial surface itself or bind mammalian proteins that have bound to the bacterial surface (antibodies, complement proteins, or other opsonins). Following internalization of a pathogen by the macrophage, the pathogen becomes encapsulated in a membrane bound compartment called the phagosome. The phagosome is fused with a lysosome to form a phagolysosome. The phagolysosome contains enzymes, reactive oxygen species, and other toxic molecules that break-down the pathogen. Macrophages also internalize and breakdown infected cells and cell debris from the site of an active infection, helping prevent further spread of the infection and limiting the area of tissue damage.

Macrophages also play a role in innate immunity and adaptive immunity by recruiting other immune cells to the site of an infection. For example, macrophages function as antigen presenting cells to T cells. Following phagocytosis and degradation of a pathogen, a macrophage will present an antigen of the pathogen for helper T cells in the context of the major histocompatibility complex (MHC) class II proteins on the cell surface. Analogously, viral pathogens replicating within macrophages can also be degraded and presented on the MHC class I complex at the cell surface. Presentation of the antigen by the macrophage together with appropriate co-stimulatory proteins results in the activation of T cells and subsequent production of antibodies that target the antigen. Macrophages also recruit and activate other immune cell types by secreting soluble factors like cytokines and chemokines, which signal to other circulating immune cells to infiltrate the infected area and help fight the infection.

Macrophages are either derived from the proliferation of specialized tissue macrophage populations (e.g. Kupffer cells) or differentiate from circulating peripheral-blood monocytes, which migrate into tissue in the steady state or in response to inflammation. Monocytes develop from myeloid progenitor cells in the bone marrow. Myeloid progenitor cells give rise to monoblasts which develop into pro-monocytes which then develop into monocytes. The monocytes are released from the bone marrow into the bloodstream. Once in the bloodstream they migrate into tissues, where they differentiate into macrophages or dendritic cells.

Macrophages are activated via several different pathways. The classical method of activation results in macrophages that are produced during cell-mediated immune responses. Generally, the presence of interferon-γ (IFNγ) and/or tumor-necrosis factor (TNF) in a tissue results in a macrophage population that targets pathogens and secretes high levels of pro-inflammatory cytokines. IFNγ is produced, for example by natural killer (NK) cells in response to stress and infections. The presence of IFNγ activates macrophages to secrete pro-inflammatory cytokines, and to produce increased amounts of superoxide anions and oxygen and nitrogen radicals to increase their killing ability. Macrophages are also classically activated by certain molecular patterns commonly present in pathogenic organisms, such as lipopolysaccharide (LPS) or the nucleic acid CpG. These molecules are recognized by a class of pattern-recognition receptors (PRRs) like the Toll-like receptors (TLRs), leading to an intracellular signaling cascade that ultimately turns on the macrophage pathogen defense response. Macrophages can also be alternatively activated by exposure to cytokines, such as IL-4 and IL-13. Alternatively-activated macrophages produce soluble factors such as IL-10 and matrix metalloproteinases (MMPs) that downregulate pro-inflammatory cytokines like TNF and promote wound healing by breaking down extracellular matrix proteins.

The production of IFNγ by NK cells is transient and results in the transient production of macrophages primed to target pathogens. To assist in the activation of macrophages, adaptive immune cells, such as TH1 cells, are recruited. While T helper 1 (TH1) cells are antigen specific, macrophages activated in response to the TH1 cells can target any pathogenic cells. In some embodiments, the methods disclosed herein further comprise administering NK cells to the individual. In some embodiments, the methods disclosed herein further comprise administering TH1 cells to the individual in need thereof. In some embodiments, the TH1 cells are specific to the unwanted pathogen. In some embodiments, the TH1 cells are not specific to the unwanted pathogen.

In certain instances, pro-inflammatory cytokines produced by classically activated macrophages are associated with damage to the host. IL-1, IL-6, and IL-23 are produced by classically activated macrophages. These cytokines result in the development and expansion of TH17 cells which produce IL-17. Excessive IL-17 levels in tissue are associated with unwanted inflammation and sometimes the progression of an autoimmune phenotype. TNF-alpha and TNFSF1A are additional cytokines produced by classically activated macrophages. Chemokines including IL-8/CXCL8, IP-10/CXCL10, MIP-1 alpha/CCL3, MIP-1 beta/CCL4, and RANTES/CCL5 are produced by classically activated macrophages. In some embodiments, the plurality of macrophages is genetically engineered to reduce or inhibit production of an unwanted cytokine. In some embodiments, the cytokine is selected from TNF, IL-1, IL-6, IL-8, IL-12, and IL-23.

In some embodiments, a macrophage for use in a method disclosed herein is activated before administration by exposure to IL-4, IL-13, interferon-γ (IFNγ), and/or tumor-necrosis factor (TNF) in cell culture, resulting in in vitro activated macrophages. In some embodiments, a macrophage for use in a method disclosed herein is activated before administration by in vitro exposure to IL-4, IL-13, interferon-γ (IFNγ), and/or tumor-necrosis factor (TNF) followed by an additional stimulant, such as bacterial lipopolysaccharide (LPS), resulting in in vitro activated macrophages. In some embodiments, a macrophage for use in a method disclosed herein is activated by exposure to IL-4, IL-13, interferon-γ (IFNγ), and/or tumor-necrosis factor (TNF) in the individual, resulting in in vivo activated macrophages. In some embodiments, a macrophage for use in a method disclosed herein is activated by exposure to IL-4, IL-13, interferon-γ (IFNγ), and/or tumor-necrosis factor (TNF), followed by an additional stimulant, such as a pathogen or pathogen-associated molecular pattern, in the individual, resulting in in vivo activated macrophages. In some embodiments, a macrophage for use in a method disclosed herein is activated by exposure to IL-4, IL-13, interferon-γ (IFNγ), and/or tumor-necrosis factor (TNF), followed by an additional stimulant, such as a TLR agonist, in the individual, resulting in in vivo activated macrophages. In some embodiments, a macrophage for use in a method disclosed herein is activated by exposure to IL-4, IL-13, interferon-γ (IFNγ), and/or tumor-necrosis factor (TNF), followed by an additional stimulant, such as a vaccine adjuvant, in the individual, resulting in in vivo activated macrophages.

### Monocytes

Monocytes are produced in the bone marrow from monoblasts. Monocytes circulate in the bloodstream until they encounter a molecular signal that indicates damage or infection in the nearby tissue. They then migrate out of the blood into the damaged tissue. Chemotaxis of monocytes to a pathogen is controlled by multiple compounds, including monocyte chemotactic protein-1; monocyte chemotactic protein-3 (CCL7); Leukotriene B4; 5-HETE; 5-oxo-ETE); and N-Formylmethionine leucyl-phenylalanine.

Once in a tissue, monocytes can mature into macrophages or dendritic cells. There are several subsets of monocytes in humans as defined by their surface markers, including, classical (CD14⁺⁺CD16⁻), non-classical (CD14^{dim}CD16⁺⁺), and intermediate (CD14⁺⁺CD16⁺). While their downstream functional differences are still unclear, they each have the capacity to differentiate to macrophages under the correct stimulation conditions.

Monocytes themselves engage in phagocytosis and cytokine production. Following opsonization by an opsonin (e.g., an antibody, complement protein, or one of several circulating proteins (e.g., pentraxins, collectins, and ficolins)) monocytes are able to engulf a pathogen. Like macrophages, monocytes are able to phagocytose pathogens by binding directly to pattern-recognition receptors on the pathogen. Monocytes also use antibody-dependent cell-mediated cytotoxicity (ADCC) to kill pathogens.

In some embodiments, a monocyte for use in a method disclosed herein is activated before administration by exposure to IL-4, IL-13, interferon-γ (IFNγ), and/or tumor-necrosis factor (TNF) in cell culture, resulting in in vitro activated monocytes. In some embodiments, a monocyte for use in a method disclosed herein is activated before administration by in vitro exposure to IL-4, IL-13, interferon-γ (IFNγ), and/or tumor-necrosis factor (TNF) followed by an additional stimulant, such as bacterial lipopolysaccharide (LPS), resulting in in vitro activated monocytes. In some embodiments, a monocyte for use in a method disclosed herein is activated by exposure to IL-4, IL-13, interferon-γ (IFNγ), and/or tumor-necrosis factor (TNF) in the individual, resulting in in vivo activated monocytes. In some embodiments, a monocyte for use in a method disclosed herein is activated by exposure to IL-4, IL-13, interferon-γ (IFNγ), and/or tumor-necrosis factor (TNF), followed by an additional stimulant, such as a pathogen or pathogen-associated molecular pattern, in the individual, resulting in in vivo activated monocytes. In some embodiments, a monocyte for use in a method disclosed herein is activated by exposure to IL-4, IL-13, interferon-γ (IFNγ), and/or tumor-necrosis factor (TNF), followed by an additional stimulant, such as a TLR agonist, in the individual, resulting in in vivo activated monocytes. In some embodiments, a monocyte for use in a method disclosed herein is activated by exposure to IL-4, IL-13, interferon-γ (IFNγ), and/or tumor-necrosis factor (TNF), followed by an additional stimulant, such as a vaccine adjuvant, in the individual, resulting in in vivo activated monocytes.

### Isolated and Purified Monocytes and Macrophages

Disclosed herein, in certain embodiments, are isolated and purified innate immune cells. Additionally disclosed herein, in certain embodiments, are pharmaceutical compositions comprising: (a) isolated and purified innate immune cells; and (b) a pharmaceutically-acceptable excipient.

In some embodiments, the innate immune cells are macrophages. In some embodiments, the macrophages are Kupffer cells, histiocytes, alveolar macrophages, splenic macrophages, placental macrophages, peritoneal macrophages, osteoclasts, adipose tissue macrophage (ATM), or sinusoidal lining cells. In some embodiments, the macrophages are produced by a method comprising isolating a subpopulation of macrophages from a population of immune cells extracted from an individual. In some embodiments, the macrophages are produced by a method comprising (a) isolating a subpopulation of macrophage progenitor cells from a population of immune cells extracted from an individual; and (b) differentiating the isolated macrophage progenitor cells into a plurality of macrophages ex vivo. In some embodiments, the macrophages are produced by generating macrophage progenitor cells from embryonic stem cells (ESCs) and differentiating the macrophage progenitor cells into macrophages. In some embodiments, the macrophages are produced by reprogramming somatic cells into induced pluripotent cells (iPSCs), generating macrophage progenitor cells from the iPSCs, and differentiating the macrophage progenitor cells into macrophages.

In some embodiments, the innate immune cells are monocytes. In some embodiments, the monocytes are produced by a method comprising isolating a subpopulation of monocytes from a population of immune cells extracted from an individual. In some embodiments, the monocytes are produced by generating monocyte progenitor cells from embryonic stem cells (ESCs) and differentiating the monocyte progenitor cells into macrophages. In some embodiments, the monocytes are produced by reprogramming somatic cells into induced pluripotent cells (iPSCs), generating monocyte progenitor cells from the iPSCs, and differentiating the monocyte progenitor cells into macrophages.

In some embodiments, the innate immune cells are fresh, i.e., not frozen or previously frozen. In some embodiments, the innate immune cells are frozen and stored for later use (for example to facilitate transport) to generate frozen macrophages or monocytes. In some embodiments, the innate immune cells are administered to the individual after being thawed. In some embodiments, a pharmaceutical formulation disclosed herein comprises (a) isolated and purified innate immune cells; and (b) a cryoprotectant. In some embodiments, a pharmaceutical formulation disclosed herein comprises (a) isolated and purified macrophages; and (b) a cryoprotectant. In some embodiments, a pharmaceutical formulation disclosed herein comprises (a) isolated and purified monocytes; and (b) a cryoprotectant. In some embodiments, the cryoprotectant is selected from dimethylsulfoxide (DMSO), formamide, propylene glycol, ethylene glycol, glycerol, trehalose, 2-methyl-2,4-pentanediol, methanol, butanediol, or any combination thereof. In some embodiments, the cryoprotectant is less than about 100% of the pharmaceutical formulation. In some embodiments, the cryoprotectant is less than about 100% of a buffer. In some embodiments, the cryoprotectant is less than about 100% of a cell culture medium. In some embodiments, the cryoprotectant is less than about 0.5% to about 90% of the pharmaceutical formulation. In some embodiments, the cryoprotectant is less than at least about 0.5% of the pharmaceutical formulation. In some embodiments, the cryoprotectant is less than at most about 90% of the pharmaceutical formulation. In some embodiments, the cryoprotectant is less than about 90% to about 80%, about 90% to about 70%, about 90% to about 60%, about 90% to about 50%, about 90% to about 40%, about 90% to about 30%, about 90% to about 20%, about 90% to about 10%, about 90% to about 5%, about 90% to about 1%, about 90% to about 0.5%, about 80% to about 70%, about 80% to about 60%, about 80% to about 50%, about 80% to about 40%, about 80% to about 30%, about 80% to about 20%, about 80% to about 10%, about 80% to about 5%, about 80% to about 1%, about 80% to about 0.5%, about 70% to about 60%, about 70% to about 50%, about 70% to about 40%, about 70% to about 30%, about 70% to about 20%, about 70% to about 10%, about 70% to about 5%, about 70% to about 1%, about 70% to about 0.5%, about 60% to about 50%, about 60% to about 40%, about 60% to about 30%, about 60% to about 20%, about 60% to about 10%, about 60% to about 5%, about 60% to about 1%, about 60% to about 0.5%, about 50% to about 40%, about 50% to about 30%, about 50% to about 20%, about 50% to about 10%, about 50% to about 5%, about 50% to about 1%, about 50% to about 0.5%, about 40% to about 30%, about 40% to about 20%, about 40% to about 10%, about 40% to about 5%, about 40% to about 1%, about 40% to about 0.5%, about 30% to about 20%, about 30% to about 10%, about 30% to about 5%, about 30% to about 1%, about 30% to about 0.5%, about 20% to about 10%, about 20% to about 5%, about 20% to about 1%, about 20% to about 0.5%, about 10% to about 5%, about 10% to about 1%, about 10% to about 0.5%, about 5% to about 1%, about 5% to about 0.5%, or about 1% to about 0.5% of the pharmaceutical formulation. In some embodiments, the cryoprotectant is less than about 90%, about 80%, about 70%, about 60%, about 50%, about 40%, about 30%, about 20%, about 10%, about 5%, about 1%, or about 0.5% of the pharmaceutical formulation.

In some embodiments, the monocytes are cryopreserved. In some embodiments, the macrophages are cryopreserved. In some embodiments, the macrophages are cryopreserved after being activated. In some embodiments, the activated macrophages are cryopreserved. In some embodiments, the cryopreserved macrophages have a shelf life of about 1 month. In some embodiments, the cryopreserved macrophages have a shelf life of about 2 months. In some embodiments, the cryopreserved macrophages have a shelf life of about 3 months. In some embodiments, the cryopreserved macrophages have a shelf life of about 4 months. In some embodiments, the cryopreserved macrophages have a shelf life of about 5 months. In some embodiments, the cryopreserved macrophages have a shelf life of about 6 months. In some embodiments, the cryopreserved macrophages have a shelf life of about 7 months. In some embodiments, the cryopreserved macrophages have a shelf life of about 8 months. In some embodiments, the cryopreserved macrophages have a shelf life of about 9 months. In some embodiments, the cryopreserved macrophages have a shelf life of about 10 months. In some embodiments, the cryopreserved macrophages have a shelf life of about 11 months. In some embodiments, the cryopreserved macrophages have a shelf life of about 12 months or more.

In some embodiments, the innate immune cells are activated before administration to the individual. In some embodiments, the innate immune cells are not activated before administration to the individual. In some embodiments, the innate immune cells are activated by the immune system of the individual and the presence of a pathogen in the individual. In some embodiments, innate immune cells are co-administered with a compound that activates the innate immune cells in vivo. In some embodiments, a pharmaceutical formulation disclosed herein comprises (a) isolated and purified innate immune cells; and (b) a compound that activates the innate immune cells. In some embodiments, the compound that activates innate immune cells is selected from: IL-4, IL-13, phorbol myristate acetate, lipopolysaccharide (LPS), IFNγ, tumor-necrosis factor (TNF), or any combinations thereof.

In some embodiments, the innate immune cells are autologous to an individual. In some embodiments, the innate immune cells are allogenic. As used herein, “autologous” means the plurality of innate cells are obtained from the individual or a genetically identical donor. As used herein, “allogenic” means the plurality of innate cells are obtained from a genetically non-identical donor.

### Isolation of Monocytes

In some embodiments, monocytes or monocyte progenitor cells are isolated from a human blood sample or a human bone marrow sample. In some embodiments, monocytes or monocyte progenitor cells are isolated from an apheresis sample. In some embodiments, monocyte progenitor cells are differentiated into monocytes in vitro. In some embodiments, the monocyte progenitor cells are hematopoietic stem cells, CD34+ stem cells, common myeloid progenitor cells, or granulocyte-monocyte progenitor cells.

Any suitable means for isolating monocytes or monocyte progenitor cells from an individual is contemplated for use with the methods disclosed herein. Methods to isolate monocytes or monocyte progenitor cells from an individual include, but are not limited to: isolation by adherence, isolation by size sedimentation on Percoll, isolation by flow sorting, positive or negative bead-based selection using cell surface markers, or isolation by counterflow centrifugal elutriation.

In some embodiments, the monocytes or monocyte progenitor cells are isolated from a human blood sample. In some embodiments, the human blood sample is a peripheral blood sample. In some embodiments, the human blood sample is a cord blood sample. In some embodiments, the peripheral blood sample is a mobilized blood sample. In some embodiments, the cord blood sample is a mobilized blood sample. In some embodiments, the peripheral blood sample is a non-mobilized blood sample. In some embodiments, the cord blood sample is a non-mobilized blood sample.

Mobilization is a process where monocytes or monocyte progenitor cells are stimulated out of the bone marrow space into the bloodstream, making them available for collection. Thus, mobilization presents a less invasive alternative to a bone marrow harvest, which is a surgical procedure that is also used as a method to collect macrophage progenitor cells from the bone marrow of the donor. In some embodiments, mobilization is performed by administrating to the donor a drug, a cytokine, a hormone, a protein, or any combination thereof. In some embodiments, mobilization is performed by administrating to the donor granulocyte colony-stimulating factor (G-CSF), granulocyte macrophage colony-stimulating factor (GM-CSF), plerixafor, stem cell factor (SCF), a CXCR4 inhibitor, an SW agonist, a VCAM inhibitor, a VLA-4 inhibitor, a parathyroid hormone, a proteosome inhibitor, growth regulated protein beta (Groβ), a HIF stabilizer, or any combination thereof.

In some embodiments, a blood sample is obtained from an individual. In some embodiments, an apheresis sample is obtained from an individual. In some embodiments, the individual is a healthy individual (i.e., a healthy donor). In some embodiments, the healthy donor is pre-screened and human leukocyte antigen (HLA)-typed as outlined in Code of Federal Regulations (CFR) Title 21, Part 1271, Subpart C (21CFR1271.C). In some embodiments, apheresis or leukapheresis is performed after obtaining the human blood sample. Leukapheresis is a procedure in which white blood cells are separated from a blood sample, allowing the return of red blood cells to the donor. In some embodiments, the blood sample undergoes apheresis and produces an apheresis product. In some embodiments, monocytes are isolated from the apheresis product. In some embodiments, CD14+ cells are isolated from the apheresis product. In some embodiments, CD14+ monocytes are isolated from the apheresis product. In some embodiments, the apheresis product is a plurality of peripheral blood mononuclear cells (PBMCs). In some embodiments, the monocytes, CD14+ cells, and/or the CD14+ monocytes are isolated from the PBMCs using magnetic separation.). In some embodiments, the monocytes, CD14+ cells, and/or the CD14+ monocytes are isolated from the PBMCs using magnetic microbeads.

In some embodiments, a peripheral blood sample is obtained from the individual. In some embodiments, the peripheral blood sample is subjected to gradient centrifugation to generate a buffy coat fraction (i.e., the fraction of an anticoagulated blood sample that contains white blood cells). In some embodiments, the buffy coat fraction is subjected to gradient centrifugation in the presence of Ficoll to generate a peripheral blood mononuclear cell (PBMC) fraction. In some embodiments, the PBMC fraction is suspended in a suitable solution (e.g., PBS-EDTA) and centrifuged to generate an isolated PBMC pellet. In some embodiments, the isolated PBMC pellet is suspended in a suitable solution (e.g., RPMI 1640 medium or X-VIVO) to generate a solution of isolated PBMCs.

In some embodiments, monocytes or monocyte progenitor cells are isolated from the solution of isolated PBMCs. In some embodiments, the monocytes or monocyte progenitor cells are positively selected by using beads coated with antibody against common surface markers. In some embodiments, the monocytes are positively selected by using beads coated with an antibody against CD14. In some embodiments, the beads are magnetic microbeads. In some embodiments, the beads are GMP-grade microbeads. In some embodiments, the beads are magnetic, GMP-grade, CD14 microbeads. Exemplary monocyte markers for use in cell sorting include, but are not limited to CD2, CD31, CD56, CD62L, CD192, CX3CR1, CXCR3, CXCR4, CD14, CD16, CD64, CD11b, CD115, Gr-1, Ly-6C, CD204 or any combination thereof. In some embodiments, the monocytes or monocyte progenitor cells are negatively selected by using beads coated with antibodies against common surface markers of cells other than monocytes or monocyte progenitors. Exemplary non-monocytic markers for use in negative selection include, but are not limited to CD3, CD4, CD8, CD19, CD20, BCR, TCR, IgD, IgM, CD56, or any combination thereof. In some embodiments, the monocytes or monocyte progenitor cells are isolated by use of cell sorting, e.g. fluorescence activated cell sorting (FACS). Exemplary monocyte markers for use in cell sorting include, but are not limited to CD2, CD31, CD56, CD62L, CD192, CX3CR1, CXCR3, CXCR4, CD14, CD16, CD64, CD11b, CD115, Gr-1, Ly-6C, CD204 or any combination thereof. Exemplary hematopoietic stem cell markers include, but are not limited to 2B4/CD244/SLAMF4, ABCG2, C1qR1/CD93, CD34, CD38, CD45, CD48/SLAMF2, CDCP1, CXCR4, Flt-3/Flk-2, SCF R/c-kit, SLAM/CD150, or any combination thereof. Exemplary common myeloid progenitor cell markers include, but are not limited to CD34, Flt-3/Flk-2, SCF R/c-kit, IL-3 Rα, or any combination thereof. Exemplary common granulocyte-macrophage progenitor cell markers include, but are not limited to CD34, CD38, IL-3 Rα, or any combination thereof.

Alternatively, in some embodiments, the solution of isolated PBMCs is subjected to gradient centrifugation in the presence of Percoll solution. In some embodiments, the monocyte or monocyte progenitor cell fraction is isolated, suspended in a suitable solution (e.g., PBS-EDTA) and centrifuged to generate an isolated monocyte pellet or monocyte progenitor cell pellet. The pellet is suspended in a suitable solution (e.g., RPMI 1640 medium or X-VIVO) to generate a solution of isolated monocytes or monocyte progenitor cells.

In some embodiments, a highly pure population of monocytes is isolated from the PBMCs. In some embodiments, the monocyte population isolated from the PBMCs comprises about 94% of CD14+ monocytes. In some embodiments, the monocyte population isolated from the PBMCs comprises about 85% to about 100% of CD14+ monocytes. In some embodiments, the monocyte population isolated from the PBMCs comprises at least about 85% of CD14+ monocytes. In some embodiments, the monocyte population isolated from the PBMCs comprises at most about 100% of CD14+ monocytes. In some embodiments, the monocyte population isolated from the PBMCs comprises about 85% to about 90%, about 85% to about 91%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 91% to about 92%, about 91% to about 93%, about 91% to about 94%, about 91% to about 95%, about 91% to about 96%, about 91% to about 97%, about 91% to about 98%, about 91% to about 99%, about 91% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100% of CD14+ monocytes. In some embodiments, the monocyte population isolated from the PBMCs comprises about 85%, about 90%, about 91%, about 92%, about 93%, about 94%, about 95%, about 96%, about 97%, about 98%, about 99%, or about 100% of CD14+ monocytes.

Differentiation of Macrophages from Monocyte Progenitor Cells

In some embodiments, the isolated monocyte progenitor cells are grown in cell culture to generate isolated monocytes. In some embodiments, the monocyte progenitor cells are differentiated into macrophages. In some embodiments, the monocyte progenitor cells are differentiated first into isolated monocytes and subsequently into macrophages. In some embodiments, the monocyte progenitor cells are differentiated directly into macrophages. In some embodiments, the culture is grown in the presence of culture medium comprising RPMI 1640 medium or X-VIVO. In some embodiments, the culture medium is TexMACs, Macrophages SFM, DMEM, macrophage medium, or any other suitable medium for the culture of macrophages. In some embodiments, the culture medium further comprises serum, for example fetal calf serum (FCS) or fetal bovine serum (FBS). In some embodiments, the culture medium comprises a suitable serum replacement that is safe for clinical use, for example human AB serum, human platelet lysate, or chemically defined optimized serum-free medium. In some embodiments, the culture medium further comprises an antibiotic including: actinomycin D, ampicillin, carbenicillin, cefotaxime, fosmidomycin, gentamicin, kanamycin, neomycin, penicillin streptomycin (Pen Strep), polymixyn B, or streptomycin.

In some embodiments, the isolated monocyte progenitor cells are differentiated into monocytes. In some embodiments, monocyte progenitor cells are differentiated into monocytes by contacting monocyte progenitor cells with IL-3, granulocyte macrophage colony-stimulating factor (GM-CSF), macrophage colony-stimulating factor (M-CSF), granulocyte colony-stimulating factor (G-CSF), stem cell factor (SCF), thrombopoietin (TPO), or any combination thereof. In some embodiments, the monocyte progenitor cells are contacted with any possible combination of factors selected from the group comprising: IL-3, GM-CSF, M-CSF, G-CSF, SCF, and/or TPO. For example, in some embodiments, the monocyte progenitor cells are contacted with a combination of IL-3 and GM-CSF; a combination of IL-3 and M-CSF; or a combination of SCF, TPO, G-CSF, and GM-CSF.

In some embodiments, the monocyte progenitor cells are contacted with M-CSF at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, the monocyte progenitor cells are contacted with M-CSF at a concentration ranging from about 50 ng/ml to about 200 ng/ml; from about 200 ng/ml to about 500 ng/ml; from about 500 ng/ml to about 1000 mg/ml.

In some embodiments, the monocyte progenitor cells are contacted with GM-CSF at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of GM-CSF are used, e.g., from about 50 ng/ml to about 200 ng/ml; from about 200 ng/ml to about 500 ng/ml; from about 500 ng/ml to about 1000 mg/ml.

In some embodiments, the monocyte progenitor cells are contacted with G-CSF at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of G-CSF are used, e.g., from about 50 ng/ml to about 200 ng/ml; from about 200 ng/ml to about 500 ng/ml; from about 500 ng/ml to about 1000 mg/ml.

In some embodiments, the monocyte progenitor cells are contacted with IL-3 at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of IL-3 are used, e.g., from about 50 ng/ml to about 200 ng/ml; from about 200 ng/ml to about 500 ng/ml; from about 500 ng/ml to about 1000 mg/ml.

In some embodiments, the monocyte progenitor cells are contacted with SCF at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, the monocyte progenitor cells are contacted with SCF at a concentration ranging from about 50 ng/ml to about 200 ng/ml; from about 200 ng/ml to about 500 ng/ml; from about 500 ng/ml to about 1000 mg/ml.

In some embodiments, the monocyte progenitor cells are contacted with TPO at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, the monocyte progenitor cells are contacted with TPO at a concentration ranging from about 50 ng/ml to about 200 ng/ml; from about 200 ng/ml to about 500 ng/ml; from about 500 ng/ml to about 1000 mg/ml.

In some embodiments, the monocytes are administered to the individual. In some embodiments, the monocytes are fresh, i.e., not frozen. In some embodiments, the monocytes are frozen and stored for later use (for example to facilitate transport). In some embodiments, cell freezing or cryopreservation media and/or cryoprotective agents are utilized to preserve the monocytes during the freezing process. In some embodiments, the cryoprotectant is selected from dimethylsulfoxide (DMSO), formamide, propylene glycol, ethylene glycol, glycerol, trehalose, 2-methyl-2,4-pentanediol, methanol, butanediol, or any combination thereof. In some embodiments, the frozen monocytes are administered to the individual after being thawed.

Differentiation of Macrophages from Isolated Monocytes

In some embodiments, the isolated monocytes are differentiated into macrophages in culture. In some embodiments, the isolated monocytes are obtained from a population of monocyte progenitor cells. In some embodiments, the monocytes are contacted with granulocyte-macrophage (GM-CSF) or macrophage colony-stimulating factor (M-CSF) to generate differentiated macrophages. In some embodiments, the concentration of M-CSF is from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of M-CSF are used, e.g., from about 50 ng/ml to about 200 ng/ml; from about 200 ng/ml to about 500 ng/ml; from about 500 ng/ml to about 1000 mg/ml.

In some embodiments, the monocytes are contacted with M-CSF for about 12 hours to generated differentiated macrophages. In some embodiments, the monocytes are contacted with M-CSF for about 1 day to generated differentiated macrophages. In some embodiments, the monocytes are contacted with M-CSF for about 2 days to generated differentiated macrophages. In some embodiments, the monocytes are contacted with M-CSF for about 3 days to generated differentiated macrophages. In some embodiments, the monocytes are contacted with M-CSF for about 4 days to generated differentiated macrophages. In some embodiments, the monocytes are contacted with M-CSF for about 5 days to generated differentiated macrophages. In some embodiments, the monocytes are contacted with M-CSF for about 6 days to generated differentiated macrophages. In some embodiments, the monocytes are contacted with M-CSF for about 7 days to generated differentiated macrophages.

In some embodiments, the concentration of GM-CSF is from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of GM-CSF are used, e.g., from about 50 ng/ml to about 200 ng/ml; from about 200 ng/ml to about 500 ng/ml; from about 500 ng/ml to about 1000 mg/ml.

In some embodiments, the monocytes are contacted with GM-CSF for about 12 hours to generate differentiated macrophages. In some embodiments, the monocytes are contacted with GM-CSF for about 1 day to generate differentiated macrophages. In some embodiments, the monocytes are contacted with GM-CSF for about 2 days to generate differentiated macrophages. In some embodiments, the monocytes are contacted with GM-CSF for about 3 days to generate differentiated macrophages. In some embodiments, the monocytes are contacted with GM-CSF for about 4 days to generate differentiated macrophages. In some embodiments, the monocytes are contacted with GM-CSF for about 5 days to generate differentiated macrophages. In some embodiments, the monocytes are contacted with M-CSF for about 6 days to generate differentiated macrophages. In some embodiments, the monocytes are contacted with GM-CSF for about 7 days to generate differentiated macrophages.

In some embodiments, the isolated monocytes are grown in cell culture to generate macrophages. In some embodiments, the isolated monocytes and/or the macrophages are grown in the presence of culture medium comprising RPMI 1640 medium or X-VIVO. In some embodiments, the culture medium is TexMACs, Macrophages SFM, DMEM, macrophage medium, or any other suitable medium for the culture of macrophages. In some embodiments, the culture medium further comprises serum, for example fetal calf serum (FCS) or fetal bovine serum (FBS). In some embodiments, the culture medium comprises a suitable serum replacement that is safe for clinical use, for example human AB serum, human platelet lysate, or chemically defined optimized serum-free medium. In some embodiments, the culture medium further comprises an antibiotic including: actinomycin D, ampicillin, carbenicillin, cefotaxime, fosmidomycin, gentamicin, kanamycin, neomycin, penicillin streptomycin (Pen Strep), polymixyn B, or streptomycin. In some embodiments, the cell culture medium is a GMP-grade cell culture medium. In some embodiments, the cell culture medium that consists of GMP-grade components. Non-limiting examples of the GMP-grade components include GMP-grade proteins, GMP-grade cytokines, GMP-grade antibiotics, GMP-grade serum, GMP-grade chemokines, GMP-grade growth factors, and GMP-grade small molecules.

In some embodiments, the monocytes are grown using a cell culture medium devoid of any xenobiotic additives. In some embodiments, the monocytes are expanded in the presence of a cell culture medium devoid of any xenobiotic additives. In some embodiments, the monocytes are not contacted with any xenobiotic additives during culture. In some embodiments, the monocytes proliferate in the presence of a cell culture medium devoid of any xenobiotic additives. In some embodiments, the monocytes are cultivated under xenobiotic-free culture. In some embodiments, administration of monocytes to an individual does not elicit a xenobiotic immune response in the individual. In some embodiments, the monocytes are cultured using a Current Good Manufacturing Practice (CGMP) manufacturing process. In some embodiments, the monocytes are manufactured in compliance with CGMP regulations. In some embodiments, the monocytes are cultured with a cell culture medium that consists of GMP-grade components.

In some embodiments, the macrophages are grown using a cell culture medium devoid of any xenobiotic additives. In some embodiments, the macrophages are expanded in the presence of a cell culture medium devoid of any xenobiotic additives. In some embodiments, the macrophages are not contacted with any xenobiotic additives during culture. In some embodiments, the macrophages proliferate in the presence of a cell culture medium devoid of any xenobiotic additives. In some embodiments, the macrophages are cultivated under xenobiotic-free culture. In some embodiments, the macrophages are cultivated under xenobiotic-free culture. In some embodiments, administration of macrophages to an individual does not elicit a xenobiotic immune response in the individual. In some embodiments, the macrophages are cultured using a Current Good Manufacturing Practice (CGMP) manufacturing process. In some embodiments, the macrophages are cultured with a cell culture medium that consists of GMP-grade components. In some embodiments, the macrophages administered to an individual are CGMP-grade macrophages. In some embodiments, the macrophages are manufactured in compliance with CGMP regulations. In some embodiments, the macrophages are manufactured in compliance with the Code of Federal Regulations Title 21, Section 210.1 (21CFR210.1).

In some embodiments, xenobiotic additives comprise xenobiotic cells and/or xenobiotic material. In some embodiments, xenobiotic cells comprise non-human cells. In some embodiments, the non-human cells are rodent cells. In some embodiments, the non-human cells are used as a feeder layer. In some embodiments, the xenobiotic material comprises non-human serum, non-human proteins, non-human carbohydrates, non-human lipids, non-human sterols, non-human hormones, non-human cytokines, non-human chemokines, non-human growth factors, or any combination thereof.

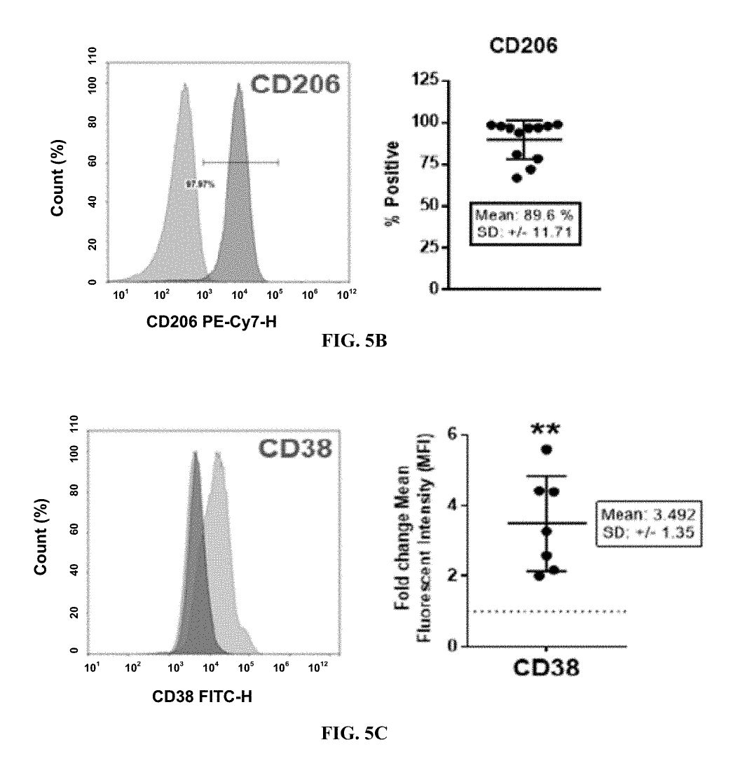

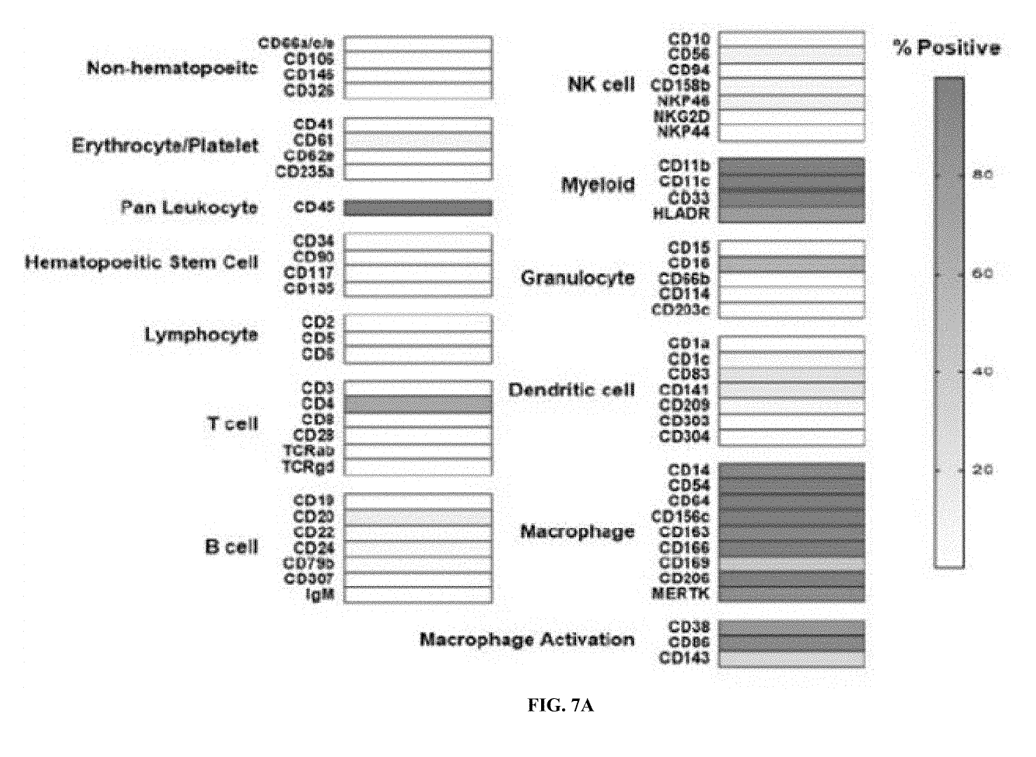

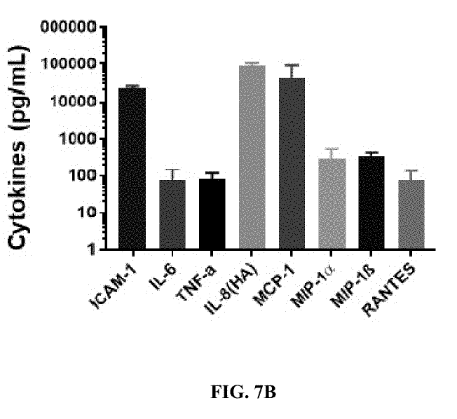

In some embodiments, the differentiated macrophages are isolated by use of cell sorting, e.g. fluorescence activated cell sorting (FACS). Exemplary macrophage markers for use in cell sorting include CD11b, CD68, CD163, F4/80, CD16, CD54, CD49e, CD38, Egr2, CD71, TLR2, TLR4, or any combination thereof. In some embodiments, the differentiated macrophages express macrophage-associated markers, as shown in FIGS. 5B, 5C, and 5A. In some embodiments, the differentiated macrophages express macrophage-associated marker CD206, as shown in FIG. 5B. In some embodiments, the differentiated macrophages express macrophage-associated marker CD163. In some embodiments, the differentiated macrophages express macrophage-associated marker CD63. In some embodiments, the differentiated macrophages express macrophage-associated marker CD14. In some embodiments, the differentiated macrophages express low levels of cell surface markers associated with non-myeloid cell types, as shown in FIG. 7A. In some embodiments, the differentiated macrophages express low levels of non-hematopoietic cell surface markers. In some embodiments, the differentiated macrophages express low levels of erythrocyte and/or platelet cell surface markers. In some embodiments, the differentiated macrophages express low levels of hematopoietic stem cell surface markers. In some embodiments, the differentiated macrophages express low levels of lymphocyte surface markers. In some embodiments, the differentiated macrophages express low levels of B cell surface markers. In some embodiments, the differentiated macrophages express low levels of T cell surface markers. In some embodiments, the differentiated macrophages express low levels of natural killer (NK) cell surface markers. In some embodiments, the differentiated macrophages express low levels of granulocyte cell surface markers. In some embodiments, the differentiated macrophages express low levels of dendritic cell surface markers.

Differentiation of Macrophages from Macrophage Progenitor Cells

In some embodiments, macrophage progenitor cells are differentiated into macrophages in culture. Any suitable means for differentiating the macrophage progenitor cells into macrophages is contemplated for use with the methods disclosed herein. In some embodiments, the macrophage progenitor cells are hematopoietic stem cells, CD34+ hematopoietic stem cells, common myeloid progenitor cells, granulocyte-monocyte progenitor cells, or monocytes. In some embodiments, the macrophage progenitor cells are isolated from an individual. In some embodiments, the macrophage progenitor cells are isolated from a human blood sample, a human tissue, a human peritoneal fluid sample, a human apheresis sample, or a human bone marrow sample. In some embodiments, the macrophage progenitor cells are isolated from a human peripheral blood sample or a human cord blood sample. In some embodiments, the peripheral blood sample is a mobilized blood sample. In some embodiments, the peripheral blood sample is a non-mobilized blood sample. In some embodiments, the cord blood sample is a mobilized blood sample. In some embodiments, the cord blood sample is a non-mobilized blood sample. In some embodiments, the macrophage progenitor cells are isolated from an apheresis sample.

In some embodiments, mobilization is performed by administrating to the donor a drug, a cytokine, a hormone, a protein, or any combination thereof. In some embodiments, mobilization is performed by administrating to the donor granulocyte colony-stimulating factor (G-CSF), granulocyte macrophage colony-stimulating factor (GM-CSF), plerixafor, stem cell factor (SCF), a CXCR4 inhibitor, an SW agonist, a VCAM inhibitor, a VLA-4 inhibitor, a parathyroid hormone, a proteosome inhibitor, growth regulated protein beta (Groβ), a HIF stabilizer, or any combination thereof.

In some embodiments, the macrophage progenitor cells are contacted with a cytokine, a chemokine, a protein, a peptide, a small molecule, a growth factor, or a nucleic acid molecule to generate differentiated macrophages. In some embodiments, the cytokine is macrophage colony-stimulating factor (M-CSF) or stem cell factor (SCF). In some embodiments, the small molecule is FMS-like tyrosine kinase 3 ligand (Flt31). In some embodiments, Flt31 functions as a cytokine and a growth factor. In some embodiments, the protein is GM-CSF, IL-3, or IL-6. In some embodiments GM-CSF functions as a cytokine.

In some embodiments, the nucleic acid molecule is deoxyribonucleic acid (DNA) or ribonucleic acid (RNA). In some embodiments, the RNA molecule is a small RNA. In some embodiments, examples of small RNA include micro-RNA (miRNA), ribosomal RNA (rRNA), small nuclear RNA (snRNA), transfer RNA (tRNA), small nucleolar RNA (snoRNA), Piwi-interacting RNA (piRNA), tRNA-derived small RNA (tsRNA), small rDNA-derived RNA (srRNA), or 5S RNA. In some embodiments, the RNA molecule is a long RNA. In some embodiments, examples of long RNA include long non-coding RNA (lncRNA) or messenger RNA (mRNA). In some embodiments, the RNA molecule is a double stranded RNA (dsRNA), a circular RNA, a small interfering RNA (siRNA), antisense RNA (aRNA), cis-natural antisense transcript (cis-NAT), CRISPR RNA (crRNA), short hairpin RNA (shRNA), trans-acting siRNA (tasiRNA), repeat associated siRNA (rasiRNA), 7SK RNA (7SK), or enhancer RNA (eRNA).

In some embodiments, the macrophage progenitor cells are contacted with granulocyte-macrophage (GM-CSF), macrophage (M-CSF) colony-stimulating factor, FMS-like tyrosine kinase 3 ligand (Flt31), IL-3, IL-6, stem cell factor (SCF), or any combination thereof.

In some embodiments, the macrophage progenitor cells are contacted with M-CSF at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, the macrophage progenitor cells are contacted with M-CSF at a concentration ranging from about 50 ng/ml to about 200 ng/ml; from about 200 ng/ml to about 500 ng/ml; from about 500 ng/ml to about 1000 mg/ml.

In some embodiments, the macrophage progenitor cells are contacted with GM-CSF at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of GM-CSF are used, e.g., from about 50 ng/ml to about 200 ng/ml; from about 200 ng/ml to about 500 ng/ml; from about 500 ng/ml to about 1000 mg/ml.

In some embodiments, the macrophage progenitor cells are contacted with Flt13 at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of Flt13 are used, e.g., from about 50 ng/ml to about 200 ng/ml; from about 200 ng/ml to about 500 ng/ml; from about 500 ng/ml to about 1000 mg/ml.

In some embodiments, the macrophage progenitor cells are contacted with IL-3 at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of IL-3 are used, e.g., from about 50 ng/ml to about 200 ng/ml; from about 200 ng/ml to about 500 ng/ml; from about 500 ng/ml to about 1000 mg/ml.

In some embodiments, the macrophage progenitor cells are contacted with IL-6 at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of IL-6 are used, e.g., from about 50 ng/ml to about 200 ng/ml; from about 200 ng/ml to about 500 ng/ml; from about 500 ng/ml to about 1000 mg/ml.

In some embodiments, the macrophage progenitor cells are contacted with SCF at a concentration ranging from about 1 ng/ml to about 100 ng/ml; e.g. about 5 ng/ml, 10 ng/ml, 25 ng/ml, 50 ng/ml, 75 ng/ml, or 100 ng/ml. In some embodiments, higher concentrations of SCF are used, e.g., from about 50 ng/ml to about 200 ng/ml; from about 200 ng/ml to about 500 ng/ml; from about 500 ng/ml to about 1000 mg/ml.

In some embodiments, the differentiated macrophages are isolated by use of cell sorting, e.g. fluorescence activated cell sorting (FACS). Exemplary macrophage markers for use in cell sorting include CD14, CD11b, CD68, CD163, CD16, CD54, CD49e, CD38, CD204, Egr2, CD71, TLR2, TLR4, or any combination thereof.

Production of Macrophages and Macrophage-Like Cells from Stem Cells

In some embodiments, the macrophages are stem cell-derived macrophages or stem cell-derived macrophage-like cells. In some embodiments, the term “macrophage-like cells” is defined as cells that behave like macrophages, display macrophage markers, function like macrophages, and/or exhibit the same responses as macrophages. In some embodiments, macrophage-like cells express one or more markers selected from CD14, CD11b, CD68, CD163, CD16, CD54, CD49e, CD38, CD204, Egr2, CD71, toll like receptor ligand-2 (TLR2), and TLR4.

In some embodiments, the stem cells are allogenic. In some embodiments, the stem cells are autologous. In some embodiments, the stem cells are embryonic stem (ES) cells. In some embodiments, the embryonic stem cells are H1 ES cells. In some embodiments, the embryonic stem cells are H9 ES cells. In some embodiments, the embryonic stem cells are non-human embryonic stem cells. In some embodiments, stem cells are induced pluripotent stem (iPS) cells. In some embodiments, stem cells are somatic stem cells. In some embodiments, stem cells are pluripotent stem cells. In some embodiments, stem cells are hematopoietic stem cells (HSC). Any suitable means for deriving macrophages or macrophage-like cells from stem cells is contemplated for use with the methods disclosed herein.

### ESC-Derived Macrophages

In some embodiments, embryonic stem (ES) cells are cultured on an irradiated mouse embryonic feeder (MEF) layer in cell culture medium in the presence of leukemia inhibitory factor (LIF). In some embodiments, the cell culture medium is a macrophage differentiation medium (MDM). In some embodiments, the MDM is obtained by culturing fibroblasts and harvesting the medium they are cultured in after reaching confluence. The ES cells form ES cell clusters and in order to induce embryoid body (EB) formation, the ES cell clusters are detached and cultured on a non-adherent cell culture dish without LIF. In some embodiments, ES cells are cultured in a medium supplemented with IL-3. Embryoid bodies (EBs) are generated and they are plated on a gelatin-coated cell culture dish in an adequate cell culture medium. These conditions induce the growth and development of different cell types. After at least 4 days of culture, supernatants of adherent EB contain floating macrophage progenitors. In some embodiments, the macrophage progenitors are collected and plated onto low adherence cell culture dishes. The macrophage progenitors are further cultured for up to 7 days and form an adherent macrophage monolayer. In some embodiments, the macrophage progenitors are cultured in medium, such as RPMI-1640, supplemented with glutamine, fetal bovine serum (FBS), and macrophage differentiation medium. In some embodiments, the ES cell-derived macrophages are harvested from the monolayer by adding a lidocaine solution. In some embodiments, ES cell-derived macrophages express CD11b, CD68, CD163, F4/80, CD16, CD54, CD49e, CD38, Egr2, CD71, TLR-2, TLR-4, or a combination thereof.

iPSC-Derived Macrophages

In some embodiments, a plurality of somatic or adult cells is retrovirally co-transduced with Oct3/4, Sox2, c-Myc, Klf4, and Nanog genes in order to produce induced pluripotent stem (iPS) cells. In some embodiments, retroviral con-transduction with c-Myc or Nanog is not necessary to produce iPS cells. In some embodiments, the somatic or adult cells used to generated iPS cells are human somatic or human adult cells. In some embodiments, the human somatic or human adult cells used to generated iPS cells include fibroblasts, keratinocytes, peripheral blood cells, renal epithelial cells, monocytes, adipose cells, or hepatocytes.

In some embodiments, any cells other than germ cells of mammalian origin (e.g., humans, mice, monkeys, pigs, rats etc.) are used as starting material for the production of iPS cells. Examples include keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), contractile cells (e.g., smooth muscle cells), cells of the blood and the immune system (e.g., T lymphocytes), sense-related cells (e.g., rod cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells thereof (tissue progenitor cells) and the like. There is no limitation on the degree of cell differentiation, the age of the animal from which cells are collected and the like; even undifferentiated progenitor cells (including somatic stem cells) and finally differentiated mature cells can be used alike as sources of somatic cells in the present invention. Examples of undifferentiated progenitor cells include tissue stem cells (somatic stem cells) such as neural stem cells, hematopoietic stem cells, mesenchymal stem cells, and dental pulp stem cells.

Cell colonies displaying iPS cell morphology are cultured passaged on an irradiated mouse embryonic feeder (MEF) layer in an adequate cell culture medium. In some embodiments, the cell colonies displaying iPS cell morphology are cultured in the presence of FGF2. The iPS cells are detached after some days in culture, and in order to induce differentiation, the iPS cells are cultured on a non-adherent cell culture dish, i.e. in feeder-free conditions, without any growth factors. In some embodiments, the human iPS cells are cultured in defined, feeder-free maintenance medium. In some embodiments, the feeder-free maintenance medium is mTeSR™ 1. In some embodiments, the human iPS cells are cultured on Matrigel. In some embodiments, the iPS cells are passaged and plated in medium containing Rho-kinase inhibitor Y-27632.

In some embodiments, embryoid bodies (EBs) are generated by seeding and culturing iPS cells in medium supplemented with BMP-4, stem cell factor, vascular endothelial growth factor (VEGF), and Y-27632. In some embodiments, the EBs are cultured for 4 days. The cells are further expanded in macrophage differentiation media, which induces differentiation of EBs into macrophages. In some embodiments, the macrophage differentiation medium comprises macrophage colony stimulating factor (M-CSF), X-VIVO™ 15, IL-3, glutamax, penicillin, streptomycin, and β-mercaptoethanol. In some embodiments, iPS cell-derived macrophages express wild type macrophage gene markers. In some embodiments, iPS cell-derived macrophages express CD14, CD11b, CD68, CD163, CD16, CD54, CD49e, CD38, CD204, Egr2, CD71, TLR2, TLR4, or combinations thereof.

Production of Monocytes and Monocyte-Like Cells from Stem Cells

In some embodiments, the monocytes administered to the individual are stem cell-derived monocytes or stem cell-derived monocyte-like cells. In some embodiments, the term “monocyte-like cells” is defined as cells that behave like monocytes, display monocyte markers, function like monocytes, and/or exhibit the same responses as monocytes. In some embodiments, monocyte-like cells express one or more markers selected from CD14, CD16, CD36, CD163, Fc receptors CD32 and CD64, CD15, CD33, CD115, CD116, CCR5, CX3CR1, CD34, CCR2.

In some embodiments, the stem cells are allogenic. In some embodiments, the stem cells are autologous. In some embodiments, stem cells are embryonic stem (ES) cells. In some embodiments, the embryonic stem cells are H1 ES cells. In some embodiments, the embryonic stem cells are H9 ES cells. In some embodiments, the embryonic stem cells are non-human embryonic stem cells. In some embodiments, stem cells are induced pluripotent stem (iPS) cells. In some embodiments, stem cells are somatic stem cells. In some embodiments, stem cells are pluripotent stem cells. Any suitable means for deriving monocytes or monocyte-like cells from stem cells is contemplated for use with the methods disclosed herein.

### ESC-Derived Monocytes

In some embodiments, a plurality of embryonic stem (ES) cells is cultured on an irradiated mouse embryonic feeder (MEF) layer in an adequate cell culture medium. In some embodiments, the embryonic stem cells are human embryonic stem cells. In some embodiments, the human embryonic stem cells are H1 (NIH code WA01) or H9 (NIH code WA09). In some embodiments, the adequate cell culture medium is supplemented with fetal bovine serum. In some embodiments, the ES cells form ES cell clusters and in order to induce embryoid body (EB) formation, the ES cell clusters are detached and cultured on a non-adherent cell culture dish. In some embodiments, embryoid bodies (EBs) are generated and they are plated on a gelatin-coated cell culture dish in an adequate cell culture medium. These conditions induce the growth and development of different cell types. After at least 5 days of culture, supernatants of adherent EB contain floating hematopoietic cells. In some embodiments, the EBs are differentiated into a mixture of hematopoietic cells by exposure to an adequate cell culture medium supplemented with bone morphogenetic protein 4 (BMP-4), vascular endothelial growth factor (VEGF), interleukin-3 (IL-3), fetal liver tyrosine kinase 3 ligand (FLT3-L), stem cell factor (SCF), and thrombopoietin. In some embodiments, CD14⁺ cells are isolated from the mixture of hematopoietic cells. In some embodiments, CD14⁺ cells achieve terminal differentiation into a monocyte lineage upon exposure to monocyte-colony-stimulating factor (M-CSF), granulocyte-macrophage-colony-stimulating factor (GM-CSF), IL-3, and FLT3-L. In some embodiments, the ES cell-derived monocytes are collected and further expanded in vitro. In some embodiments, the ES cell-derived monocytes are harvested from the monolayer by adding a lidocaine solution. In some embodiments, hES cell-derived monocytes express wild type monocytic gene markers. In some embodiments, hES cell-derived monocytes express CD14, CD16, CD36, CD163, Fc receptors CD32 and CD64, CD15, CD33, CD115, CD116, CCR5, CX3CR1, CD34, CCR2, or combinations thereof.

iPSC-Derived Monocytes

In some embodiments, a plurality of somatic or adult cells is retrovirally co-transduced with Oct3/4, Sox2, c-Myc, Klf4, and Nanog genes in order to produce induced pluripotent stem (iPS) cells. In some embodiments, retroviral co-transduction with c-Myc or Nanog is not necessary to produce iPS cells. In some embodiments, the somatic or adult cells used to generated iPS cells are human somatic or human adult cells. In some embodiments, the human somatic or human adult cells used to generated iPS cells include fibroblasts, keratinocytes, peripheral blood cells, renal epithelial cells, monocytes, adipose cells, and/or hepatocytes.

In some embodiments, any cells other than germ cells of mammalian origin (e.g., humans, mice, monkeys, pigs, rats etc.) are used as starting material for the production of iPS cells. Examples include keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), contractile cells (e.g., smooth muscle cells), cells of the blood and the immune system (e.g., T lymphocytes), sense-related cells (e.g., rod cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells thereof (tissue progenitor cells) and the like. There is no limitation on the degree of cell differentiation, the age of the animal from which cells are collected and the like; even undifferentiated progenitor cells (including somatic stem cells) and finally differentiated mature cells can be used alike as sources of somatic cells in the present invention. Examples of undifferentiated progenitor cells include tissue stem cells (somatic stem cells) such as neural stem cells, hematopoietic stem cells, mesenchymal stem cells, and dental pulp stem cells.

Cell colonies displaying iPS cell morphology are cultured passaged on an irradiated mouse embryonic feeder (MEF) layer in an adequate cell culture medium. In some embodiments, the cell colonies displaying iPS cell morphology are cultured in the presence of FGF2. The iPS cells are detached after some days in culture, and in order to induce differentiation, the iPS cells are cultured on a non-adherent cell culture dish, i.e. in feeder-free conditions, without any growth factors. In some embodiments, the human iPS cells are cultured in defined, feeder-free maintenance medium. In some embodiments, the feeder-free maintenance medium is mTeSR™ 1. In some embodiments, the human iPS cells are cultured on Matrigel. In some embodiments, the iPS cells are passaged and plated in medium containing Rho-kinase inhibitor Y-27632.

In some embodiments, embryoid bodies (EBs) are generated by seeding and culturing iPS cells in medium supplemented with BMP-4, stem cell factor, vascular endothelial growth factor (VEGF), and Y-27632. In some embodiments, the EBs are cultured for 4 days. The cells are further expanded in a monocyte differentiation medium, which induces differentiation of EBs into monocytes. In some embodiments, the monocyte differentiation medium (MDM) comprises a medium specifically developed to support differentiation of iPS cells such as STEMdiff™ APEL™ medium supplemented with an antibiotic, monocyte (M-CSF) colony-stimulating factor, granulocyte-macrophage colony-stimulating factor (GM-CSF), and IL-3. In some embodiments, non-limiting examples of an antibiotic in the MDM are penicillin, streptomycin sulfate, gentamicin sulfate, neomycin sulfate, polymixin B sulfate, or combinations thereof. Immature myeloid cells are first generated from the EBs exposed to the MDM. Upon longer exposure to the MDM, immature myeloid cells differentiate into monocytes. In some embodiments, hES cell-derived monocytes express wild type monocytic gene markers. In some embodiments, hES cell-derived monocytes express CD14, CD16, CD36, CD163, Fc receptors CD32 and CD64, CD15, CD33, CD115, CD116, CCR5, CX3CR1, CD34, CCR2, or combinations thereof.

### Macrophage and Monocyte Activators

In some embodiments, the innate immune cells described herein are activated.

In some embodiments, a macrophage is activated via exposure to an activator. In some embodiments, a monocyte is activated via exposure to an activator. Any suitable activator is used. In some embodiments, any suitable method of screening a library of compounds is used to identify a macrophage or monocyte activator. In some embodiments, the macrophage or monocyte are activated via in vitro exposure to the activator. In some embodiments, the macrophage or monocyte is activated with the activator in vitro prior to administration to the individual. In some embodiments, exposure of the macrophage or the monocyte to the activator promotes production of a reactive oxygen species, a reactive nitrogen species, or a combination thereof.

In some embodiments, the activator is a small molecule drug, an endotoxin, a cytokine, a chemokine, an interleukin, a pattern recognition receptor (PRR) ligand, a toll-like receptor (TLR) ligand, an adhesion molecule, or any combinations thereof. In some embodiments, the small molecule drug is phorbol myristate acetate. In some embodiments, the cytokine is IL-4, IL-13, interferon gamma (IFNγ), or tumor-necrosis factor (TNF). In some embodiments, the endotoxin is lipopolysaccharide (LPS) or endotoxin delta. In some embodiments, the adhesion molecule is an integrin, an immunoglobulin, or a selectin.

In some embodiments, the activator is a toll-like receptor (TLR) ligand, or a molecule that activates downstream TLR signaling. In some embodiments, the TLR ligand is a ligand that binds to TLR-1, TLR-2, TLR-3, TLR-4, TLR-5, TLR-6, TLR-7, TLR-8, TLR-9, TLR-10, TLR-11, TLR-12, or TLR-13. In some embodiments, the TLR ligand is a ligand that binds to TLR-3 or TLR-4. In some embodiments, the ligand of TLR-3 or TLR-4 is a pathogen-associated molecular pattern (PAMP). In some embodiments, the ligand that binds to TLR-3 is a double-stranded RNA. In some embodiments, the ligand that binds to TLR-4 is a lipopolysaccharide (LPS).

In some embodiments, a macrophage is activated by contacting the macrophage with interferon gamma (IFNγ). In some embodiments, a differentiated macrophage is activated by contacting the macrophage with interferon gamma (IFNγ). In some embodiments, a macrophage is activated by contacting the macrophage with IFNγ for about 6 hours. In some embodiments, a macrophage is activated by contacting the macrophage with IFNγ for about 12 hours. In some embodiments, a macrophage is activated by contacting the macrophage with IFNγ for about 18 hours. In some embodiments, a macrophage is activated by contacting the macrophage with IFNγ for about 24 hours.

In some embodiments, a macrophage is activated by contacting the macrophage with IFNγ for about 1 hour to about 36 hours. In some embodiments, a macrophage is activated by contacting the macrophage with IFNγ for at least about 1 hour. In some embodiments, a macrophage is activated by contacting the macrophage with IFNγ for at most about 36 hours. In some embodiments, a macrophage is activated by contacting the macrophage with IFNγ for about 1 hour to about 3 hours, about 1 hour to about 6 hours, about 1 hour to about 9 hours, about 1 hour to about 12 hours, about 1 hour to about 15 hours, about 1 hour to about 18 hours, about 1 hour to about 21 hours, about 1 hour to about 24 hours, about 1 hour to about 36 hours, about 3 hours to about 6 hours, about 3 hours to about 9 hours, about 3 hours to about 12 hours, about 3 hours to about 15 hours, about 3 hours to about 18 hours, about 3 hours to about 21 hours, about 3 hours to about 24 hours, about 3 hours to about 36 hours, about 6 hours to about 9 hours, about 6 hours to about 12 hours, about 6 hours to about 15 hours, about 6 hours to about 18 hours, about 6 hours to about 21 hours, about 6 hours to about 24 hours, about 6 hours to about 36 hours, about 9 hours to about 12 hours, about 9 hours to about 15 hours, about 9 hours to about 18 hours, about 9 hours to about 21 hours, about 9 hours to about 24 hours, about 9 hours to about 36 hours, about 12 hours to about 15 hours, about 12 hours to about 18 hours, about 12 hours to about 21 hours, about 12 hours to about 24 hours, about 12 hours to about 36 hours, about 15 hours to about 18 hours, about 15 hours to about 21 hours, about 15 hours to about 24 hours, about 15 hours to about 36 hours, about 18 hours to about 21 hours, about 18 hours to about 24 hours, about 18 hours to about 36 hours, about 21 hours to about 24 hours, about 21 hours to about 36 hours, or about 24 hours to about 36 hours. In some embodiments, a macrophage is activated by contacting the macrophage with IFNγ for about 1 hour, about 3 hours, about 6 hours, about 9 hours, about 12 hours, about 15 hours, about 18 hours, about 21 hours, about 24 hours, or about 36 hours.

In some embodiments, the activated macrophage produces a cytokine, as shown in FIG. 7B. In some embodiments, the production of a cytokine by the activated macrophage serves as a measure of anti-bacterial efficacy of the activated macrophage.

In some embodiments, the activated macrophage produces interferon gamma-induced protein 10 (IP-10). In some embodiments, the activated macrophage produces chemokine (C—C motif) ligand 5 (CCL5)/regulated on activation, normal T cell expressed and secreted (RANTES). In some embodiments, the activated macrophage produces tumor necrosis factor alpha (TNFα). In some embodiments, the activated macrophage produces intra-cellular adhesion molecule-1 (ICAM-1). In some embodiments, the activated macrophage produces interleukin-6 (IL-6). In some embodiments, the activated macrophage produces interleukin-8 (IL-8). In some embodiments, the activated macrophage produces monocyte chemoattractant protein-1 (MCP-1). In some embodiments, the activated macrophage produces macrophage inflammatory protein-1 alpha (MIP-1α). In some embodiments, the activated macrophage produces macrophage inflammatory protein-1 beta (MIP-1β).

In some embodiments, the activated macrophage produces about 100 picograms per milliliter (pg/mL) of a cytokine. In some embodiments, the activated macrophage produces about 1,000 pg/mL of a cytokine. In some embodiments, the activated macrophage produces about 10,000 pg/mL of a cytokine. In some embodiments, the activated macrophage produces about 100,000 pg/mL of a cytokine. In some embodiments, the activated macrophage produces about 50 pg/mL to about 1,000,000 pg/mL of a cytokine. In some embodiments, the activated macrophage produces at least about 50 pg/mL of a cytokine. In some embodiments, the activated macrophage produces at most about 1,000,000 pg/mL of a cytokine. In some embodiments, the activated macrophage produces about 50 pg/mL to about 100 pg/mL, about 50 pg/mL to about 500 pg/mL, about 50 pg/mL to about 1,000 pg/mL, about 50 pg/mL to about 5,000 pg/mL, about 50 pg/mL to about 10,000 pg/mL, about 50 pg/mL to about 50,000 pg/mL, about 50 pg/mL to about 100,000 pg/mL, about 50 pg/mL to about 500,000 pg/mL, about 50 pg/mL to about 1,000,000 pg/mL, about 100 pg/mL to about 500 pg/mL, about 100 pg/mL to about 1,000 pg/mL, about 100 pg/mL to about 5,000 pg/mL, about 100 pg/mL to about 10,000 pg/mL, about 100 pg/mL to about 50,000 pg/mL, about 100 pg/mL to about 100,000 pg/mL, about 100 pg/mL to about 500,000 pg/mL, about 100 pg/mL to about 1,000,000 pg/mL, about 500 pg/mL to about 1,000 pg/mL, about 500 pg/mL to about 5,000 pg/mL, about 500 pg/mL to about 10,000 pg/mL, about 500 pg/mL to about 50,000 pg/mL, about 500 pg/mL to about 100,000 pg/mL, about 500 pg/mL to about 500,000 pg/mL, about 500 pg/mL to about 1,000,000 pg/mL, about 1,000 pg/mL to about 5,000 pg/mL, about 1,000 pg/mL to about 10,000 pg/mL, about 1,000 pg/mL to about 50,000 pg/mL, about 1,000 pg/mL to about 100,000 pg/mL, about 1,000 pg/mL to about 500,000 pg/mL, about 1,000 pg/mL to about 1,000,000 pg/mL, about 5,000 pg/mL to about 10,000 pg/mL, about 5,000 pg/mL to about 50,000 pg/mL, about 5,000 pg/mL to about 100,000 pg/mL, about 5,000 pg/mL to about 500,000 pg/mL, about 5,000 pg/mL to about 1,000,000 pg/mL, about 10,000 pg/mL to about 50,000 pg/mL, about 10,000 pg/mL to about 100,000 pg/mL, about 10,000 pg/mL to about 500,000 pg/mL, about 10,000 pg/mL to about 1,000,000 pg/mL, about 50,000 pg/mL to about 100,000 pg/mL, about 50,000 pg/mL to about 500,000 pg/mL, about 50,000 pg/mL to about 1,000,000 pg/mL, about 100,000 pg/mL to about 500,000 pg/mL, about 100,000 pg/mL to about 1,000,000 pg/mL, or about 500,000 pg/mL to about 1,000,000 pg/mL of a cytokine. In some embodiments, the activated macrophage produces about 50 pg/mL, about 100 pg/mL, about 500 pg/mL, about 1,000 pg/mL, about 5,000 pg/mL, about 10,000 pg/mL, about 50,000 pg/mL, about 100,000 pg/mL, about 500,000 pg/mL, or about 1,000,000 pg/mL of a cytokine. Non-limiting examples of the cytokine produced by the activated macrophage include IP-10, CCL5/RANTES, TNFα, ICAM-1, IL-6, IL-8, MCP-1, and MIP-1α, MIP-1β.

In some embodiments, the activated macrophages express markers associated with classical activation. In some embodiments, the activated macrophages express markers associated with alternative activation. In some embodiments, the activated macrophages express markers associated with IFNγ-mediated activation, as shown in FIGS. 5C and 7A. In some embodiments, the activated macrophages express CD38. In some embodiments, the activated macrophages express CD86. In some embodiments, the activated macrophages express CD143. In some embodiments, the activated macrophages express markers associated with LPS-mediated activation. In some embodiments, the activated macrophages express markers associated with cytokine-mediated activation. In some embodiments, the activated macrophages express markers associated with phorbol myristate acetate (PMA)-mediated activation.

### Modification of Innate Immune Cells

In some embodiments, the innate immune cells disclosed herein are modified to reduce or inhibit production of an unwanted protein, an alloantigen, an unwanted nucleic acid sequence, or an unwanted amino acid sequence.

In some embodiments, the protein is signal regulatory protein alpha (SIRPα). In some embodiments, the protein contains an immunoreceptor tyrosine-based inhibition motif (ITIM). SIRPα is a membrane glycoprotein expressed mainly by myeloid cells. SIRPα recognizes and binds to CD47, which triggers intracellular signals through SIRPα's cytoplasmic domain. The cytoplasmic region of SIRPα contains four immunoreceptor tyrosine-based inhibition motifs (ITIMs) that become phosphorylated upon binding. The binding of SIRPα to CD47 results in inhibition of phagocytosis. Therefore, inhibition of SIRPα-CD47 binding in isolated innate immune cells, such as macrophages and monocytes, provides increased phagocytic capabilities of transplanted immune cells. In some embodiments, reduction or inhibition of SIRPα or ITIMs increases phagocytosis of an unwanted pathogen.

In some embodiments, the innate immune cells described herein, such as macrophages and monocytes, are modified to reduce expression of an alloantigen. The term “alloantigens” refers to antigens that differ between members of the same species, when the donor and recipient have different types of major histocompatibility complex (MHC) molecules. In some embodiments, the alloantigens are MHC antigens, blood group antigen, or minor histocompatibility antigens.

In some embodiments, the plurality of innate immune cells, such as macrophages or monocytes, is genetically engineered to express a bacterial, fungal, or viral antigen. In some embodiments, the plurality of innate immune cells is genetically engineered to overexpress relevant receptors that bind to an opsonin. Any suitable method of genetic engineering may be used to produce the plurality of innate immune cells.

### Nucleic Acid Vectors

In some embodiments, the unwanted nucleic acid sequence is a nucleic acid molecule that partially, substantially, or completely deletes, silences, inactivates, or down-regulates a gene encoding an unwanted protein or amino acid sequence (e.g., SIRPα or ITIM) or alloantigen. In some embodiments, the unwanted nucleic acid sequence is introduced into an isolated macrophage or monocyte via an expression vector, under the appropriate conditions, to induce or cause partial, substantial, or complete deletion, silencing, inactivation, or down-regulation of the gene encoding an unwanted protein or amino acid sequence (e.g., SIRPα or ITIM) or alloantigen.

In some embodiments, the unwanted nucleic acid sequence introduced into an isolated macrophage or monocyte via an expression vector, under the appropriate conditions, encodes a bacterial, viral, or fungal antigen. In some embodiments, the bacterial antigen originates from extracellular bacteria. In some embodiments, the bacterial antigen originates from intracellular bacteria. In some embodiments, a bacterial antigen is selected from the bacterial genera comprising: Actinomyces, Bacillus, Bartonella, Bordetella, Borrelia, Bruiella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

In some embodiments, a viral antigen is selected from the group comprising: human immunodeficiency virus (HIV), influenza, hepatitis, varicella, varicella zoster, West Nile, parvovirus, and human papilloma virus. In some embodiments, a fungal antigen is selected from the group comprising: Pneumocystis jirovecii, Candida, Aspergillus, Blastomyces, Cryptococcus gattii, Cryptococcus neformans, Histoplasma, and Coccidioides.

In some embodiments, the components or elements of a vector are optimized such that expression vectors are compatible with the macrophage or the monocyte.

In some embodiments, the macrophage or the monocyte is transformed with a nucleic acid, preferably an expression vector, containing a nucleic acid encoding transcription activator-like effector nucleases (TALEN). TALEN are restriction enzymes are designed to specifically cleave nucleic acid sequences encoding the unwanted protein or amino acid sequence (e.g., SIRPα or ITIM) or alloantigen.

TALEN are produced by the fusion of a transcription activator-like (TAL) effector DNA-binding domain, which is derived from TALE proteins, to a nuclease or Fok1 DNA cleavage domain. Fok1 is a type IIS restriction endonuclease that is naturally found in the gram-negative bacteria Flavobacterium okeanokoites. TALE proteins originate from the bacteria genus Xanthomonas and contain DNA-binding domains, 33-35-amino-acid repeat regions, which are able to recognize a single base pair. This amino acid repeat region in the TAL effectors is readily customizable and determines binding specificity. TALEN bind adjacent DNA target sites and induce double-strand breaks between the target sequences.

In some embodiments, a macrophage or a monocyte is transfected with a vector comprising a nucleic acid sequence encoding TALEN, wherein the TALEN specifically cleaves a nucleic acid sequence encoding an unwanted protein or amino acid sequence (e.g., SIRPα or ITIM) or alloantigen and partially, substantially, or completely deletes, silences, inactivates, or down-regulates the unwanted protein, amino acid sequence, or alloantigen.

In some embodiments, the macrophage or a monocyte is transformed with a nucleic acid, preferably an expression vector, containing a nucleic acid encoding zinc finger nucleases (ZFN). In some embodiments, ZFN are restriction enzymes that can be designed to specifically cleave unwanted nucleic acid sequences encoding an unwanted protein or amino acid sequence (e.g., SIRPα or ITIM) or alloantigen.

ZFN are produced by the fusion of a Cys₂-His₂ zinc finger DNA-binding domain to a DNA-cleavage domain. The DNA-cleavage domain is a Fok1 type IIS restriction endonuclease. The Cys₂-His₂ zinc finger DNA-binding domain is one of the most common DNA-binding motifs found in eukaryotes. An individual zinc finger comprises 30 amino acids and is able to contact three base pairs in the major groove of DNA. Zinc finger DNA-binding domains contain between 3 and 6 zinc finger repeats and can be customized to recognize 9 to 18 target base pairs. In some embodiments, zinc finger DNA-binding domains are generated via a modular assembly process, wherein 3 individual zinc fingers are used to generate a 3-finger array that can recognize 9 target base pairs. In some embodiments, zinc finger DNA-binding domains are generated via a modular assembly process, wherein 2-finger modules are used. In some embodiments, zinc finger DNA-binding domains are generated via a modular assembly process, wherein 1-finger modules are used. ZFN dimers bind adjacent DNA target sites and induce double-strand breaks between the target sequences.

In some embodiments, a macrophage or a monocyte is transfected with a vector containing a nucleic acid sequence encoding a ZFN, wherein the ZFN specifically cleaves a nucleic acid sequence encoding an unwanted protein or amino acid sequence (e.g., SIRPα or ITIM) or alloantigen and partially, substantially, or completely deletes, silences, inactivates, or down-regulates the unwanted protein or amino acid sequence (e.g., TNF, IL-1, IL-6, IL-8, IL-12, and IL-23) or alloantigen.

In some embodiments, a macrophage or a monocyte is transformed with a nucleic acid, preferably an expression vector, encoding a nucleic acid encoding a crRNA, tracrRNA, and a Cas9 molecule. In some embodiments, a macrophage or a monocyte is transformed with a nucleic acid, preferably an expression vector, encoding a Cas9 molecule and a nucleic acid encoding a crRNA and tracrRNA.

The CRISPR/Cas system is originally an RNA-mediated bacterial immune system that provides a form of acquired immunity against viruses and plasmids; it comprises three components: a Cas9 (CRISPR associated protein 9) endonuclease, a crRNA (CRISPR RNA), and a tracrRNA (transactivating crRNA). Clustered regularly interspaced short palindromic repeats (CRISPR) are short repetitions of bacterial DNA followed by short repetitions of spacer DNA from viruses or plasmids. The Cas9 endonuclease contains two nuclease domains and is programmed by a crRNA and tracrRNA hybrid to cleave the target sequence.

In some embodiments, the crRNA sequence is substantially homologous to a portion of the nucleic acid sequence encoding an unwanted protein or amino acid sequence (e.g., SIRPα or ITIM) or alloantigen. In some embodiments the gRNA sequence is substantially homologous to a portion of the nucleic acid sequence encoding an unwanted protein or amino acid sequence (e.g., SIRPα or ITIM) or alloantigen. In some embodiments, the Cas9 endonuclease is programmed by a crRNA and tracrRNA hybrid to cleave the nucleic acid sequence encoding the unwanted protein or amino acid sequence (e.g., SIRPα or ITIM) or alloantigen.

In some embodiments, a nucleic acid molecule that partially, substantially, or completely enhances, activates, or up-regulates a gene encoding a receptor that binds to an opsonin is introduced into an isolated macrophage or monocyte via an expression vector, under the appropriate conditions, to induce or cause partial, substantial, or complete enhancement, activation, or up-regulation of the gene encoding a receptor that binds to an opsonin. In some embodiments, the gene encodes an Fc receptor or a complement receptor 1. In some embodiments, the gene encodes a receptor that binds to an Fc region of an antibody, C3b, C4b, C1q, pentraxin, collectin, ficolin, or combinations thereof.

In some embodiments, the plurality of macrophages or monocytes is transfected with a nucleic acid molecule that partially, substantially, or completely enhances, activates, or up-regulates a gene encoding a receptor that binds to an opsonin. Any of a variety of transfection methods, including non-viral and viral transfection methods, known to the skilled artisan is applicable in the macrophage modification methods. For example, non-viral transfection methods available are chemical-based transfection, non-chemical-based transfection, particle-based transfection, or other hybrid methods. In some embodiments, chemical-based transfection methods include using calcium phosphate, cyclodextrin, cationic polymers such as DEAE-dextran or polyethylenimine, cationic liposomes, or dendrimers. In some embodiments, non-chemical-based transfection methods include using electroporation, cell squeezing, sonoporation, optical transfection, protoplast fusion, impalefection, or hydrodynamic delivery. In some embodiments, particle-based transfection methods include using a gene gun where the nucleic acid is conjugated to an inert solid nanoparticle such as gold, magnetofection, carbon nanofibers or silicon nanowires functionalized with the nucleic acid molecules, or particle bombardment. In some embodiments, other hybrid transfection methods include nucleofection.

In some embodiments, the nucleic acid molecule is deoxyribonucleic acid (DNA) or ribonucleic acid (RNA). In some embodiments, the RNA molecule is a small RNA. In some embodiments, examples of small RNA include micro-RNA (miRNA), ribosomal RNA (rRNA), small nuclear RNA (snRNA), transfer RNA (tRNA), small nucleolar RNA (snoRNA), Piwi-interacting RNA (piRNA), tRNA-derived small RNA (tsRNA), small rDNA-derived RNA (srRNA), or 5S RNA. In some embodiments, the RNA molecule is a long RNA. In some embodiments, examples of long RNA include long non-coding RNA (lncRNA) or messenger RNA (mRNA). In some embodiments, the RNA molecule is a double stranded RNA (dsRNA), a circular RNA, a small interfering RNA (siRNA), antisense RNA (aRNA), cis-natural antisense transcript (cis-NAT), CRISPR RNA (crRNA), short hairpin RNA (shRNA), trans-acting siRNA (tasiRNA), repeat associated siRNA (rasiRNA), 7SK RNA (7SK), or enhancer RNA (eRNA).

In some embodiments, the nucleic acid molecule that partially, substantially, or completely deletes, silences, inactivates, or down-regulates an unwanted protein or amino acid sequence (e.g., SIRPα or ITIM) or alloantigen is introduced into a macrophage or a monocyte using a viral vector, such as retrovirus-based vector, adenovirus-based vector, lentivirus-based vector, or adeno-associated virus-based vector. In some embodiments, a nucleic acid molecule that partially, substantially, or completely enhances, activates, or up-regulates a gene encoding a receptor that binds to an opsonin is introduced into an isolated macrophage or monocyte using a viral vector, such as retrovirus-based vector, adenovirus-based vector, lentivirus-based vector, or adeno-associated virus-based vector.

### Methods of Treating Diseases

Disclosed herein, in certain embodiments, are methods of treating a pathogenic infection in an individual in need thereof, comprising: administering innate immune cells produced by any method described herein. Disclosed herein, in certain embodiments, are methods of treating a pulmonary disease in an individual in need thereof comprising: administering innate immune cells produced by any method described herein. Disclosed herein, in certain embodiments, are methods of treating an inflammatory disease in an individual in need thereof comprising administering innate immune cells produced by any method described herein. Disclosed herein, in certain embodiments, are methods of treating an autoimmune disease in an individual in need thereof comprising: administering innate immune cells produced by any method described herein. Disclosed herein, in certain embodiments, are methods of treating an immunodeficiency in an individual in need thereof comprising: administering innate immune cells produced by any method described herein. Disclosed herein, in certain embodiments, are methods of inducing or enhancing efferocytosis in an individual in need thereof comprising: administering innate immune cells produced by any method described herein. Disclosed herein, in certain embodiments, are methods of vaccinating an individual in need thereof comprising: administering to the individual (a) an isolated antigen or isolated allergen, and (b) innate immune cells produced by any method described herein. Disclosed herein, in certain embodiments, are methods of treating a complicated intra-abdominal infection (cIAI) in an individual in need thereof comprising: administering innate immune cells produced by any method described herein.

In some embodiments, the innate immune cells comprise macrophages. In some embodiments, the macrophages are obtained by differentiating monocytes that are isolated from a blood sample, an apheresis sample, or a bone marrow sample. In some embodiments, the macrophages are obtained by differentiating macrophage progenitor cells that are isolated from a blood sample, an apheresis sample, or a bone marrow sample. In some embodiments, the macrophage progenitor cells are hematopoietic stem cells, CD34+ stem cells, common myeloid progenitor cells, granulocyte-monocyte progenitor cells, or monocytes. In some embodiments, the macrophages are isolated from a human tissue sample. In some embodiments, the macrophages are isolated from a human peritoneal fluid sample. In some embodiments, the macrophages are derived from pluripotent cells. In some embodiments, the macrophages are obtained by differentiating embryonic stem cells (ESCs) into macrophage progenitor cells and further differentiating the macrophage progenitor cells into macrophages. In some embodiments, the macrophages are obtained by genetically reprogramming somatic cells into induced pluripotent stem cells (iPSCs) and differentiating iPSCs into macrophages. In some embodiments, the macrophages are Kupffer cells, histiocytes, alveolar macrophages, splenic macrophages, peritoneal macrophages, placental macrophages, osteoclasts, adipose tissue macrophage (ATM), or sinusoidal lining cells.

In some embodiments, the innate immune cells comprise monocytes. In some embodiments, the monocytes are isolated from a peripheral blood sample, a cord blood sample, an apheresis sample, or a bone marrow sample. In some embodiments, the monocytes are obtained by differentiating monocyte progenitor cells that are isolated from a blood sample, an apheresis sample, or a bone marrow sample. In some embodiments, the monocyte progenitor cells are hematopoietic stem cells, CD34+ stem cells, common myeloid progenitor cells, or granulocyte-monocyte progenitor cells. In some embodiments, the monocytes are derived from pluripotent cells. In some embodiments, the monocytes are obtained by differentiating embryonic stem cells (ESCs) into monocyte progenitor cells and further differentiating the monocyte progenitor cells into monocytes. In some embodiments, the monocytes are obtained by genetically reprogramming somatic cells into induced pluripotent stem cells (iPSCs) and differentiating iPSCs into monocytes.

In some embodiments, the innate immune cells are activated ex vivo before administration to the individual. In some embodiments, the innate immune cells are activated in vivo following administration to the individual, e.g., by the immune system of the individual and the presence of the unwanted pathogen. In some embodiments, the innate immune cells are activated in vivo following administration to the individual, e.g., by the immune system of the individual and the presence of a symbiotic pathogen.

In some embodiments, the innate immune cells are autologous. In some embodiments, the innate immune cells are allogenic.

In some embodiments, the innate immune cells are fresh, i.e., not frozen or previously frozen. In some embodiments, the innate immune cells are cryopreserved (frozen). In some embodiments, the innate immune cells are frozen and stored for later use (for example to facilitate transport). In some embodiments, the frozen innate immune cells are administered to the individual after being thawed.

In some embodiments, the innate immune cells are activated before administration to the individual. In some embodiments, the macrophages are not activated before administration to the individual. In some embodiments, the macrophages are activated by the immune system of the individual and the presence of the unwanted pathogen in the individual. In some embodiments, the macrophages are activated by the immune system of the individual and the presence of a symbiotic pathogen in the individual. In some embodiments, macrophages are co-administered with one or more compounds that activate the macrophages. For example, the macrophages are co-administered with phorbol myristate acetate, lipopolysaccharide (LPS), IFNγ, tumor-necrosis factor (TNF), IL-4, IL-13, or any combinations thereof.

In some embodiments, the individual is administered a pre-treatment with opsonins prior to administration of the innate immune cells. Exemplary opsonins for use with the methods described herein include, but are not limited to an antibody, a complement protein, or a circulating protein. In some embodiments, the antibody has an immunoglobulin G (IgG) or IgA isotype. In some embodiments, the complement protein is C3b, C4b, C5, or C1q. In some embodiments, the circulating protein is a pattern recognition receptor (PRR), pentraxin, collectin, or ficolin. In some embodiments, the individual is administered a dose of an IgG antibody, an IgA antibody, C3b, C4b, C5, C1q, pentraxin, collectin, ficolin, or combinations thereof, prior to the administration of the innate immune cells.

### Pathogenic Diseases

In some embodiments, the innate immune cells are administered to the individual following diagnosis of a pathogenic infection. In some embodiments, the pathogenic infection is a viral infection. In some embodiments, the pathogen infection is a bacterial infection. In some embodiments, the pathogenic infection is a fungal infection. In some embodiments, the pathogenic infection is a parasitic infection. In some embodiments, the pathogenic infection is a complicated intra-abdominal infection (cIAI).

In some embodiments, the innate immune cells are administered to the individual to prophylactically, for example if an individual is expected to be exposed to a pathogen. In some embodiments, the pathogen is a viral pathogen. In some embodiments, the pathogen is a bacterial pathogen. In some embodiments, the pathogen is a fungal pathogen. In some embodiments, the pathogen is a parasite.

In some embodiments, the pathogenic infection is a bacterial infection. In some embodiments, the pathogenic infection is a viral infection. In some embodiments, the pathogenic infection is a fungal infection. In some embodiments, the pathogenic infection is a parasitic infection.

In some embodiments, the pathogenic infection is a bacterial infection. In some embodiments, the bacterial infection is characterized by extracellular bacteria. In some embodiments, the bacterial infection is characterized by intracellular bacteria. In some embodiments, the bacterial infection is characterized by gram negative bacteria. In some embodiments, the bacterial infection is characterized by gram positive bacteria. In some embodiments, the bacterial infection is characterized by aerobic bacteria. In some embodiments, the bacterial infection is characterized by anaerobic bacteria.

In some embodiments, the bacteria are multi-drug resistant (MDR) bacteria, extensively drug resistant (XDR) bacteria, or pan-drug resistant (PDR) bacteria. In some embodiments, the term “multi-drug resistant bacteria” refers to bacteria that are resistant to one key antimicrobial agent. In some embodiments, the term “extensively-drug resistant bacteria” refers to bacteria that are resistant to multiple antimicrobial agents and also likely to be resistant to all, or almost all, approved antimicrobial agents. In some embodiments, the term “extensively-drug resistant bacteria” refers to bacteria that are resistant to multiple antimicrobial agents and also likely to be resistant to all, or almost all, antimicrobial agents. In some embodiments, the term “pan-drug resistant bacteria” refers to bacteria that are resistant to all antimicrobial agents. In some embodiments, the drug is an antibiotic. In some embodiments, the pathogenic infection is characterized by antibiotic resistant bacteria. In some embodiments, the antibiotic is penicillin, ampicillin, carbapenem, fluoroquinolone, cephalosporin, tetracycline, erythromycin, methicillin, gentamicin, vancomycin, imipenem, ceftazidime, levofloxacin, linezolid, daptomycin, ceftaroline, clindamycin, or ciprofloxacin. In some embodiments, the antibiotic is a first, a second, a third, a fourth, a fifth, a sixth, a seventh, an eighth, a ninth, or a tenth generation antibiotic.

In some embodiments, the bacterial infection is characterized by the presence of one or more of the following bacterial genera: Klebsiella, Clostridium, Naegleria, Acinetobacter, Bacteroides, Borrelia, Brucella, Burkholderia, Campylobacter, Ehrlichia, Enterobacteriaceae, Enterococcus, Escherichia, Haemophilus, Helicobacter, Fusobacterium, Leptospira, Listeria, Mycobacterium, Mycoplasma, Neisseria, Nocardia, Prevotella, Rickettsia, Salmonellae, Shigella, Staphylococcus, Streptococcus, Stenotrophomonas, Lactobacillus, Corynebacterium, Morganella, Proteus, Enterobacter, and Treponema. In some embodiments, the pathogenic infection is characterized by bacteria including: Klebsiella pneumoniae, Klebsiella oxytoca, Clostridium difficile, Naegleria fowleri, Acinetobacter baumannii, Borrelia burgdorferi, Escheririchia coli, Haemophilus influenza, Listeria monocytogenes, Mycobacterium tuberculosis, Neisseria meningitidis, Nocardia asteroids, Staphylococcus aureus, Streptococcus agalactiae, Streptococcus intermedius, Streptococcus pneumoniae, Treponema pallidum, Enterococcus faecium, Enterococcus faecalis Helicobacter pylori, Neisseria gonorrhoeae, Streptococcus pneumoniae, Shigella spp., Burkholderia cepacia, Mycobacterium tuberculosis, Serratia, Stenotrophomonas maltophilia, Lactobacillus, Peptostreptococcus, Staphylococcus epidermidis, Enterococcus, Enterobacter, Proteus, gram positive anaerobic cocci (GPAC), Bacteroides fragilis, Proteus mirabilis, Morganella morganii, Bacteroides resistant to metronidazole, and non-tuberculous mycobacteria.

In some embodiments, the bacterial infection comprises a biofilm. As used herein, the term “biofilm” means a group of microbial cells that irreversibly adhere to each other and to a surface and are enclosed within an extracellular polymeric substrate (EPS) composed mainly of a polysaccharide material.

In some embodiments, the pathogenic infection is a viral infection. In some embodiments, the virus is a DNA virus or an RNA virus. In some embodiments, the viral infection is characterized by the presence of one or more of the following virial families including: Bunyaviridae, Flaviviridae, Herpesviridae, Orthomyxoviridae, Papovaviridae, Paramyxoviridae, Picornaviridae, Togaviridae, Retroviridae, and Rhabdoviridae. In some embodiments, the viral infection is characterized by a virus including: Herpes simplex virus (HSV), varicella zoster virus, cytomegalovirus (CMV), Epstein-Barr virus (EBV), Eastern equine encephalitis (EEE), western equine encephalitis (WEE), rubella virus, poliovirus, coxsackievirus, an enterovirus, St. Louis encephalitis (SLE), Japanese encephalitis, rubeola (measles) virus, mumps virus, California encephalitis, LaCrosse virus, human immunodeficiency virus (HIV), rabies virus, and Influenza A virus.

In some embodiments, the pathogenic infection is a parasitic infection. In some embodiments, a macrophage activated in vitro by exposure to IL-4 and/or IL-13 is administered as a method to treat a parasitic infection. In some embodiments, the parasite is a helminth or a protozoan. In some embodiments, the parasitic infection is characterized by the presence of one of the following parasite genera comprising: Angiostrongylus, Cysticercus, Echinococcus, Entamoeba, Gnathostoma, Paragnoimus, Plasmodium, Taenia, Toxoplasma, Trypanosoma, and Schistosoma. In some embodiments, the pathogenic infection is characterized by a parasite including: Angiostrongylus cantonesis, Entamoeba histolytica, Gnathostoma spinigerum, Taenia solium, Toxoplasma gondii, and Trypanosoma cruzi.

In some embodiments, the pathogenic infection is a fungal infection. In some embodiments, the fungal infection is characterized by the presence of one or more of the following fungal genera comprising: Aspergillus, Bipolaris, Blastomyces, Candida, Cryptococcus, Coccidioides, Curvularia, Exophiala, Histoplasma, Mucorales, Ochroconis, Pseudallescheria, Ramichloridium, Sporothrix, Zygomyctes, Pneumocystis, and Trichosporon. In some embodiments, the pathogenic injection is characterized by a fungus including Blastomyces dermatitidis, Candida albicans, Candida glabrata, Coccidioides immitis, Cryptococcus gattii, Cryptococcus neoformans, Curvalaria pallescens, Exophiala dermatitidis, Histoplasma capsulatum, Onchroconis gallopava, Psudallescheria boydii, Ramichloridium mackenziei, Sporothrix schenckii, Aspergillus fumigatus, Candida parapsilosis, Coccidioides neoformans, Pneumocystis carinii, and Trichosporon asahii. In some embodiments, the fungal infection is characterized by the presence of Aspergillus fungi. In some embodiments, the fungal infection is characterized by the presence of Candida fungi. In some embodiments, the fungal infection is characterized by antifungal resistant fungi. In some embodiments, the pathogenic infection is characterized by antifungal resistant fungi. In some embodiments, the antifungal is fluconazole, itraconazole, voriconazole, posaconazole, isavuconazole, anidulafungin, caspofungin, micafungin, or any combination thereof.

In some embodiments, the pathogenic infection is a hospital acquired infection (HAI) or a nosocomial infection. In some embodiments, the HAI is selected from: a catheter-line associated infection, a catheter-related bloodstream infection, a central line bloodstream infection, a catheter-associated urinary tract infection, or ventilator associated pneumonia. In some embodiments, the central line bloodstream infection is an infection that occurs when bacteria or viruses enter the bloodstream through a central line. In some embodiments, a central line is a catheter or tube that is placed in fluidic connection with the bloodstream via an opening through a large vein in the neck, groin, or chest. In some embodiments, the central line is a central venous catheter.

In some embodiments, the pathogenic infection is selected from sepsis, a urinary tract infection, pneumonia, staphylococcal food poisoning, typhoid fever, vibrio enteritis, viral pneumonia, yellow fever, candidiasis, cholera, botulism, Clostridium difficile colitis, gas gangrene, food poisoning by Clostridium perfringens, tetanus, granuloma inguinale (donovanosis), primary amoebic meningoencephalitis (PAM), lyme disease, brucellosis, hemolytic-uremic syndrome, chancroid, Haemophilus influenzae infection, leptospirosis, listeriosis, buruli ulcer, leprosy, mycoplasma pneumonia, gonorrhea, meningococcal disease, neonatal conjunctivitis, nocardiosis, prevotella infection, epidemic typhus, rickettsial infection, rickettsial pox, Rocky Mountain spotted fever, typhus fever, cellulitis, or syphilis.

In some embodiments, the pathogenic infection is an infection associated with combat-related injuries. Non-limiting examples of combat-related injuries include extremity trauma, extremity injuries, musculoskeletal injuries, soft tissue wounds, abdominal injuries, traumatic extremity amputations, traumatic lacerations, gunshot wounds, injuries caused by explosions, thoracic trauma, skin injuries, facial injuries, brain injuries, and/or gastrointestinal injuries.

In some embodiments, the pathogenic infection is a chronic wound infection. A chronic wound is a wound that does not heal within an average time frame (e.g. three months) and does not follow the typical wound healing stages (e.g. the wound persists in an inflammatory state for an extended period of time). Chronic wounds are caused by a variety of factors including, but not limiting to ischemia, reperfusion injury, bacterial colonization, poor circulation, neuropathy, difficulty moving, systemic illnesses, repeated trauma (e.g. subcutaneous administration of heroin by heroin users), age, vasculitis, immune suppression, pyoderma gangrenosum, ischemic diseases, long term medical drug usage (e.g. steroids), cancer (e.g. squamous cell carcinoma), chronic fibrosis, edema, sickle cell disease, and/or peripheral artery disease (e.g. caused by atherosclerosis). In some embodiments, the chronic wound is a venous ulcer, a diabetic chronic wound, a pressure ulcer, a radiation poisoning wound, and/or ischemia.

In some embodiments, bacterial colonization causes a wound to become a chronic wound. In some embodiments, patients with chronic wound infections develop drug resistant bacterial strains. In some embodiments, patients with chronic wound infections carry methicillin-resistant Staphylococcus aureus. In some embodiments, patients with chronic wound infections carry multi-drug resistant bacteria, extensively drug resistant bacteria, or pan-drug resistant bacteria.

### Complicated Intra Abdominal Infections

In some embodiments, the pathogenic infection is a complicated intra-abdominal infection (cIAI). In some embodiments, the cIAI is a bacterial infection. In some embodiments, the cIAI is a fungal infection. In some embodiments, the cIAI is characterized by bacteria including: Lactobacillus, Klebsiella pneumoniae, Klebsiella pneumoniae resistant to third generation cephalosporin, Klebsiella oxytoca, Klebsiella oxytoca resistant to third generation cephalosporin, Clostridium, Clostridium difficile, Acinetobacter baumannii, Escherichia coli, Escherichia coli resistant to third generation cephalosporin, Pseudomonas, Pseudomonas aeruginosa, Staphylococcus aureus, Streptococcus spp., Streptococcus pyogenes, Enterobacteriaceae, Enterococcus faecium, Enterococcus faecalis, Helicobacter pylori, Streptococcus pneumoniae, Streptococcus agalactiae, Serratia, Stenotrophomonas maltophilia, Corynebacterium, Peptostreptococcus, Peptococcus, Staphylococcus epidermidis, Enterococcus, Enterobacter, Proteus, gram-positive anaerobic cocci (GPAC), Bacteroides fragilis, Proteus mirabilis, Bacteroides, Bacteroides resistant to metronidazole, and Morganella morganii. In some embodiments, the cIAI is characterized by the presence of a fungus such as Candida, Candida albicans, Candida albicans resistant to fluconazole, non-albicans Candida, non-albicans Candida resistant to fluconazole, or Candida glabrata. In some embodiments, the cIAI is a hospital-acquired complicated intra-abdominal infection (cIAI).

Intra-abdominal infections are categorized as either uncomplicated intra-abdominal infections or complicated intra-abdominal infections (cIAI), depending on their degree of invasiveness. cIAI extend beyond the source organ into the peritoneal space and are associated with systemic symptoms including, but not limited to, fever; tachycardia; tachypnea; hypotension; local, referred, generalized, or absent abdominal pain; anorexia; nausea; vomiting; diarrhea; abdominal fullness; distension; obstipation; shock; acidosis; extra-abdominal organ failure; or any combination thereof. In contrast to uncomplicated intra-abdominal infections, cIAIs are characterized by the need for surgical and/or radiological drainage procedures in addition to antimicrobial therapies. The human body's defensive response against cIAI includes lymphatic clearance, phagocytosis, sequestration of fibrin, and/or anatomical localization. In some embodiments, cIAIs arise downstream of and/or are associated with a variety of conditions including, but not limited to, postoperative abdominal infection, colorectal surgery, abdominal surgery, appendectomy, cholecystectomy, hysterectomy, hernia surgery, exploratory laparotomy, colectomy, ostomy, ileostomy, bowel resection, proctolectomy, strictureplasty, appendicitis, appendicitis with perforation, appendicitis with periappendiceal abscess, gangrenous appendicitis, acute appendicitis, intra-abdominal sepsis, peritonitis, florid fecal peritonitis, diffuse peritonitis, localized peritonitis, intra-abdominal abscesses, abdominal surgery, gastrointestinal perforation such as perforation of the stomach, perforation of the intestines, perforation of the gallbladder, perforation of the appendix, gastroduodenal perforation, small bowel perforation, colonic non-diverticular perforation, and/or post-traumatic perforation, ruptured appendix, ruptured diverticulum, cholecystitis, cholecystitis with perforation, cholecystitis with abscess, diverticulitis, diverticulitis with perforation, diverticulitis with abscess, peptic ulcer disease, gastric ulcer, duodenal ulcer, stomach ulcer, phlegmon, pancreatitis, colitis, bowel obstruction, Clostridium difficile colitis, abdominal compartment syndrome, hepatic abscess, mesenteric ischemia, post-operative peritonitis, primary peritonitis, secondary peritonitis, tertiary peritonitis, peritoneal dialysis-associated peritonitis, spontaneous bacterial peritonitis, gangrene, pancreatitis, trauma to the abdomen, pelvic inflammatory disease, Chron's disease, or any combination thereof. In some embodiments, the pathogenic infection is a hospital-acquired complicated intra-abdominal infection (cIAI). In some embodiments, the pathogenic infection is polymicrobial. In some embodiments, the cIAI is polymicrobial.

### Pulmonary Diseases

In some embodiments, the pulmonary disease is associated with a pathogenic infection. In some embodiments, the pulmonary disease is a chronic pulmonary disease. In some embodiments, the pulmonary disease is an acute pulmonary disease. In some embodiments the pulmonary disease is chronic obstructive pulmonary disease (COPD), chronic obstructive airway disease (COAD), acute bronchitis, chronic bronchitis, emphysema, pulmonary emphysema, asthma, cystic fibrosis, allergic sinusitis, pulmonary hypertension, pneumonia, tuberculosis, pulmonary edema, pneumoconiosis, interstitial lung disease, sarcoidosis, idiopathic pulmonary fibrosis, pleural effusion, pneumothorax, mesothelioma, acute respiratory distress syndrome (ARDS), alpha-1 antitrypsin deficiency, asbestosis, bronchiectasis, bronchiolitis, bronchiolitis obliterans with organizing pneumonia (BOOP), bronchopulmonary dysplasia, byssinosis, chronic thromboembolic pulmonary hypertension (CTEPH), coccidioidomycosis, cryptogenic organizing pneumonia (COP), hantavirus pulmonary syndrome (HPS), histoplasmosis, human metapneumovirus (hMPV), hypersensitivity pneumonitis, influenza, lung cancer, lymphangioleiomyomatosis (LAM), middle eastern respiratory syndrome (MERS), nontuberculosis mycobacteria, pertussis, primary ciliary dyskinesia (PCD), pulmonary arterial hypertension (PAH), pulmonary fibrosis (PF), respiratory syncytial virus (RSV), severe acute respiratory syndrome (SARS), or silicosis.

### Inflammatory Diseases

In some embodiments, the inflammatory disease is a chronic inflammatory disease. In some embodiments, a macrophage activated in vitro by exposure to IL-4 and/or IL-13 is administered as a method to treat a chronic inflammatory disease. In some embodiments, the chronic inflammatory disease is atherosclerosis. In some embodiments, the chronic inflammatory disease is lupus. In some embodiments, the chronic inflammatory disease is rheumatoid arthritis. In some embodiments, the chronic inflammatory disease is type 1 diabetes. In some embodiments, the inflammatory disease includes osteoarthritis, psoriatic arthritis, Crohn's disease, colitis, dermatitis, diverticulitis, fibromyalgia, hepatitis, irritable bowel syndrome (IBS), systemic lupus erythematous (SLE), nephritis, Alzheimer's disease, Parkinson's disease, ulcerative colitis, cardiovascular disease, acne vulgaris, celiac disease, chronic prostatitis, diverticulitis, glomerulonephritis, hidradenitis suppurativa, interstitial cystitis, inflammatory bowel disease, otitis, pelvic inflammatory disease, reperfusion injury, rheumatic fever, transplant rejection, vasculitis, allergies and resulting hypersensitivities, myopathies such as systemic sclerosis, dermatomyositis, polymyositis, or inclusion body myositis, leukocyte defects such as Chediak-Higashi syndrome, chronic granulomatous disease, cancer-related inflammation, HIV and AIDS, or obesity.

### Autoimmune Diseases

In some embodiments, the autoimmune disease is rheumatoid arthritis. In some embodiments, the autoimmune disease is lupus. In some embodiments, the autoimmune disease is type 1 diabetes. In some embodiments, the autoimmune disease is acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmunedysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal osteomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophilic fasciitis, erythema nodosum, experimental allergic encephalomyelitis, Evan's syndrome, fibromyalgia, fibrosing alveolitis, giant cell arteritis (temporal arteritis), glomerulonephritis, Good pasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), Lupus (SLE), Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II & III autoimmune polyglandular syndrome, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, dilated cardiomyopathy, myocarditis, autoimmune polyendocrine syndrome type I (APS-I), cystic fibrosis vasculitis, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjögren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuropathy, paraneoplastic pemphigus, paraneoplastic stiff man syndrome, paraneoplastic encephalomyelitis, subacute autonomic neuropathy, cancer-associated retinopathy, paraneoplastic opsoclonus-myoclonus ataxia, lower motor neuron syndrome, and Lambert-Eaton myasthenic syndrome.

### Immunodeficiency

In some embodiments, the individual is immunodeficient due to administration of chemotherapeutic agents. In some embodiments, the individual is immunodeficient due to radiation therapy. In some embodiments, the individual is immunodeficient due to an autoimmune disease. In some embodiments, the individual is immunodeficient due to senility or old age. In some embodiments, the individual is immunodeficient and susceptible to acquire a disease described herein. In some embodiments, the individual acquired a disease described herein due to an immunodeficiency.

### Efferocytosis

Efferocytosis is the process by which phagocytes remove apoptotic or necrotic cells. Apoptotic cells actively recruit phagocytes by secreting chemotaxins, shifting their surface glycoprotein composition, changing the basal asymmetry of their lipid membranes, and displaying specific molecules on their surface such as phosphatidylserine (PS). Efferocytosis contributes to anti-inflammatory and tolerogenic processes, favoring tissue repair and suppressing inflammation. Impaired efferocytosis contributes to secondary necrosis, sustained inflammation, and/or autoimmunity provoked by release of pro-inflammatory cell contents during cell necrosis. Improper clearance of apoptotic cells contributes to the establishment and progression of certain diseases such as inflammatory diseases, autoimmune diseases, pulmonary diseases including asthma, COPD, and cystic fibrosis, obesity, type 2 diabetes, and atherosclerosis. Thus, increasing or enhancing efferocytosis provides a method of treating or preventing an inflammatory disease, an autoimmune disease, or a pulmonary disease in a subject in need thereof.

In some embodiments, the individual has an inflammatory disease. In some embodiments, the individual has an autoimmune disease. In some embodiments, the individual has a neurodegenerative disease. Exemplary neurodegenerative diseases include, but are not limited to multiple sclerosis, Parkinson's disease, Alzheimer's disease, dementia, Huntington's disease, amyotrophic lateral sclerosis, and Batten disease. In some embodiments, the individual has asthma. In some embodiments, the individual has rheumatoid arthritis. In some embodiments, the individual has atherosclerosis the individual has. In some embodiments, the individual has COPD. In some embodiments, the individual has pulmonary fibrosis.

### Vaccine Adjuvant

In some embodiments, innate immune cells are administered to the individual before, after, or simultaneously with an isolated antigen or isolated allergen. In some embodiments, the innate immune cells are administered in the same dosage form as the isolated antigen or allergen. In some embodiments, the isolated antigen or the isolated allergen is expressed by the innate immune cell. In some embodiments, the isolated antigen or the isolated allergen is expressed by the macrophage. In some embodiments, the isolated antigen or the isolated allergen is expressed by the monocyte. In some embodiments, the innate immune cells are engineered to express the antigen or allergen. In some embodiments, the innate immune cells are administered as a vaccine adjuvant. In some embodiments, the individual being administered the innate immune cells as a vaccine adjuvant lacks an effective innate immune response. In some embodiments, the individual being administered the innate immune cells as a vaccine adjuvant is an elderly individual. In some embodiments, the innate immune cells increase vaccine efficiency when administered as a vaccine adjuvant.

### Combination Therapies

Disclosed herein, in certain embodiments, are methods of treating a pathogenic infection in an individual in need thereof, comprising: administering innate immune cells produced by any method described herein, and an additional therapeutic agent. Disclosed herein, in certain embodiments, are methods of treating a pulmonary disease in an individual in need thereof comprising: administering innate immune cells produced by any method described herein, and an additional therapeutic agent. Disclosed herein, in certain embodiments, are methods of treating an inflammatory disease in an individual in need thereof comprising administering innate immune cells produced by any method described herein, and an additional therapeutic agent. Disclosed herein, in certain embodiments, are methods of treating an autoimmune disease in an individual in need thereof comprising: administering innate immune cells produced by any method described herein, and an additional therapeutic agent. Disclosed herein, in certain embodiments, are methods of treating an immunodeficiency in an individual in need thereof comprising: administering innate immune cells produced by any method described herein, and an additional therapeutic agent.

In some embodiments, the innate immune cells comprise macrophages. In some embodiments, the macrophages are obtained by differentiating monocytes that are isolated from a blood sample, an apheresis sample, or a bone marrow sample. In some embodiments, the macrophages are obtained by differentiating macrophage progenitor cells that are isolated from a blood sample, an apheresis sample, or a bone marrow sample. In some embodiments, the macrophage progenitor cells are hematopoietic stem cells, CD34+ stem cells, common myeloid progenitor cells, granulocyte-monocyte progenitor cells, or monocytes. In some embodiments, the macrophages are isolated from a human tissue sample. In some embodiments, the macrophages are isolated from a human peritoneal fluid sample. In some embodiments, the macrophages are derived from pluripotent cells. In some embodiments, the macrophages are obtained by differentiating embryonic stem cells (ESCs) into macrophage progenitor cells and further differentiating the macrophage progenitor cells into macrophages. In some embodiments, the macrophages are obtained by genetically reprogramming somatic cells into induced pluripotent stem cells (iPSCs) and differentiating iPSCs into macrophages. In some embodiments, the macrophages are Kupffer cells, histiocytes, alveolar macrophages, splenic macrophages, placental macrophages, peritoneal macrophages, osteoclasts, adipose tissue macrophage (ATM), or sinusoidal lining cells.

In some embodiments, the innate immune cells comprise monocytes. In some embodiments, the monocytes are isolated from a peripheral blood sample, a cord blood sample, an apheresis sample, or a bone marrow sample. In some embodiments, the monocytes are obtained by differentiating monocyte progenitor cells that are isolated from a blood sample, an apheresis sample, or a bone marrow sample. In some embodiments, the monocyte progenitor cells are hematopoietic stem cells, CD34+ stem cells, common myeloid progenitor cells, or granulocyte-monocyte progenitor cells. In some embodiments, the monocytes are derived from pluripotent cells. In some embodiments, the monocytes are obtained by differentiating embryonic stem cells (ESCs) into monocyte progenitor cells and further differentiating the monocyte progenitor cells into monocytes. In some embodiments, the monocytes are obtained by genetically reprogramming somatic cells into induced pluripotent stem cells (iPSCs) and differentiating iPSCs into monocytes.

In some embodiments, the innate immune cells are activated ex vivo before administration to the individual. In some embodiments, the innate immune cells are activated in vivo following administration to the individual, e.g., by the immune system of the individual and the presence of the unwanted pathogen. In some embodiments, the innate immune cells are activated in vivo following administration to the individual, e.g., by the immune system of the individual and the presence of a symbiotic pathogen.

In some embodiments, the innate immune cells are autologous. In some embodiments, the innate immune cells are allogenic.

In some embodiments, the innate immune cells are fresh, i.e., not frozen or previously frozen. In some embodiments, the innate immune cells are frozen and stored for later use (for example to facilitate transport). In some embodiments, the frozen innate immune cells are administered to the individual after being thawed.

In some embodiments, the innate immune cells are activated before administration to the individual. In some embodiments, the macrophages are not activated before administration to the individual. In some embodiments, the macrophages are activated by the immune system of the individual and the presence of the unwanted pathogen in the individual. In some embodiments, the macrophages are activated by the immune system of the individual and the presence of a symbiotic pathogen in the individual. In some embodiments, macrophages are co-administered with one or more compounds that activate the macrophages. For example, the macrophages are co-administered with phorbol myristate acetate, lipopolysaccharide (LPS), IFNγ, tumor-necrosis factor (TNF), IL-4, IL-13, or any combinations thereof.

In some embodiments, a method of treating a disease or condition in an individual in need thereof, comprises: administering macrophages produced by any method described herein, and an additional therapeutic agent. In some embodiments, a method of treating a disease or condition in an individual in need thereof, comprise: administering monocytes produced by any method described herein, and an additional therapeutic agent. In some embodiments, the additional therapeutic agent is selected from a group comprising: an antibiotic agent, an anti-inflammatory agent, an anti-allergy agent, a chemotherapeutic, an immunosuppressive agent, an immunostimulant agent, a respiratory agent, a macrophage activator, a monocyte activator, an immune cell, and/or a combination thereof. In some embodiments, the innate immune cell is conjugated to the additional therapeutic agent. In some embodiments, the macrophage is conjugated to the additional therapeutic agent. In some embodiments, the monocyte is conjugated to the additional therapeutic agent.

### Antibiotic Agents

In some embodiments, the additional therapeutic agent is an antibiotic agent, an antibacterial agent, an antiviral agent, an antifungal agent, or an anti-parasitic agent.

In some embodiments, antibacterial agent is selected from the group consisting of: ceftobiprole, ceftaroline, clindamycin, dalbavancin, daptomycin, linezolid, mupirocin, oritavancin, tedizolid, telavancin, tigecycline, vancomycin, an antibiotic agent belonging to the aminolylcosides class of antibiotics, an antibiotic agent belonging to carbapenems class of antibiotics, ceftazidime, cefepime, ceftobiprole, an antibiotic agent belonging to the fluoroquinolones class of antibiotics, piperacillin, tazobactam, ticarcillin, clavulanic acid, linezolid, an antibiotic agent belonging to the class of streptogramins class of antibiotics, tigecycline, daptomycin, or any combinations thereof.

In some embodiments, antiviral agent is selected from the group consisting of: abacavir, acyclovir, adefovir, amantadine, amprenavir, ampligen arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliever, faciclovir, fomivirsen, fosamprenavir, foscarnet, fofonet, fusion inhibitor, ganciclovir, ibacitabine, imunovir, idoxuridine, imiquimod, indinavir, inosine, integrase inhibitor, interferon type I, interferon type II, interferon type III, interferon, lamivudine, lopinavir, loviride, maraviroc, moroxydine, methisazone, nelfinavir, nevirapine, nexavir, nitazoxanide, nucleoside analogues, novir, oseltamivir, peginterferon alfa-2a, penciclovir, peramivir, pleconaril, podophyllotoxin, a protease inhibitor, raltegravir, a reverse transcriptase inhibitor, ribavirin, rimantadine, ritonavir, pyramidine, saquinavir, sofosbuvir, stavudine, an antiretroviral synergistic enhancer, telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, or any combinations thereof.

In some embodiments, an antifungal agent is antimycotic agent. In some embodiments, the antifungal agent is selected from the group consisting of: a polyene, imidazone, triazole, thiazole, allylamine, or echinocandin classes of antifungals. In some embodiments, the antifungal agent is selected from the group consisting of: benzoic acid, ciclopirox olamine, flucytosine, griseofulvin, haloprogin, tolnaftate, undecylenic acid, crystal viole, balsam of Peru, clotrimazole, econazole, miconazole, terbinafine, fluconazole, ketoconazole, amphotericin, itraconazole, posaconazole, isavuconazonium, voriconazole, caspofungin, anidulafungin, micafungin, griseofulvin, terbinafine, flucytosine, nystatin, amphotericin B lipid complex, amorolfin, butenafine, naftifine, abafungin, albaconazole, efinaconazole, epoxiconazole, isavuconazole, propiconazole, ravuconazole, terconazole, bifonazole, butoconazole, fenticonazole, luliconazole, omoconazole, oxiconazole, sertaconazole, sulconazole, tioconazole, candicin, filipin, hamycin natamycin, rimocidin, or any combinations thereof.

### Anti-Inflammatory Agents

In some embodiments, the additional therapeutic agent is an anti-inflammatory. In some embodiments, the anti-inflammatory is selected from the group consisting of: acetaminophen, a nonsteroidal anti-inflammatory drug (NSAID), a cyclooxygenase (COX)-1 inhibitor, a disease-modifying anti-rheumatic drug (DMARD), or a COX-2 inhibitor.

In some embodiments, the NSAID is bromfenac, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, nepafenac, oxaprozin, phenylbutazone, piroxicam, sulindac, tometin, or a combination thereof.

In some embodiments, the DMARD is hydroxychloroquine, sulfasalazine, leflunomide, methotrexate, minocycline, abatacept, adalimumab, anakinra, certolizumab, etanercept, etanercpt-szzs, golimumab, infliximab, rituximab, tocilizumab, azathioprine, tofacitinib, or a combination thereof. In some embodiments, the COX-1 inhibitor is sulindac sulfide, pravadoline, indomethacin, naproxen, meclofenamate sodium, ibuprofen, piroxicam, MK-886 sodium salt, (S)-ibuprofen, (S)-ketoprofen, (R)-ibuprofen, meloxicam, resveratrol, diclofenac sodium, flurbiprofen, aspirin, loganin, SC 560, fexofenadine HCl, pterostilbene, acetaminophen, FR 122047 HCl, tenisdap, cis-resveratrol, ketoprofen, ketorolac, NO-indomethacin, (S)-(+)-flurbiprofen, sedanolide, valeryl salicylate, licofelone, ampiroxicam, naproxen sodium salt, zaltoprofen, acetylsalicylic acid-d4, CAY10589, ZLJ-6, YS121, diclofenac diethylamine, TFAP, MEG HCl, or any combination thereof.

In some embodiments, the COX-2 inhibitor is celecoxib, 6-methoxy-2-naphthylacetic acid, acetylsalicylic acid-d4, N-(2-phenylethyl)indomethacin amide, N-(3-pyridyl)indomethacin amide, SC236, indomethacin heptyl ester, CAY10589, ZLJ-6, YS121, diclofenac diethylamine, MEG HCl, sulindac sulfide, pravadoline, naproxen, meclofenamate sodium, ibuprofen, piroxicam, (S)-ibuprofen, (S)-ketoprofen, (R)-ibuprofen, meloxicam, APHS, diclofenac sodium, flurbiprofen, fexofenadine HCl, pterostilbene, acetaminophen, etodolac, ketoprofen, ketorolac, NO-indomethacin, (S)-(+)-flurbiprofen, sedanolide, licofelone, N-(4-acetamidophenyl)indomethacin amide, ampiroxicam, zaltoprofen, valdecoxib, rofecoxib, celecoxib, or any combination thereof.

### Anti Allergy Agents

In some embodiments, the additional therapeutic is an anti-allergy agent. In some embodiments, an anti-allergy agent is an antihistamine, a glucocorticoid, epinephrine, a mast cell stabilizer, an antileukotriene agent, an anticholinergic, or a decongestant. In some embodiments, the antihistamine is an H₁-antihistamine, an H₂-antihistamine, an H₃-antihistamine, an H₄-antihistamine, or a histidine decarboxylase inhibitor. In some embodiments, the H₁-antihistamine is an H₁ antagonist or an H₁ inverse agonist. In some embodiments, the H₁ antagonists include acrivastine, azelastine, Benadryl, diphenhydramine, bilastine, bromodiphenhydramine, brompheniramine, buclizine, carbinoxamine, cetirizine, chlorodiphenhydramine, chlorphenamine, chlorpromazine, clemastine, cyclizine, cyproheptadine, dexbrompheniramine, dexchlorpheniramine, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, or any combinations thereof. In some embodiments, the H₁ inverse agonists include cetirizine, levocetirizine, desloratadine, pyrilamine, or any combinations thereof. In some embodiments, the H₂-antihistamines include cimetidine, famotidine, lafutidine, nizatidine, ranitidine, roxatidine, tiotidine, or any combinations thereof. In some embodiments, the H₃-antihistamines include clobenpropit, ABT-239, ciproxifan, conessine, A-349, A-821, and thioperamide. In some embodiments, the H₄-antihistamines include thioperamide, JNJ 7777120, and VUF-6002. In some embodiments, the histidine decarboxylase inhibitors include tritoqualine and catechin.

In some embodiments, the glucocorticoid is selected from the group comprising: alclometasone, AZD5423, beclometasone dipropionate, betamethasone dipropionate, budesonide, chlormadinone acetate, chloroprednisone, ciclesonide, corticosteroid ester, cortisol, cortisporin, cortivazol, cyproterone, cyproterone acetate, deflazacort, delmadinone acetate, 11-deoxycortisol, dexamethasone, 5α-dihydrocorticosterone, fludroxycortide, flugestone, flugestone acetate, flumetasone, flunisolide, fluocinonide, fluocortolone, fluorometholone, fluoxymesterone, fluticasone, fluticasone furoate, fluticasone propionate, gestodene, a glucocorticoid receptor modulator, hydrocortamate, hydrocortisone, 15β-hydroxycyproterone acetate, 17α-hydroxyprogesterone, corticosteroid esters, mapracorat, medrogestone, medroxyprogesterone acetate, medrysone, megestrol acetate, membrane glucocorticoid receptor, meprednisone, methylprednisolone, metribolone, mometasone, mometasone, furoate, norgestomet, osaterone acetate, otobiotic, paramethasone, prebediolone acetate, prednisolone, prednisone, prednylidene, pregnenolone acetate, pregnenolone succinate, proctosedyl, progesterone, progesterone, promegestone, quingestrone, rimexolone, RU-28362, segesterone acetate, tetrahydrocorticosterone, tetrahydrogestrinone, tixocortol, tobramycin/dexamethasone, triamcinolone, and ulobetasol.

In some embodiments, the mast cell stabilizer is selected from the group comprising: β2-adrenergic agonists, cromoglicic acid, cromoly, nedocromil, ketotifen, methylxanthines, olopatadine, omalizumab, pemirolast, quercetin, compound 13, R112, ER-27317, U63A05, WHI-131, hypothemycin, midostaurin, CP99994, K1, Ro 20-1724, fullerenes, siguazodan, vacuolin-1, CMT-3, OR-1384, OR-1958, TLCK, TPCK, bromoenol lactone, cerivastatin, atorvastatin, fluvastatin, and nilotinib.

In some embodiments, the antileukotriene agent is selected from the group comprising: montelukast, zafirlukast, zileuton, pranlukast, ZD-2138, Bay X 1005, and MK-0591.

In some embodiments, the anticholinergic is an antimuscarinic agent or an antinicotinic agent. In some embodiments, antimuscarinic agents are atropine, benzatropine, biperide, chlorpheniramine, dicyclomine, dimenhydrinate, diphenhydramine, doxepin, doxylamine, glycopyrrolate, ipratropium, orphenadrine, oxitropium, oxybutynin, tolterodine, tiotropium, a tricyclic antidepressant, tryhexypheniyl, scopolamine, solifenacin, tropicamide, or any combinations thereof. In some embodiments, antinicotinic agents are bupropion, dextromethorphan, doxacurium, hexamethonium, mecamylamine, and tubocurarine.

In some embodiments, the decongestant is selected from the group comprising: ephedrine, levomethamphetamine, naphazoline, oxymetazoline, phenylephrine, phenylpropanolamine, propylhexedrine, pseudoephedrine, synephrine, tetryzoline, tramazoline, xylometazoline, cafaminol, cyclopentamine, epinephrine, fenoxazoline, levonordefrin, mephentermine, metizoline, norepinephrine, tuaminoheptane, and tymazoline.

### Chemotherapeutics

In some embodiments, the additional therapeutic agent is a chemotherapeutic agent. In some embodiments, the innate immune cells are administered prophylactically in combination with the chemotherapeutic agent in order to treat an immunodeficiency caused by the chemotherapeutic agent. In some embodiments, the innate immune cells are administered in combination with the chemotherapeutic agent in order to treat an immunodeficiency caused by the chemotherapeutic agent. In some embodiments, the chemotherapeutic agent is an alkylating agent, an anthracycline, a cytoskeletal disruptor, an epothilone, a histone deacetylase inhibitor, a topoisomerase I inhibitor, a topoisomerase II inhibitor, a kinase inhibitor, a nucleotide analog, a precursor analog, a peptide antibiotic, a platinum-based agent, a retinoid, or a vinca alkaloid. In some embodiments, chemotherapeutic agents include: actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine, and vinorelbine.

### Immunosuppressive Agents

In some embodiments, the additional therapeutic agent is an immunosuppressive agent. In some embodiments, the immunosuppressive agent is a glucocorticoid, a cytostatic agent, an antibody, a drug acting on immunophilins, or a combination thereof. In some embodiments, a cytostatic agent inhibits cell division. In some embodiments, the cytostatic agent is an alkylating agent or an antimetabolite. In some embodiments, the alkylating agent is nitrogen mustard (cyclophosphamide), nitrosourea, or a platinum compound. In some embodiments, the antimetabolite is a folic acid analogue, such as methotrexate; a purine analogue, such as azathioprine and mercaptourine; a pyrimidine analogue, such as fluorouracil; a protein synthesis inhibitor; or a cytotoxic antibiotic, such as dactinomycin, anthracyclin, mitomycin C, bleomycin, or mithramycin. In some embodiments, the immunosuppressive agent is azathioprine, mycophenolate mofetil, cyclosporine, leflunomide, chlorambucil, or a combination thereof.

### Immunostimulants

In some embodiments, the additional therapeutic agent is an immunostimulant agent. In some embodiments, the immunostimulant agent is specific immunostimulant or a non-specific immunostimulant. In some embodiments, the specific immunostimulant is a vaccine, an antigen, or a combination thereof. In some embodiments, the non-specific immunostimulant is an adjuvant. In some embodiments, the immunostimulant is an endogenous immunostimulant, such as deoxycholic acid (DCA); a supplement, such as vitamin C, vitamin B6, vitamin A, and vitamin E; a synthetic immunostimulant, such as imiquimod and resiquimod; colony stimulating factors, such as filgrastim, pegfilgrastim, tbo-filgrastim, and sargramostim; interferons, such as interferon gamma, interferon beta, interferon alpha; interleukins, such as aldesleukin and oprelvekin; glatiramer; pegademase bovine; plerixafor; or any combination thereof.

### Respiratory Agents

In some embodiments, the additional therapeutic agent is a respiratory agent. In some embodiments, the respiratory agent is an antiasthmatic drug, a bronchodilator, a glucocorticoid, an antihistamine, an antitussive agent, a decongestant, an expectorant, a leukotriene modifier, a lung surfactant, a respiratory inhalant, a mast cell stabilizer, a corticosteroid, a mucolytic agent, a selective phosphodiesterase-4 inhibitor, an anti-IgE antibody, a leukotriene receptor antagonist, a respiratory stimulant, an oxygen antimicrobial, an antiviral, an expectorant. In some embodiments, the bronchodilator is albuterol, levalbuterol, salmeterol, formoterol, or any combination thereof. In some embodiments, the corticosteroid is racemic epinephrine, fluticasasone, budesonide, or any combination thereof. In some embodiments, the mast cell stabilizer or anti-IgE antibody is mometasone furoate, nedocromil, or any combination thereof. In some embodiments, the leukotriene receptor antagonist is cromolyn sodium, omalizumab, or any combination thereof. In some embodiments, the antihistamine is zafirlukast, montelukast, zileuton, or any combination thereof. In some embodiments, the respiratory stimulant is loratadine, fexofenadine, cetirizine, epinephrine, or any combination thereof. In some embodiments, the pulmonary surfactant is doxapram, theophylline, progesterone, caffeine, or any combination thereof. In some embodiments, the oxygen antimicrobial is colfosceril palmitate, beractant, calfactant, poractant alpha, or any combination thereof. In some embodiments, the antiviral is pentamidine, or tobramycin, or any combination thereof. In some embodiments, the expectorant is ribavirin, zanamivir, guaifenesin, varenicline, or any combination thereof. In some embodiments, the respiratory agent is almitrine, amiphenazole, AZD-5423, bemegride, BIMU8, budesonide/formoterol, BW373U86, CX-546, dimefline, doxapam, etamivan, GAL-021, leptacline, mepixanox, nikethamide, pentylenetetrazol, zacopride,

### Macrophage and Monocyte Activators

In some embodiments, the activator is a small molecule drug, an endotoxin, a cytokine, a chemokine, an interleukin, a pattern recognition receptor (PRR) ligand, a toll-like receptor (TLR) ligand, an adhesion molecule, or any combinations thereof. In some embodiments, the small molecule drug is phorbol myristate acetate. In some embodiments, the cytokine is IL-4, IL-13, interferon gamma (IFNγ), and/or tumor-necrosis factor (TNF). In some embodiments, the endotoxin is lipopolysaccharide (LPS) or endotoxin delta. In some embodiments, the adhesion molecule is an integrin, an immunoglobulin, or a selectin.

In some embodiments, the activator is a toll-like receptor (TLR) ligand, or a molecule that activates downstream TLR signaling. In some embodiments, the TLR ligand is a ligand that binds to TLR-1, TLR-2, TLR-3, TLR-4, TLR-5, TLR-6, TLR-7, TLR-8, TLR-9, TLR-10, TLR-11, TLR-12, or TLR-13. In some embodiments, the TLR ligand is a ligand that binds to TLR-3 or TLR-4. In some embodiments, the ligand of TLR-3 or TLR-4 is a pathogen-associated molecular pattern (PAMP). In some embodiments, the ligand that binds to TLR-3 is a double-stranded RNA. In some embodiments, the ligand that binds to TLR-4 is a lipopolysaccharide (LPS).

### Immune Cells & Antibodies

In some embodiments, the additional therapeutic agent is an additional immune cell. In some embodiments, the additional therapeutic agent is an antibody that binds to an unwanted pathogen (e.g., bacteria). In some embodiments, the additional immune cell is a T-cell, for example a helper T-cell (Tₕ cell) or a cytotoxic T-cell. In some embodiments, the T-cell is exposed to an antigen of an unwanted pathogen before administration to the individual. In some embodiments, the T-cell targets a specific antigen of an unwanted pathogen (e.g., bacteria). In some embodiments, the T-cell is does not target a specific antigen of an unwanted pathogen. In some embodiments, the additional immune cell is a dendritic cell. In some embodiments, the dendritic cell is exposed to an antigen of the unwanted pathogen before administration to the individual. In some embodiments, the dendritic cell targets a specific antigen of an unwanted pathogen (e.g., bacteria). In some embodiments, the additional immune cell is a B cell. In some embodiments, the B cell is exposed to an antigen of an unwanted pathogen before administration to the individual. In some embodiments, the B cell targets a specific antigen of an unwanted pathogen (e.g., bacteria). In some embodiments, the B cell expresses a B-cell receptor that binds to an antigen of the unwanted pathogen. In other embodiments, the additional therapeutic agent is a monocyte when the innate immune cell being administered to the individual is a macrophage. In some embodiments, the monocyte differentiates into a macrophage in vivo.

### Pharmaceutical Compositions

Disclosed herein, in certain embodiments, are pharmaceutical compositions comprising: (a) an isolated and purified innate immune cell; and (b) a pharmaceutically-acceptable excipient. Disclosed herein, in certain embodiments, are pharmaceutical compositions comprising: (a) an isolated and purified macrophage; and (b) a pharmaceutically-acceptable excipient. Disclosed herein, in certain embodiments, are pharmaceutical compositions comprising: (a) an isolated and purified monocyte; and (b) a pharmaceutically-acceptable excipient.

In some embodiments, the innate immune cell is isolated and purified by any of the methods disclosed herein. In some embodiments, the macrophage is isolated and purified by any of the methods disclosed herein. In some embodiments, the monocyte is isolated and purified by any of the methods disclosed herein. In some embodiments, a pharmaceutical composition includes one population of innate immune cells, or more than one, such as two, three, four, five, six or more populations of innate immune cells. In some embodiments, a pharmaceutical composition comprises a population of isolated and purified macrophages and a population of isolated and purified monocytes. In some embodiments, a pharmaceutical composition comprises a population of isolated and purified macrophages, a population of isolated and purified monocytes, and additional populations of isolated and purified innate immune cells.

In some embodiments, the components of the pharmaceutical compositions described herein are administered either alone or in combination with pharmaceutically acceptable carriers, excipients, or diluents, in a pharmaceutical composition. Pharmaceutical compositions are formulated in a conventional manner using one or more pharmaceutically acceptable inactive ingredients that facilitate processing of the active compounds into preparations that are used pharmaceutically. Pharmaceutically-acceptable excipients included in the pharmaceutical compositions will have different purposes depending, for example, on the subpopulation of innate immune cells used and the mode of administration. Non-limiting examples of generally used pharmaceutically-acceptable excipients include, without limitation: saline, buffered saline, dextrose, water-for-injection, glycerol, ethanol, dextran (e.g., low molecular dextran such as Dextran 40), PlasmaLyte, human serum albumin (HSA), and combinations thereof, stabilizing agents, solubilizing agents and surfactants, buffers and preservatives (such as dimethylsulfoxide (DMSO)), tonicity agents, bulking agents, and lubricating agents. The formulations comprising populations of innate immune cells are prepared and cultured in the absence of any non-human components, such as animal serum.

In some embodiments, the pharmaceutical compositions further comprise a compound that activates the innate immune cell. In some embodiments, the pharmaceutical compositions further comprise a compound that activates the macrophage. In some embodiments, the pharmaceutical compositions further comprise a compound that activates the monocyte. In some embodiments, the compound that activates the innate immune cell is selected from: IL-4, IL-13, phorbol myristate acetate, lipopolysaccharide (LPS), IFNγ, tumor-necrosis factor (TNF), or any combinations thereof. In some embodiments, the compound that activates the macrophage is selected from: IL-4, IL-13, phorbol myristate acetate, lipopolysaccharide (LPS), IFNγ, tumor-necrosis factor (TNF), or any combinations thereof. In some embodiments, the compound that activates the monocyte is selected from: IL-4, IL-13, phorbol myristate acetate, lipopolysaccharide (LPS), IFNγ, tumor-necrosis factor (TNF), or any combinations thereof.

In some embodiments, the pharmaceutical compositions further comprise a cryoprotectant or a cryopreservative. In some embodiments, the cryoprotectant or the cryopreservative is selected from dimethylsulfoxide (DMSO), formamide, propylene glycol, ethylene glycol, glycerol, trehalose, 2-methyl-2,4-pentanediol, methanol, butanediol, or any combination thereof.

Pharmaceutical compositions comprising: (a) an isolated and purified innate immune cell; and (b) a pharmaceutically-acceptable excipient are administered to a subject using modes and techniques known to the skilled artisan. Exemplary modes include, but are not limited to, intraperitoneal (I.P.) injection. Other modes include, without limitation, intravenous, intratumoral, intradermal, subcutaneous (S.C., s.q., sub-Q, Hypo), intramuscular (i.m.), intra-arterial, intramedullary, intracardiac, intra-articular (joint), intrasynovial (joint fluid area), intracranial, intraspinal, intrathecal (spinal fluids), intraduodenal, intramedullary, intraosseous, intrathecal, intravascular, intravitreal, and epidural. In some embodiments, any known device useful for parenteral (e.g., intraperitoneal) injection and/or infusion of the formulations is used to effect such administration.

In some embodiments, pharmaceutical compositions are formulated for parenteral administration by injection, e.g., by bolus injection or continuous infusion. Formulations for injection are presented in unit dosage form, e.g., in ampoules or in multi-dose containers, with an added preservative. In some embodiments, the compositions take such forms as suspensions, solutions, or emulsions in oily or aqueous vehicles, and contain formulatory agents such as suspending, stabilizing and/or dispersing agents. In some embodiments, the compositions are presented in unit-dose or multi-dose containers, for example sealed ampoules and vials, and are stored in powder form or in a freeze-dried (lyophilized) condition requiring only the addition of the sterile liquid carrier, for example, saline or sterile pyrogen-free water, immediately prior to use. In some embodiments, extemporaneous injection solutions and suspensions are prepared from sterile powders, granules and tablets of the kind previously described.

In some embodiments, pharmaceutical compositions for parenteral administration include aqueous and non-aqueous (oily) sterile injection solutions of the active compounds which contain antioxidants, buffers, bacteriostats, and solutes which render the formulation isotonic with the blood of the intended recipient; and aqueous and non-aqueous sterile suspensions which include suspending agents and thickening agents. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. In some embodiments, the suspension also contains suitable stabilizers or agents which increase the solubility of the compounds to allow for the preparation of highly concentrated solutions.

It should be understood that in addition to the ingredients particularly mentioned above, the compounds and compositions described herein include other agents conventional in the art having regard to the type of formulation in question.

### Methods of Dosing and Treatment Regimens

In certain embodiments, the compositions comprising the innate immune cells and/or the combination therapies described herein are administered for prophylactic and/or therapeutic treatments of diseases. In some embodiments, the activated macrophage compositions described herein are administered for prophylactic and/or therapeutic treatments of diseases. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions comprising the innate immune cells described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to an individual, who previously experienced at least one symptom of the disease being treated and is currently in remission, a pharmaceutical composition comprising an immune cell described herein, in order to prevent a return of the symptoms of the disease or condition.

In certain embodiments, an innate immune cell and an additional therapeutic agent described herein are administered at a dose lower than the dose at which either the innate immune cell or the additional therapeutic agent are normally administered as monotherapy agents. In certain embodiments, an innate immune cell and an additional therapeutic agent described herein are administered at a dose lower than the dose at which either the innate immune cell or the additional therapeutic agent are normally administered to demonstrate efficacy. In certain embodiments, an innate immune cell is administered at a dose lower than the dose at which it is normally administered as a monotherapy agent, when administered in combination with an additional therapeutic agent described herein. In certain embodiments, an innate immune cell is administered at a dose lower than the dose at which it is normally administered to demonstrate efficacy, when administered in combination with an additional therapeutic agent described herein. In certain embodiments, an additional therapeutic agent is administered at a dose lower than the dose at which it is normally administered as a monotherapy agent, when administered in combination with an innate immune cell. In certain embodiments, an additional therapeutic agent is administered at a dose lower than the dose at which it is normally administered to demonstrate efficacy, when administered in combination with an innate immune cell.

In certain embodiments, wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of the pharmaceutical composition being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the individual in need of treatment, but nevertheless is determined according to the particular circumstances surrounding the case, including, e.g., the specific agent being administered, the route of administration, the condition being treated, and the individual being treated.

In some embodiments, the pharmaceutical compositions comprising an innate immune cell are administered at a dosage in the range of about 10³ to about 10¹⁰ innate immune cells per kg of body weight innate immune cells per kg of body weight, including all integer values within those ranges. In some embodiments, the pharmaceutical compositions comprising a macrophage are administered at a dosage in the range of about 10³ to about 10¹⁰ macrophages per kg of body weight, preferably about 10⁵ to about 10⁶ macrophages per kg of body weight, including all integer values within those ranges. In some embodiments, the pharmaceutical compositions comprising a monocyte is administered at a dosage in the range of about 10³ to about 10¹⁰ monocytes per kg of body weight, preferably about 10⁵ to about 10⁶ monocytes per kg of body weight, including all integer values within those ranges. In one embodiment, the desired dose is conveniently presented in a single dose or in divided doses administered simultaneously or at appropriate intervals, for example as two, three, four or more sub-doses per day. In some embodiments, the desired dose is administered as a single dose or in divided doses within about 72 hours of each other. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual, the severity of the disease or condition being treated, and the judgment of the practitioner.

In some embodiments, the pharmaceutical compositions comprising an innate immune cell (e.g., an activated macrophage) are administered intraperitoneally to the individual. In some embodiments, the pharmaceutical compositions comprising an innate immune cell (e.g., an activated macrophage) are administered directly into the abdominal cavity. In some embodiments, the innate immune cells are administered intraperitoneally to the individual. In some embodiments, the activated macrophages are administered intraperitoneally to the individual. In some embodiments, the activated macrophages are administered through a drain placed in an abscess (e.g., an abdominal abscess) of an individual. In some embodiments, the activated macrophages are administered to the individual via a catheter (e.g., an intraperitoneal catheter).

In some embodiments, the pharmaceutical compositions comprising an innate immune cell (e.g., an activated macrophage) are administered at a dosage of about 10×10⁶ cells per kilogram (kg). In some embodiments, the pharmaceutical compositions comprising an innate immune cell (e.g., an activated macrophage) are administered at a dosage of about 12.5×10⁶ cells/kg. In some embodiments, the pharmaceutical compositions comprising an innate immune cell (e.g., an activated macrophage) are administered at a dosage of about 1,000 cells/kg to about 10,000,000,000 cells/kg. In some embodiments, the pharmaceutical compositions comprising an innate immune cell (e.g., an activated macrophage) are administered at a dosage of at least about 1,000 cells/kg. In some embodiments, the pharmaceutical compositions comprising an innate immune cell (e.g., an activated macrophage) are administered at a dosage of at most about 10,000,000,000 cells/kg. In some embodiments, the pharmaceutical compositions comprising an innate immune cell (e.g., an activated macrophage) are administered at a dosage of about 1,000 cells/kg to about 10,000 cells/kg, about 1,000 cells/kg to about 100,000 cells/kg, about 1,000 cells/kg to about 1,000,000 cells/kg, about 1,000 cells/kg to about 10,000,000 cells/kg, about 1,000 cells/kg to about 100,000,000 cells/kg, about 1,000 cells/kg to about 1,000,000,000 cells/kg, about 1,000 cells/kg to about 10,000,000,000 cells/kg, about 10,000 cells/kg to about 100,000 cells/kg, about 10,000 cells/kg to about 1,000,000 cells/kg, about 10,000 cells/kg to about 10,000,000 cells/kg, about 10,000 cells/kg to about 100,000,000 cells/kg, about 10,000 cells/kg to about 1,000,000,000 cells/kg, about 10,000 cells/kg to about 10,000,000,000 cells/kg, about 100,000 cells/kg to about 1,000,000 cells/kg, about 100,000 cells/kg to about 10,000,000 cells/kg, about 100,000 cells/kg to about 100,000,000 cells/kg, about 100,000 cells/kg to about 1,000,000,000 cells/kg, about 100,000 cells/kg to about 10,000,000,000 cells/kg, about 1,000,000 cells/kg to about 10,000,000 cells/kg, about 1,000,000 cells/kg to about 100,000,000 cells/kg, about 1,000,000 cells/kg to about 1,000,000,000 cells/kg, about 1,000,000 cells/kg to about 10,000,000,000 cells/kg, about 10,000,000 cells/kg to about 100,000,000 cells/kg, about 10,000,000 cells/kg to about 1,000,000,000 cells/kg, about 10,000,000 cells/kg to about 10,000,000,000 cells/kg, about 100,000,000 cells/kg to about 1,000,000,000 cells/kg, about 100,000,000 cells/kg to about 10,000,000,000 cells/kg, or about 1,000,000,000 cells/kg to about 10,000,000,000 cells/kg. In some embodiments, the pharmaceutical compositions comprising an innate immune cell (e.g., an activated macrophage) are administered at a dosage of about 1,000 cells/kg, about 10,000 cells/kg, about 100,000 cells/kg, about 1,000,000 cells/kg, about 10,000,000 cells/kg, about 100,000,000 cells/kg, about 1,000,000,000 cells/kg, or about 10,000,000,000 cells/kg.

In any of the aforementioned aspects are further embodiments in which the effective amount of the pharmaceutical compound described herein is: (a) systemically administered to the subject; and/or (and/or (c) intravenously administered to the subject; and/or (d) administered by injection to the subject; and/or (0 administered non-systemically or locally to the subject.

In any of the aforementioned aspects are further embodiments comprising single administrations of the effective amount of the pharmaceutical composition, including further embodiments in which (i) the pharmaceutical composition is administered once a day; or (ii) the pharmaceutical composition is administered to the individual multiple times over the span of one day.

In any of the aforementioned aspects are further embodiments comprising multiple administrations of the effective amount of the pharmaceutical composition, including further embodiments in which (i) the pharmaceutical composition is administered continuously or intermittently: as in a single dose; (ii) the time between multiple administrations is every 6 hours; (iii) the compound is administered to the individual every 8 hours; (iv) the compound is administered to the individual every 12 hours; (v) the compound is administered to the individual every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one pharmaceutical composition described herein, in combination with one or more other therapeutic agents.

In one embodiment, the therapeutic effectiveness of one of the pharmaceutical compositions described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the pharmaceutical compositions described herein with another agent (which also includes a therapeutic regimen) that also has therapeutic benefit.

In one specific embodiment, a pharmaceutical composition described herein, is co-administered with a second therapeutic agent, wherein the pharmaceutical composition described herein, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient may be additive of the two therapeutic agents or the patient may experience a synergistic benefit.

In certain embodiments, different dosages of the pharmaceutical composition disclosed herein are utilized in formulating pharmaceutical composition and/or in treatment regimens when the compounds disclosed herein are administered in combination with one or more additional agent, such as an additional drug, an adjuvant, or the like. Dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a pharmaceutical composition described herein, is initiated prior to, during, or after treatment with a second agent described herein, and continues until any time during treatment with the second agent or after termination of treatment with the second agent. It also includes treatments in which a pharmaceutical composition described herein, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the individual suffers; the age, weight, sex, diet, and medical condition of the individual). Thus, in some instances, the dosage regimen actually employed varies and, in some embodiments, deviates from the dosage regimens set forth herein.

For combination therapies described herein, dosages of the co-administered pharmaceutical compositions vary depending on the type of co-drug employed, on the specific drug employed, on the disease or condition being treated and so forth. In additional embodiments, when co-administered with one or more other therapeutic agents, the pharmaceutical composition provided herein is administered either simultaneously with the one or more other therapeutic agents, or sequentially.

In combination therapies, the multiple therapeutic agents (one of which is one of the pharmaceutical compositions described herein) are administered in any order or even simultaneously. If administration is simultaneous, the multiple therapeutic agents are, by way of example only, provided in a single, unified form, or in multiple forms (e.g., as a single pill or as two separate pills).

The pharmaceutical compositions described herein, or a pharmaceutically acceptable salt thereof, as well as combination therapies, are administered before, during or after the occurrence of a disease or condition, and the timing of administering the pharmaceutical composition containing a compound varies. Thus, in one embodiment, the pharmaceutical compositions described herein are used as a prophylactic and are administered continuously to individuals with a propensity to develop conditions or diseases in order to prevent the occurrence of the disease or condition. In another embodiment, the pharmaceutical compositions are administered to an individual during or as soon as possible after the onset of the symptoms. In specific embodiments, a pharmaceutical composition described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each individual. For example, in specific embodiments, a compound described herein or a formulation containing the pharmaceutical composition is administered for at least 2 weeks, about 1 month to about 5 years.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1—Generation of Allogenic Macrophages

A peripheral blood sample is obtained from the donor. The peripheral blood sample is subjected to gradient centrifugation to generate a buffy coat fraction. The buffy coat fraction is subjected to gradient centrifugation in the presence of Ficoll to generate a peripheral blood mononuclear cell (PBMC) fraction. The PBMC fraction is suspended in PBS-EDTA and centrifuged to generate an isolated PBMC pellet. The isolated PBMC pellet is suspended in RPMI 1640 medium to generate a solution of isolated PBMCs.

The solution of isolated PBMCs is subjected to gradient centrifugation in the presence of Percoll solution. The monocyte fraction is isolated, suspended in PBS-EDTA and centrifuged to generate an isolated monocyte pellet. The monocyte pellet is suspended in RPMI 1640 medium to generate a solution of isolated monocytes.

The isolated monocytes are contacted with granulocyte-macrophage (M-CSF) to generate differentiated macrophages.

The differentiated macrophages are contacted with IFNγ and tumor-necrosis factor (TNF) to generate activated macrophages.

The activated macrophages are frozen and stored for future use.

### Example 2—Allogenic Treatment of a Bacterial Infection

An individual presents with a fever. The physician diagnoses the individual as having a bacterial infection. The physician unfreezes and administers an allogenic supply of activated macrophages by intraperitoneal (IP) delivery to the individual.

### Example 3—Autologous Treatment of a Viral Infection

An individual presents with a fever. The physician diagnoses the individual as having a viral infection.

The physician obtains a peripheral blood sample from the individual. The peripheral blood sample is subjected to gradient centrifugation to generate a buffy coat fraction. The buffy coat fraction is subjected to gradient centrifugation in the presence of Ficoll to generate a peripheral blood mononuclear cell (PBMC) fraction. The PBMC fraction is suspended in PBS-EDTA and centrifuged to generate an isolated PBMC pellet. The isolated PBMC pellet is suspended in X-VIVO to generate a solution of isolated PBMCs.

A plurality of monocytes is isolated from the solution of isolated PBMCs. The solution of isolated PBMCs is subjected to positive selection using magnetic microbeads coated with anti-CD14 antibody (CD14 MicroBeads) in order to enrich the monocyte population. In other words, the solution of isolated PBMCs is magnetically labeled with the CD14 MicroBeads and loaded into a column, which is placed in the magnetic field of a separator. The magnetically labeled cells are retained within the column and the unlabeled cells run through and are depleted. In this manner, the magnetically retained CD14+ cells (i.e. the monocytes) are eluted as the positively selected cell fraction.

The monocyte fraction is isolated, suspended in PBS-EDTA and centrifuged to generate an isolated monocyte pellet. The monocyte pellet is suspended in X-VIVO to generate a solution of isolated monocytes.

The isolated monocytes are contacted with granulocyte-macrophage (GM-CSF) to generate differentiated macrophages.

The differentiated macrophages are contacted with phorbol myristate acetate, lipopolysaccharide (LPS), tumor-necrosis factor (TNF), IFNγ, or any combinations thereof to generate activated macrophages.

The activated macrophages are administered to the individual.

### Example 4—Generation of Allogenic Monocytes

A peripheral blood sample is obtained from the donor. The peripheral blood sample is subjected to gradient centrifugation to generate a buffy coat fraction. The buffy coat fraction is subjected to gradient centrifugation in the presence of Ficoll to generate a peripheral blood mononuclear cell (PBMC) fraction. The PBMC fraction is suspended in PBS-EDTA and centrifuged to generate an isolated PBMC pellet. The isolated PBMC pellet is suspended in RPMI 1640 medium to generate a solution of isolated PBMCs.

The solution of isolated PBMCs is subjected to gradient centrifugation in the presence of Percoll solution. The monocyte fraction is isolated, suspended in PBS-EDTA and centrifuged to generate an isolated monocyte pellet. The monocyte pellet is suspended in RPMI 1640 medium to generate a solution of isolated monocytes.

The isolated monocytes are frozen and stored for future use.

### Example 5—Treatment of a Bacterial Infection with Allogenic Monocytes

An individual presents with a fever. The physician diagnoses the individual as having a bacterial infection. The physician unfreezes and administers an allogenic supply of activated monocytes intraperitoneally to the individual.

### Example 6—Treatment of a Viral Infection with Autologous Monocytes

An individual presents with a fever. The physician diagnoses the individual as having a viral infection.

The physician obtains a peripheral blood sample from the individual. The peripheral blood sample is subjected to gradient centrifugation to generate a buffy coat fraction. The buffy coat fraction is subjected to gradient centrifugation in the presence of Ficoll to generate a peripheral blood mononuclear cell (PBMC) fraction. The PBMC fraction is suspended in PBS-EDTA and centrifuged to generate an isolated PBMC pellet. The isolated PBMC pellet is suspended in X-VIVO to generate a solution of isolated PBMCs.

A plurality of monocytes is isolated from the solution of isolated PBMCs. The solution of isolated PBMCs is suspended in phosphate buffered saline (PBS) containing 10% AB serum and is incubated for 10 minutes at 4° C. in order to block the nonspecific binding of monoclonal antibodies (Mab) to surface Fc receptors. The PBMCs are then centrifuged to form a pellet and the pellet is resuspended in a solution containing a fluorescently-labeled monocyte-specific Mab (e.g. Alexa Fluor® 488 anti-CD14 antibody). The monocytes are sorted and isolated using a flow cytometer with sorting capabilities.

The monocyte fraction is isolated, suspended in PBS-EDTA and centrifuged to generate an isolated monocyte pellet. The monocyte pellet is suspended in X-VIVO to generate a solution of isolated monocytes.

The isolated monocytes are grown in cell culture with X-VIVO to generate a plurality of monocytes.

The monocytes are administered to the individual.

### Example 7—IFNγ-Stimulated Mouse Macrophages Showed an Increased Killing of Multiple Bacterial Species, Including Multi-Drug Resistant Bacterial Species

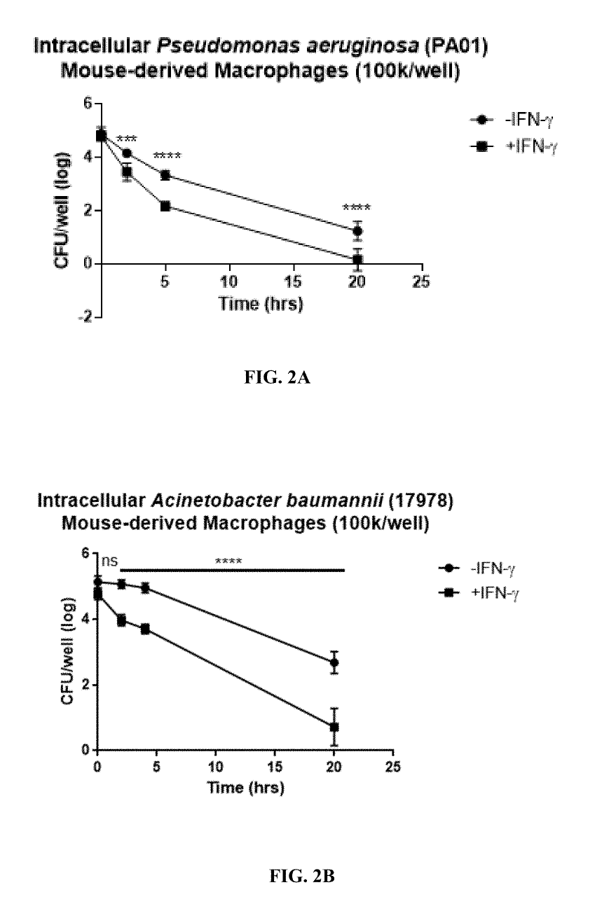

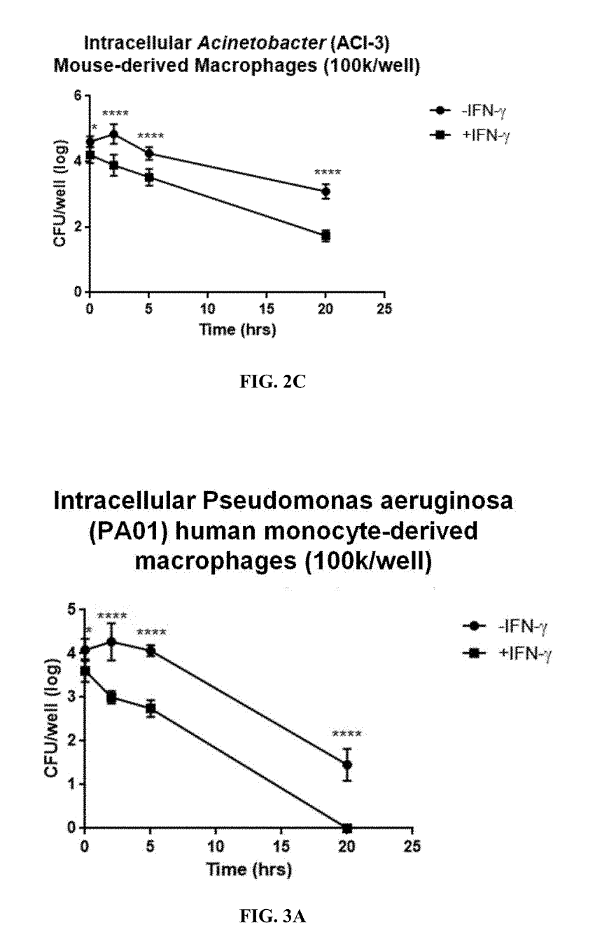

Bone marrow was obtained from the femurs of C57BL6 mice by flushing with PBS into a petri dish. The resulting cell slurry was passed through a cell strainer to remove clumps then subjected to centrifugation to pellet the cells. The pellet was resuspended in red blood cell lysis buffer followed by several rounds of centrifugation and PBS-wash to remove contaminating red blood cells. The isolated bone marrow cell pellet was suspended in RPMI 1640 medium supplemented with 10% fetal bovine serum and 20 ng/mL M-CSF and transferred to a tissue culture dish. Cultures were fed with fresh media at day 4 and were mature at day 7. In the case of activated macrophages, cells were stimulated overnight on day 7 with interferon gamma (IFNγ) prior to use. To determine anti-bacterial function, fully differentiated macrophages+/−IFNγ stimulation were first incubated with pathogens for 1 h at 37° C. At 1 h post infection, gentamicin was added to kill any extracellular bacteria and incubation was continued for 1 h at 37° C. At this time, the cells were washed twice with PBS and fresh media containing gentamicin was added. Samples were taken at indicated times and lysed to release surviving intracellular bacteria. These were enumerated by limiting dilution on agar plates and the number of colony forming units (CFU; measure of live bacteria) was determined following incubation of the agar plates at 37° C. for 18-24 h. The results of this experiment are shown in FIGS. 2A-C. Mouse bone marrow-derived macrophages stimulated with interferon gamma (IFNγ) (squares) exhibited an enhanced ability to kill virulent bacterial strains, as evidenced by a decrease in intracellular bacterial burden (CFU=colony forming units) (FIGS. 2A-C). The enhanced killing was seen with the clinically relevant species Pseudomonas aeruginosa (FIG. 2A), Acinetobacter baumannii. (FIG. 2B), and a multidrug resistant clinical isolate of Acinetobacter baumannii (ACI-3) (FIG. 2C). Data shown in FIGS. 2A-C is an average of 6 technical replicates from each of 4 biological replicates.

### Example 8—IFNγ-Stimulated Human Monocyte-Derived Macrophages Showed Increased Killing of Multiple Bacterial Species

A peripheral blood sample was obtained from the donor. The peripheral blood sample was subjected to gradient centrifugation in the presence of Ficoll and generated a peripheral blood mononuclear cell (PBMC) fraction. The PBMC fraction was suspended in PBS-EDTA and centrifuged to generate an isolated PBMC pellet. The pellet was resuspended in red blood cell lysis buffer followed by several rounds of centrifugation and PBS-EDTA wash to remove contaminating red blood cells. The isolated PBMC pellet was suspended in Hanks Balanced Salt Solution (HBSS)—EDTA and the PBMC were subjected to positive selection using beads coated with anti-CD14 antibody to enrich the monocyte population. The monocyte fraction was isolated, suspended in PBS-EDTA and centrifuged to generate an isolated monocyte pellet. The monocyte pellet was suspended in RPMI 1640 medium to generate a solution of isolated monocytes. The isolated monocytes were suspended in RPMI 1640 medium supplemented with 10% fetal bovine serum and 125 ng/mL M-CSF and transferred to a tissue culture dish. Cultures were fed with fresh media at day 4 and are mature at day 7. In the case of activated macrophages, cells were stimulated overnight on day 7 with interferon gamma (IFNγ) prior to use. To determine anti-bacterial function, fully differentiated macrophages+/−IFNγ stimulation were first incubated with pathogens for 1 h at 37° C. At 1 h post infection, gentamicin was added to kill any extracellular bacteria and incubation was continued for 1 h at 37° C. At this time, the cells were washed twice with PBS and fresh media containing gentamicin was added. Samples were taken at indicated times and lysed to release surviving intracellular bacteria. These were enumerated by limiting dilution on agar plates and the number of colony forming units (CFU; measure of live bacteria) was determined following incubation of the agar plates at 37° C. for 18-24 h.

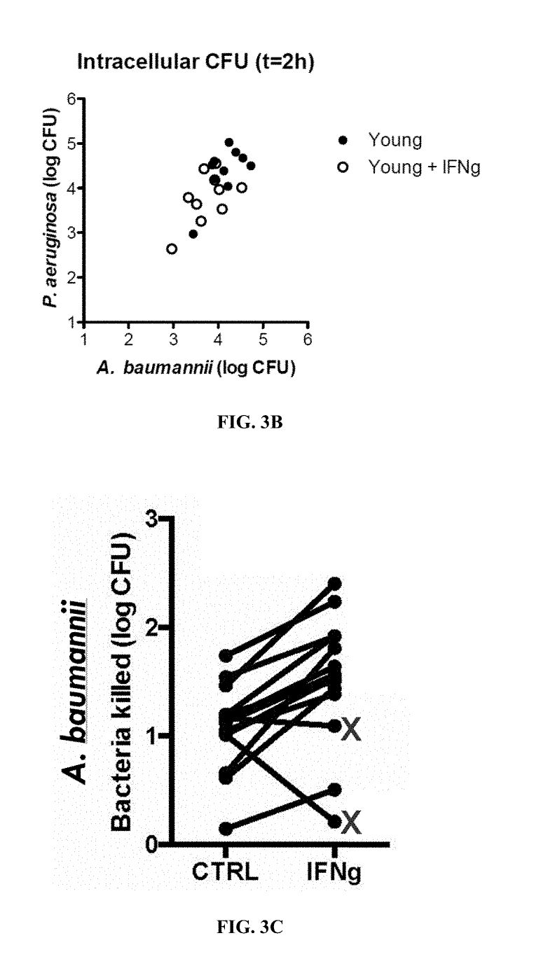

The results of this experiment are shown in FIG. 3A-C. Human monocyte-derived macrophages stimulated with IFNγ increased the killing of multiple bacterial species. FIG. 3A shows the total bacterial burden over time (t=20 hrs) before and after exposure to human monocyte-derived macrophages stimulated with interferon gamma (IFNγ) (squares). As evidenced by a decrease in intracellular bacterial burden (CFU=colony forming units), human monocyte-derived macrophages stimulated with interferon gamma (IFNγ) had an enhanced ability to kill Pseudomonas aeruginosa. FIG. 3B shows the number of bacteria killed by monocyte-derived macrophages over the course of 2 hrs. The monocyte-derived macrophages obtained from different donors (n=14) and stimulated with IFNγ showed enhanced killing across multiple clinically relevant species, with a correlation between activities against different bacterial species (p=0.002). FIG. 3C compares the number of bacteria killed by human monocyte-derived macrophages stimulated with IFNγ and a control (non-stimulated human monocyte-derived macrophages). IFNγ stimulated human monocyte-derived macrophages to kill A. baumannii in a majority of young adult donors (8 of 10 donors).

### Example 9—Infusion of Mouse Monocyte-Derived Macrophages Decreased Organ Bacterial Load In Vivo

Two groups of 10 C57/black female mice were infected IP with 10⁶ A. baumannii. In the treatment group each mouse was treated with 10⁷ activated macrophages delivered intraperitoneally (i.p.) at t=1 h post-infection. Macrophages were bone marrow-derived macrophages activated with 12 ng/ml IFN-γ for 24 h prior to IP injection in mice. Clinical signs were monitored every 8 hours for a 22 hour period. Moribund mice (clinical score being equal to or greater than 4) were sacrificed and organs were harvested for CFU counts (heart, liver, spleen). No significant differences in clinical signs or survival were noted between the two groups of animals. However, one of the treated animals scored a 2 at 22 h post infection. This animal was sacrificed at 22 h post-injection.

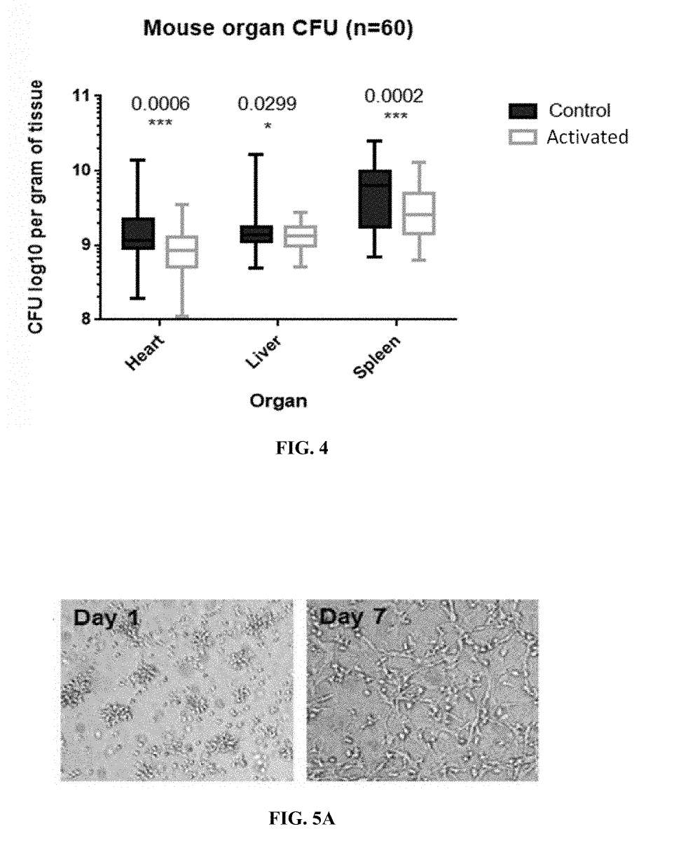

The results of this study are shown in FIG. 4. FIG. 4 shows the infusion of mouse monocyte-derived macrophages decreases organ bacterial load in vivo. Mice injected intraperitoneally with Acinetobacter baumanni were subsequently injected with either Control (unstimulated; n=10 animals) or Activated (IFNγ stimulated; n=9 animals) mouse-derived macrophages. Animals were sacrificed and bacterial load (CFU=colony forming units) was measured. Animals treated with stimulated macrophages showed significantly lower bacterial burden in multiple organs. Data shown represents technical triplicates from each organ.

### Example 10—Treatment of a Complicated Intra-Abdominal Infection (cIAI) with Cryopreserved Allogenic Mononuclear Phagocytes

An individual presents with a fever, tachycardia, abdominal pain, nausea, and vomiting. The physician diagnoses the individual as having a cIAI caused by acute appendicitis. The physician performs an appendectomy, administers the standard of care anti-microbial therapy, and administers an allogenic supply of activated mononuclear phagocytes by intraperitoneal (IP) infusion to the individual. The physician continues to administer a series of infusions of activated, allogenic mononuclear phagocytes over the course of 1 week through a catheter drain placed during surgery.

The mononuclear phagocytes are generated by isolating a specific precursor white blood cell (CD34⁺ HSC or CD14⁺ monocytes) from human blood (whole blood, mobilized blood, or cord blood or apheresis product) and then differentiating and stimulating them to enhance their anti-microbial function across a spectrum of infectious agents. Differentiated and stimulated mononuclear phagocytes are frozen, stored, and then thawed prior to infusion.

### Example 11—Treatment of a Complicated Intra-Abdominal Infection (cIAI) with Fresh Allogenic Mononuclear Phagocytes

An individual presents with a fever, tachycardia, abdominal pain, nausea, abdominal rigidity, and vomiting. The physician diagnoses the individual as having a cIAI caused by cholecystitis. The physician performs a cholecystectomy, administers the standard of care anti-microbial therapy, and administers an allogenic supply of activated mononuclear phagocytes by IP infusion to the individual. The physician continues to administer a series of infusions of activated, allogenic mononuclear phagocytes over the course of 2 weeks through a catheter drain placed during surgery.

The mononuclear phagocytes are generated by isolating a specific precursor white blood cell (CD34⁺ HSC or CD14⁺ monocytes) from human blood (whole blood, mobilized blood, or cord blood) and then differentiating and stimulating them to enhance their anti-microbial function across a spectrum of infectious agents. Differentiated and stimulated mononuclear phagocytes are used fresh prior to infusion.

### Example 12—Validation of Cell Surface Markers and Secreted Cytokine Profile Correlated with In Vitro Bacterial Cell Killing Activity

In another example, the efficiency of monocyte to macrophage differentiation following ex vivo culture was assessed. Purified CD14+ monocytes were cultured in macrophage colony-stimulating factor (M-CSF) in order to drive macrophage differentiation. After 7 days of ex vivo culture, cells transitioned from small rounded monocytes (labeled as “Day 1” in FIG. 5A) to larger, heterogeneously shaped macrophage-like cells, some of which show a spindle-shaped morphology (labeled as “Day 7”), as shown in FIG. 5A. To examine the efficiency of monocyte to macrophage conversion during ex vivo culture, expression of macrophage-associated marker CD206 following differentiation was examined by flow cytometry over a large cohort of donors and was found to be highly expressed, as shown in the flow cytometry histogram of FIG. 5B. The expression of CD206 was calculated to have a mean of 89.6% positive CD206 expression (with a standard deviation, denoted as “SD,” of 11.71) from a total of 12 donors, as shown in the scatter plot of FIG. 5B. These macrophages also expressed markers associated with IFNγ-mediated stimulation such as CD38. The expression of CD38 was examined via flow cytometry. FIG. 5C shows a flow cytometry histogram showing an elevated expression of CD38 in the differentiated macrophages (right peak) compared to controls (left peak). The mean fluorescent intensity (MFI) was calculated and compared to controls (i.e., fold change MFI) and is shown in the plot of FIG. 5C. The expression of CD38 had a mean fold change of MFI of 3.492 with a standard deviation, denoted as “SD,” of 1.35. Additionally, to further examine the efficiency of the monocyte to macrophage conversion during ex vivo culture, a more unbiased approach, LEGENDScreen (BioLegend) technology, was used to assess the expression of 361 cell surface markers. The differentiated cells were highly enriched for macrophage associated markers (e.g., CD206, CD163, CD63, CD14) and macrophage activation markers (e.g., CD38 and CD86) and displayed low expression of cell surface markers associated with a wide variety of other hematopoietic and non-hematopoietic cell types, as shown in FIG. 7A. Analysis of a panel of cytokines and chemokines secreted into the culture medium showed that in vitro anti-bacterial activity correlated with expression and secretion of known inflammatory macrophage markers such as tumor necrosis factor alpha (TNFα), chemokine (C—C motif) ligand 5 (CCL5)/regulated on activation, normal T cell expressed and secreted (RANTES), and interferon gamma-induced protein 10 (IP-10), as shown in FIG. 7B. In addition, the differentiated macrophages did not express non-myeloid markers (<1%); a finding critical for ensuring the cells do not induce graft-versus-host disease. FIG. 7B shows the concentration of cytokines in picograms per milliliter (pg/mL) found in the culture medium.

### Example 13—In Vitro Microbiological Assay for Breadth of Species Assessment

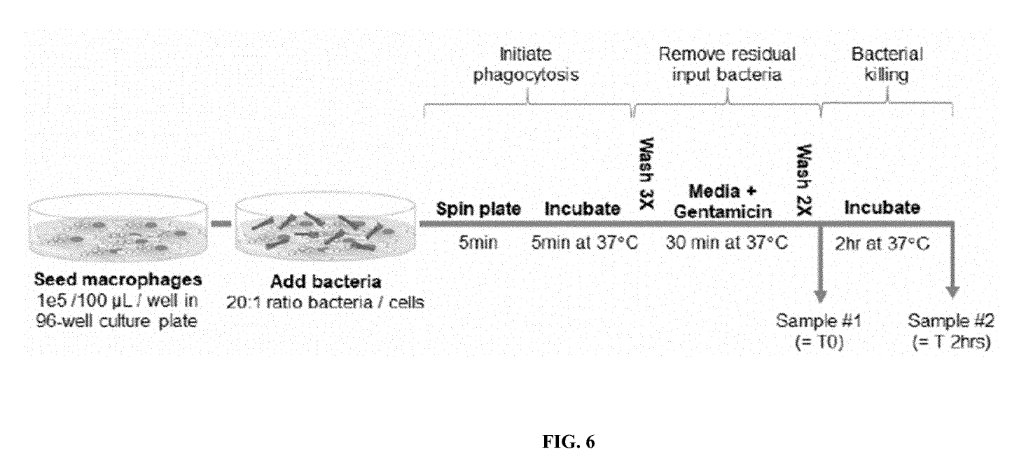

In another example, a modified version of the gentamicin protection assay is carried out to assess activity across a panel of relevant susceptible and drug-resistant strains (FIG. 6). Briefly, 1×10⁵ (i.e., 10,000) human monocyte-derived macrophages stimulated with interferon gamma (IFNγ) grown in xenobiotic-free culture conditions are plated into the wells of 96-well tissue culture plates. After adherence, bacteria in suspension are added to the culture wells, and the plates are centrifuged to maximize bacterial/macrophage interactions prior to brief culture (about 5 minutes) at 37° C. to initiate the phagocytosis process. The cultures are then washed and briefly pulsed with the cell-impermeable antibiotic gentamicin to kill the remaining live extracellular bacteria. This process is necessary because trace amounts of live extracellular bacteria grow aggressively in mammalian culture media, preventing the ability to measure the activity of the macrophages. After this pulse, the cultures are washed and resuspended in mammalian culture medium and a sample is lysed and subjected to colony forming unit (CFU) analysis (denoted as “=TO” in FIG. 6). The cells are then cultured for an additional two hours at 37° C. to enable bacterial cell killing to occur followed by lysis and CFU analysis. The level of live bacteria (i.e., CFU) at the 2 hour time point (denoted as “=T 2 hrs” in FIG. 6) is compared to Time 0 to assess the fraction of bacterial killing for that particular bacterial species.

### Example 14—Functional Assays of Stimulated Macrophages

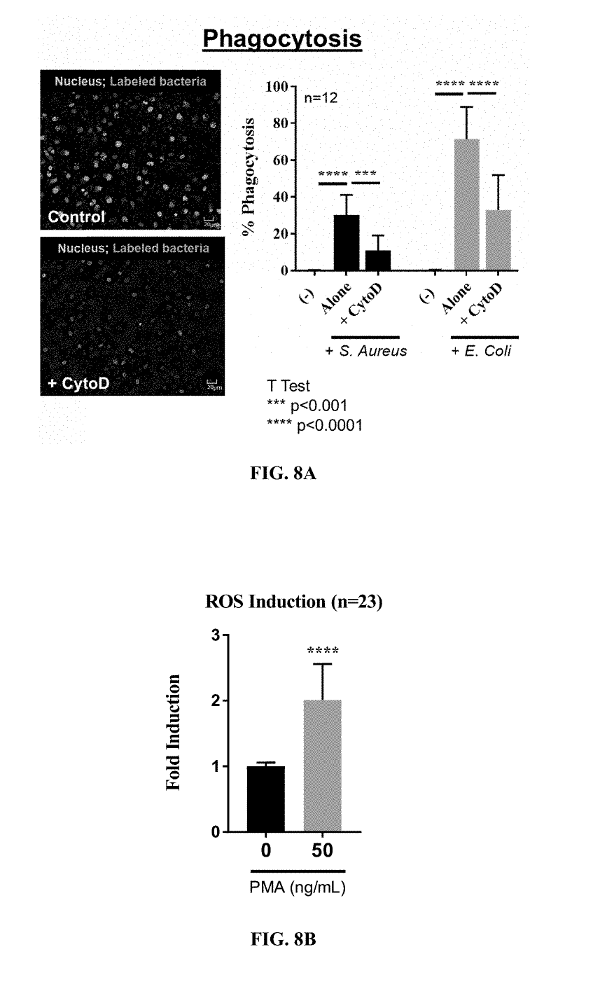

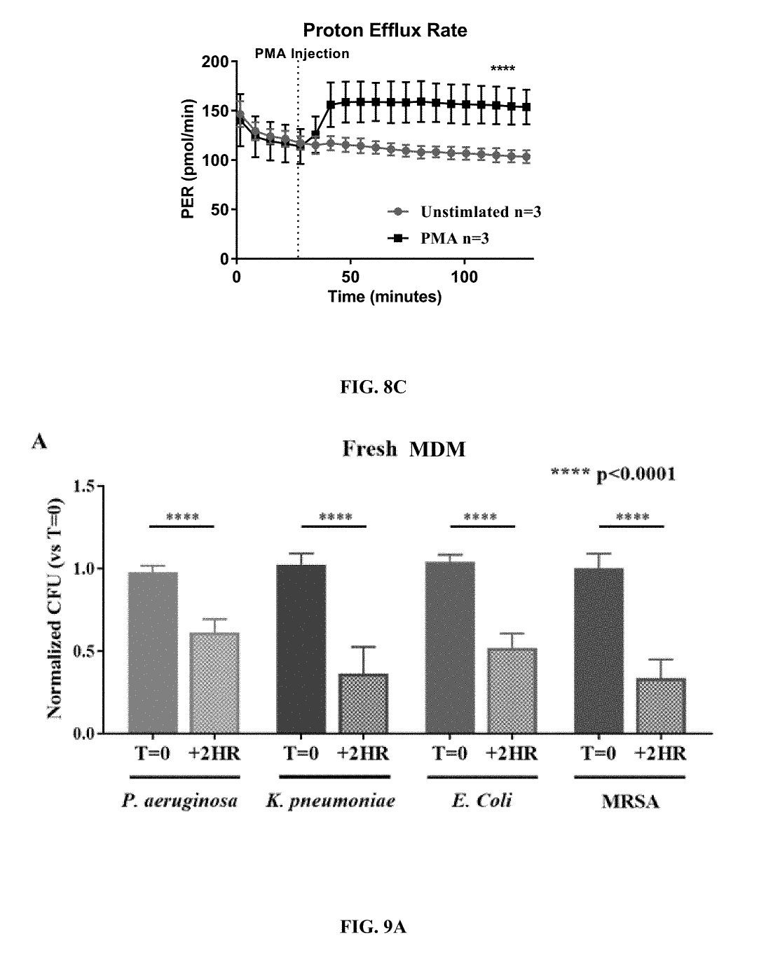

In another example, a number of functional assays were performed to evaluate different functions of the differentiated macrophages such as phagocytosis, reactive oxygen species (ROS) production, and proton efflux rate production. FIG. 8A shows an evaluation of the phagocytic function of the differentiated macrophages. In this study, heat-killed bacteria are labeled with a pH sensitive dye that only fluoresces when the bacteria have been phagocytosed and delivered to the lysosome, the site of bacterial degradation within the macrophage. Phagocytosis was found to be inhibited by an inhibitor of actin polymerization called cytochalasin D (CytoD) as shown by the lack of labeled bacteria in the fluorescence microscopy image of macrophages with labeled nuclei in the presence of CytoD (FIG. 8A, bottom image). In contrast, a fluorescence microscopy image of the control (i.e., macrophages with labeled nuclei without cytochalasin D) show the presence of phagocytosed labeled bacteria (FIG. 8A, top image). Furthermore, the percent of phagocytosis was measured using a flow cytometry readout. FIG. 8A shows the quantification of the percentage of phagocytosis of labeled heat-killed S. Aureus and E. coli by stimulated macrophages, and further in the presence and absence of cytochalasin D. The percent of phagocytosis of macrophages exposed to cytochalasin D (denoted as “+CytoD” in FIG. 8A) was lower than the percent of phagocytosis in the absence of cytochalasin D (denoted as “Alone” in FIG. 8A). This trend was observed for both bacterial species (i.e., S. Aureus and E. coli), and the total number of donor macrophages tested per bacterial species was 12 (denoted as “n=12” in FIG. 8A).

The production of reactive oxygen species (ROS) by stimulated macrophages was measured. Macrophages were stimulated with 50 nanograms/milliliter (ng/mL) of phorbol 12-myristate 13-acetate (PMA). As seen in FIG. 8B, the fold induction of stimulated macrophages versus non-stimulated macrophages was about a 1-fold increase. The number of donor macrophages tested was 23 (denoted as “n=23” in FIG. 8B). This result demonstrated the expected production of reactive oxygen species.

Furthermore, the proton efflux rate (PER) was measured in stimulated and unstimulated macrophages. The PER of the macrophages begins to differ once a subset of the macrophages is activated by contacting them with PMA (denoted as “PMA Injection” in FIG. 8C). As shown in FIG. 8C, macrophages stimulated with PMA generated a PER of about 150 picomole per minute (pmol/min) after 100 minutes. In contrast, unstimulated macrophages only generated a PER of about 100 pmol/min. The number of samples measured was 3 (denoted as “n=3” in FIG. 8C). The number of donor macrophages tested was 3 (denoted as n=3 in FIG. 8C).

### Example 15—Validation of Human Macrophage Activity Against Bacterial Pathogens Relevant in cIAI

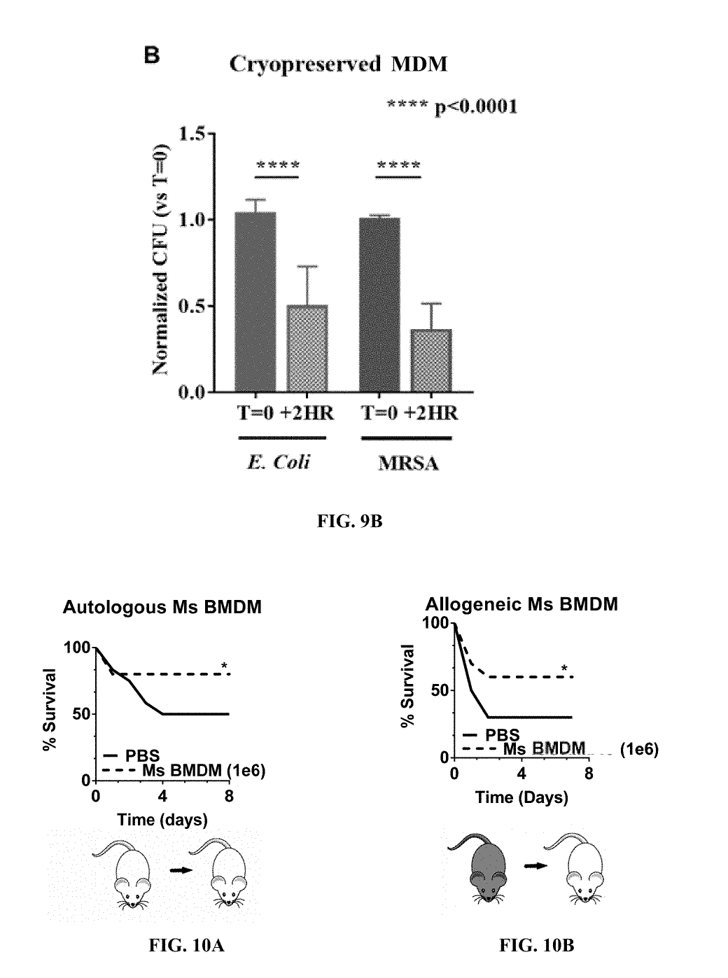

In another example, in vitro studies were conducted to ensure broad-spectrum activity of human monocyte-derived macrophages across relevant drug-resistant bacterial species (e.g., Gram-positive and Gram-negative). In addition, these studies were conducted using culture media devoid of the xenobiotic additives common in laboratory settings. Both fresh CD14+ monocyte-derived macrophages (MDM) (FIG. 9A) and cryopreserved CD14+ monocyte-derived macrophages (MDM) (FIG. 9B) exhibited broad-spectrum activity against multi-drug resistant (MDR) Gram-positive (GP; MRSA) and Gram-negative (GN; carbapenem-resistant E. coli K. pneumoniae, and P. aeruginosa) bacterial isolates, measured by the decline in colony forming units (CFU) at 2-hours post infection. The pathogens shown in FIGS. 9A and 9B are typically associated with complicated intra-abdominal infections (cIAI). This activity was consistently seen across a panel of human donors (n=5), further supporting the utility of these cells in controlling a broad-spectrum of bacterial species as well as providing evidence of feasibility for generating macrophages in a therapeutically-friendly culture medium.

### Example 16—Validation of Mouse and Human Macrophage Efficiency in a Peritonitis Infection Model

In another example, the mouse and human macrophage efficiency in an in vivo MRSA rodent peritonitis model relevant to complicated intra-abdominal infections (cIAI) was assessed. Direct bacterial cell killing is only one aspect of macrophage control of bacterial infection; thus, the activity of CD14+ monocyte-derived macrophages (MDM) in an in vivo peritonitis model relevant to cIAI was examined.

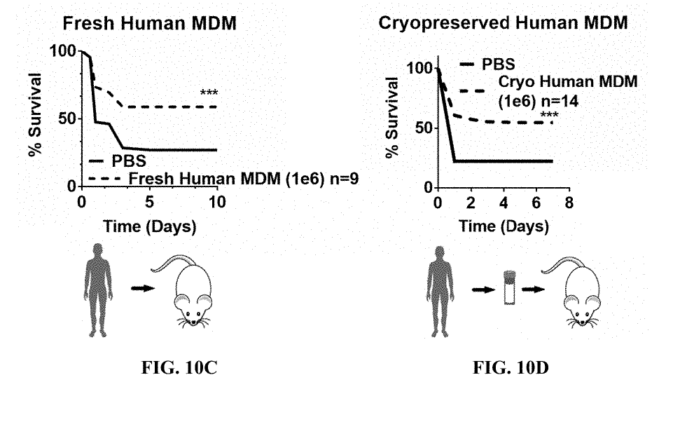

We first tested the ability of mouse bone-marrow derived macrophages (BMDM) stimulated with IFNγ to protect against lethality due to peritoneal MRSA infection in CD1 outbred mice. Autologous (CD1-derived) (FIG. 10A) mouse bone marrow-derived macrophages (BMDM) and allogeneic (C57/BL6-derived) mouse bone marrow-derived macrophages (BMDM) (FIG. 10B) significantly protected CD1 outbred mice from bacterial-induced lethality (p<0.05) when delivered IP, as shown in FIGS. 10A and 10B, respectively. We then tested the ability of xenobiotic-free human monocyte-derived macrophages (MDM) stimulated with IFNγ to confer protection in the immunocompetent mouse peritonitis model. Similar to the findings with mouse macrophages, fresh human MDM (FIG. 10C) and cryopreserved human MDM (FIG. 10D) were able to protect against lethality due to peritoneal MRSA infection in CD1 outbred mice when delivered IP compared to controls, as shown in FIGS. 10C and 10D (p<0.001).

In all four studies shown in FIGS. 10A, 10B, 10C, and 10D, the negative control was phosphate buffer saline (PBS), and mice were administered about 1×10⁶ cells (“1e6,” as shown in FIGS. 10A-D). Mice treated with stimulated mouse macrophages, as shown in FIGS. 10A and 10B, had a percent survival of about 65% after 10 days. On the other hand, mice treated with PBS (i.e., negative control) had a percent survival of about 20% after 10 days. Mice treated with stimulated human macrophages, as shown in FIGS. 10C and 10D, had a percent survival of about 70% after 10 days. Furthermore, mice treated with PBS (i.e., negative control) had a percent survival of about 30% after 10 days. Together, these in vivo findings further support the utility of stimulated macrophages in treating cIAI.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-58. (canceled)

59. A method of treating a complicated intra-abdominal infection (cIAI) in an individual in need thereof, comprising: administering to the individual a composition comprising an activated, allogenic macrophage and an excipient.

60. The method of claim 59, wherein the complicated intra-abdominal infection (cIAI) infection is a bacterial infection.

61. The method of claim 60, wherein the bacterial infection comprises gram negative bacteria.

62. The method of claim 60, wherein the bacterial infection comprises gram positive bacteria.

63. The method of claim 60, wherein the bacterial infection comprises multi-drug resistant bacteria, extensively drug resistant bacteria, or pan-drug resistant bacteria.

64. The method of claim 60, wherein the bacterial infection comprises bacteria that are resistant to an antibacterial drug selected from the group consisting of: penicillin, ampicillin, carbapenem, fluoroquinolone, cephalosporin, tetracycline, erythromycin, methicillin, gentamicin, vancomycin, imipenem, ceftazidime, levofloxacin, linezolid, daptomycin, ceftaroline, clindamycin, fluconazole, and ciprofloxacin.

65. The method of claim 60, wherein the bacterial infection comprises bacteria selected from the group consisting of: Lactobacillus, Klebsiella pneumoniae, Klebsiella pneumoniae resistant to third generation cephalosporin, Klebsiella oxytoca, Klebsiella oxytoca resistant to third generation cephalosporin, Clostridium, Clostridium difficile, Acinetobacter baumannii, Escherichia coli, Escherichia coli resistant to third generation cephalosporin, Pseudomonas, Pseudomonas aeruginosa, Staphylococcus aureus, Streptococcus spp., Streptococcus pyogenes, Enterobacteriaceae, Enterococcus faecium, Enterococcus faecalis, Helicobacter pylori, Streptococcus pneumoniae, Streptococcus agalactiae, Serratia, Stenotrophomonas maltophilia, Corynebacterium, Peptostreptococcus, Peptococcus, Staphylococcus epidermidis, Enterococcus, Enterobacter, Proteus, gram-positive anaerobic cocci (GPAC), Bacteroides fragilis, Proteus mirabilis, Bacteroides, Bacteroides resistant to metronidazole, and Morganella morganii.

66. The method of claim 60, wherein the bacterial infection comprises methicillin-resistant staphylococcus aureus (MRSA) bacteria.

67. The method of claim 60, wherein the bacterial infection comprises Escherichia coli bacteria.

68. The method of claim 60, wherein the bacterial infection comprises Klebsiella pneumoniae bacteria.

69. The method of claim 59, wherein the macrophage is activated ex vivo by contact with an activator.

70. The method of claim 69, wherein the activator is selected from: a small molecule drug, an endotoxin, a cytokine, a chemokine, an interleukin, a pattern recognition receptor (PRR) ligand, a toll-like receptor (TLR) ligand, an adhesion molecule, or any combinations thereof.

71. The method of claim 70, wherein the endotoxin is lipopolysaccharide (LPS) or delta endotoxin.

72. The method of claim 70, wherein the cytokine is IL-4, IL-13, or tumor-necrosis factor (TNF).

73. The method of claim 69, wherein the activator is interferon gamma (IFNγ).

74. The method of claim 59, wherein the macrophage is cryopreserved.

75. The method of claim 59, wherein the complicated intra-abdominal infection (cIAI) is associated with peritonitis.

76. The method of claim 59, wherein the complicated intra-abdominal infection (cIAI) is associated with appendicitis, intra-abdominal sepsis, an intra-abdominal abscess, an abdominal surgery, a gastrointestinal perforation, cholecystitis, diverticulitis, a postoperative abdominal infection, a colorectal surgery, or any combinations thereof.

77. The method of claim 59, wherein the macrophage is administered at a therapeutically effective dose of at least about 1 million macrophages per kilogram of body weight of the individual.

78. The method of claim 59, wherein the excipient is a cryoprotectant.
